(19) United States
(12) Patent Application Publication
(10) Pub. No.: US 2021/0230281 A1
(43) Pub. Date: Jul. 29, 2021

(54) DOSING OF A BISPECIFIC ANTIBODY THAT BIND CD123 AND CD3

(71) Applicant: NOVARTIS AG, Basel (CH)

(72) Inventors: Michael Wayne SAVILLE, San Diego, CA (US); Paul FOSTER, Sante Fe, CA (US)

(21) Appl. No.: 17/051,056

(22) PCT Filed: Apr. 26, 2019
(86) PCT No.: PCT/US2019/029319

§ 371 Date: Oct. 27, 2020

Related U.S. Application Data
(60) Provisional application No. 62/664,030, filed on Apr. 27, 2018.
(60) Provisional application No. 62/713,433, filed on Aug. 1, 2018.
(60) Provisional application No. 62/774,795, filed on Dec. 3, 2018.
(60) Provisional application No. 62/774,796, filed on Dec. 3, 2018.

(51) Int. Cl. C07K 16/28; A61K 39/395; A61K 45/06; A61K 31/138; A61K 31/167; A61P 35/02; A61K 31/573; A61P 35/00

(52) U.S. Cl. CPC C07K 16/2866; A61K 39/3955; A61K 45/06; A61K 31/138; A61K 31/167; A61K 2039/505; A61P 35/02; A61K 31/573; A61P 35/00; C07K 2317/31; C07K 16/2809

(57) ABSTRACT

The methods described here are directed to treating human subjects with bispecific anti-CD 123×anti-CD3 antibodies.

### CROSS-REFERENCE TO RELATED APPLICATIONS

This application claims the priority benefit of U.S. Prov. Appl. Nos. 62/774,796 filed Dec. 3, 2018; 62/774,795 filed Dec. 3, 2018; 62/713,433 filed Aug. 1, 2018; and 62/664,030 filed Apr. 27, 2018; the contents of which are incorporated herein by reference in their entireties.

### SEQUENCE LISTING

The instant application contains a Sequence Listing which has been submitted electronically in ASCII format and is hereby incorporated by reference in its entirety. The ASCII copy, created on Apr. 23, 2019, is named 067461-5220-WO_ST25.txt and is 45,423 bytes in size.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification controls.

### BACKGROUND OF THE INVENTION

Antibody-based therapies have been used successfully to treat a variety of diseases, including cancer and autoimmune/inflammatory disorders. Improvements to this class of antibodies are still needed, particularly with respect to enhancing their clinical efficacy. One avenue being explored is the engineering of additional and novel antigen binding sites into an antibody such that a single immunoglobulin molecule co-engages two different antigens.

CD3 activation of T-cells occurs only when its associated T-cell receptor (TCR) engages antigen-loaded MHC on antigen presenting cells in a highly avid cell-to-cell synapse (Kuhns et al., 2006, Immunity 24:133-139). Indeed, nonspecific bivalent cross-linking of CD3 using an anti-CD3 antibody elicits a cytokine storm and toxicity (Perruche et al., 2009, J Immunol 183[2]:953-61; Chatenoud & Bluestone, 2007, Nature Reviews Immunology 7:622-632; expressly incorporated by reference). Thus, for practical clinical use, the preferred mode of CD3 co-engagement for redirected killing of target cells is monovalent binding that results in activation only upon engagement with the co-engaged target.

CD123, also known as interleukin-3 receptor alpha (IL-3Rα), is expressed on dendritic cells, monocytes, eosinophils and basophils. CD123 is also constitutively expressed by committed hematopoietic stem/progenitor cells, by most of the myeloid lineage (CD13+, CD14+, CD33+, CD15_{low}), and by some CD19+ cells. It is absent from CD3+ cells.

There is a need for improved bispecific anti-CD-123×anti-CD3 antibodies and the use of such antibodies for use in therapy.

### BRIEF SUMMARY OF THE INVENTION

In one aspect, disclosed herein is a method for treating a CD123-expressing cancer in a human subject in need of treatment thereof, comprising administering to the human subject a bispecific anti-CD123×anti-CD3 antibody in at least a first and a second phase, where during the first phase, the bispecific anti-CD123×anti-CD3 antibody is administered to the human subject in an amount of between about 700 ng/kg and about 1,900 ng/kg, once a week, for one or two weeks, and where during the second phase, the bispecific anti-CD123×anti-CD3 antibody is administered to the human subject in an amount of between about 2,000 ng/kg and about 5,000 ng/kg, once a week, for at least one week.

In an embodiment, during the first and/or second phase, the bispecific anti-CD123×anti-CD3 antibody is administered over about two hours.

In an embodiment, the second phase has a duration of one or two weeks.

In an embodiment, the second phase is maintained until remission.

In an embodiment, further comprising administering a maintenance dose.

In an embodiment, the maintenance dose comprises the same amount of the bispecific anti-CD123×anti-CD3 antibody administered in the second phase.

In an embodiment, the maintenance dose is administered once every two weeks for at least one dose.

In an embodiment, the maintenance dose is administered once every three or four weeks or once a month for at least one dose.

In an embodiment, further comprising a third phase where the bispecific anti-CD123×anti-CD3 antibody is administered to the human subject in an amount of between about 3,000 ng/kg and about 11,000 ng/kg, once a week for at least one week.

In an embodiment, during the third phase, the bispecific anti-CD123×anti-CD3 antibody is administered over about two hours.

In an embodiment, the third phase has a duration of one or two weeks.

In an embodiment, the third phase is maintained until remission.

In an embodiment, further comprising administering a maintenance dose.

In an embodiment, the maintenance dose comprises the same amount of the bispecific anti-CD123×anti-CD3 antibody administered in the third phase.

In an embodiment, the maintenance dose is administered once every two weeks for at least one dose.

In an embodiment, the maintenance dose is administered once every three or four weeks or once a month for at least one dose.

In an embodiment, further comprising a fourth phase, where the bispecific anti-CD123×anti-CD3 antibody is administered to the human subject in an amount of between about 3,000 ng/kg and about 11,000 ng/kg, once a week for at least one week.

In an embodiment, during the fourth phase, the bispecific anti-CD123×anti-CD3 antibody is administered over about two hours.

In an embodiment, the fourth phase is maintained until remission.

In an embodiment, further comprising administering a maintenance dose.

In an embodiment, the maintenance dose comprises the same amount of the bispecific anti-CD123×anti-CD3 antibody administered in the fourth phase.

In an embodiment, the maintenance dose is administered once every two weeks for at least one dose.

In an embodiment, the maintenance dose is administered once every three or four weeks or once a month for at least one dose.

In an embodiment, during the first phase, the bispecific anti-CD123×anti-CD3 antibody is administered to the human subject in an amount of between about 1,150 ng/kg and about 1,450 ng/kg.

In an embodiment, during the first phase, the bispecific anti-CD123×anti-CD3 antibody is administered to the human subject in an amount of between about 700 ng/kg and about 800 ng/kg.

In an embodiment, the method consists essentially of a first phase and a second phase, where the first phase is one week, and where during the second phase, the bispecific anti-CD123×anti-CD3 antibody is administered to the human subject in an amount of between about 2,200 ng/kg and about 2,400 ng/kg, once a week, until remission.

In an embodiment, the method consists essentially of a first, second, and third phase, where the first phase is one week, where during the second phase, the bispecific anti-CD123×anti-CD3 antibody is administered to the human subject in an amount of between about 2,200 ng/kg and about 2,400 ng/kg, once a week, for two weeks, and where during the third phase, the bispecific anti-CD123×anti-CD3 antibody is administered to the human subject in an amount of between about 3,750 ng/kg and about 4,250 ng/kg, once a week, until remission.

In an embodiment, the method consists essentially of a first, second, third, and fourth phase, where the first phase is one week, where during the second phase, the bispecific anti-CD123×anti-CD3 antibody is administered to the human subject in an amount of between about 1,200 ng/kg and about 2,400 ng/kg, once a week, for one week, where during the third phase, the bispecific anti-CD123×anti-CD3 antibody is administered to the human subject in an amount of between about 3,750 ng/kg and about 4,250 ng/kg, once a week, for one week, and where during the fourth phase, the bispecific anti-CD123×anti-CD3 antibody is administered to the human subject in an amount of between about 6,500 ng/kg and about 7,500 ng/kg, once a week, until remission.

In an embodiment, the method consists essentially of a first, second, third, and fourth phase, where the first phase is one week, where during the second phase, the bispecific anti-CD123×anti-CD3 antibody is administered to the human subject in an amount of between about 3,750 ng/kg and about 4,250 ng/kg, once a week, for one week, where during the third phase, the bispecific anti-CD123×anti-CD3 antibody is administered to the human subject in an amount of between about 6,500 ng/kg and about 7,500 ng/kg, once a week, for one week, and where during the fourth phase, the bispecific anti-CD123×anti-CD3 antibody is administered to the human subject in an amount of between about 11,000 ng/kg and about 13,000 ng/kg, once a week, until remission.

In an embodiment, the bispecific anti-CD123×anti-CD3 antibody is administered intravenously.

In an embodiment, during the third and/or fourth phases, the bispecific anti-CD123×anti-CD3 antibody is administered over about two hours.

In another aspect, disclosed herein is a method for treating a CD123-expressing cancer in a human subject in need of treatment thereof, comprising administering to the human subject a bispecific anti-CD123×anti-CD3 antibody in at least a first phase and a second phase and a third phase, where during the first phase, the bispecific anti-CD123×anti-CD3 antibody is administered to the human subject in an amount of between about 300 ng/kg and about 1,100 ng/kg, three times a week, for one week, with the proviso that the first dose amount of the first phase is not greater than about 770 ng/kg, where during the second phase, the bispecific anti-CD123×anti-CD3 antibody is administered to the human subject in an amount of between about 300 ng/kg and about 1,100 ng/kg, three times a week, for one week, and where during the third phase, the bispecific anti-CD123×anti-CD3 antibody is administered to the human subject in an amount of between about 900 ng/kg and about 3,400 ng/kg, once a week for at least one week.

In an embodiment, during the first phase, the bispecific anti-CD123×anti-CD3 antibody is administered to the human subject in an amount of between about 400 ng/kg and about 450 ng/kg, three times a week, for one week, and where during the second phase, the bispecific anti-CD123×anti-CD3 antibody is administered to the human subject in an amount of between about 400 ng/kg and about 450 ng/kg, three times a week, for one week where during the third phase, the bispecific anti-CD123×anti-CD3 antibody is administered to the human subject in an amount of between about 1,150 ng/kg and about 1,450 ng/kg, once a week for at least one week.

In an embodiment, during the first phase, the bispecific anti-CD123×anti-CD3 antibody is administered to the human subject, three times a week, for one week, where the first dose amount in the first phase is about 750 ng/kg, and the subsequent two dose amounts in the first phase are between about 760 ng/kg and about 780 ng/kg and where during the second phase, the bispecific anti-CD123×anti-CD3 antibody is administered to the human subject in an amount of between about 760 ng/kg and about 780 ng/kg, three times a week, for one week, and where during the third phase, the bispecific anti-CD123×anti-CD3 antibody is administered to the human subject in an amount of between about 2,200 ng/kg and about 2,400 ng/kg, once a week for at least one week.

In an embodiment, during the first phase, the bispecific anti-CD123×anti-CD3 antibody is administered to the human subject, three times a week, for one week, where the first dose amount in the first phase is about 750 ng/kg, and the subsequent two dose amounts in the first phase are between about 1,150 ng/kg and about 1,450 ng/kg where during the second phase, the bispecific anti-CD123×anti-CD3 antibody is administered to the human subject in an amount of between about 1,150 ng/kg and about 1,450 ng/kg, three times a week, for one week, and where during the third phase, the bispecific anti-CD123×anti-CD3 antibody is administered to the human subject in an amount of between about 3,750 ng/kg and 4,250 ng/kg, once a week for at least one week.

In an embodiment, during the first and/or second and/or third phase, the bispecific anti-CD123×anti-CD3 antibody is administered over about two hours.

In an embodiment, the bispecific anti-CD123×anti-CD3 antibody is administered intravenously.

In an embodiment, the second phase is maintained until remission.

In an embodiment, further comprising administering a maintenance dose.

In an embodiment, the maintenance dose comprises the same amount of the bispecific anti-CD123×anti-CD3 antibody administered in the second phase.

In an embodiment, the maintenance dose is administered once every two weeks for at least one dose.

In an embodiment, the maintenance dose is administered once every three or four weeks or once a month for at least one dose.

In another aspect, disclosed herein is a method for treating a CD123-expressing cancer in a human subject in need of treatment thereof, comprising administering to the human subject a bispecific anti-CD123×anti-CD3 antibody in an amount of between about 900 ng/kg and about 3,400 ng/kg, once a week for at least one week.

In an embodiment, the bispecific anti-CD123×anti-CD3 antibody is administered in an amount of between about 1,150 ng/kg and 1,450 ng/kg.

In an embodiment, the bispecific anti-CD123×anti-CD3 antibody is administered in an amount of between about 2,200 ng/kg and 2,400 ng/kg.

In an embodiment, the CD123-expressing cancer is a hematologic cancer.

In an embodiment, the CD123-expressing cancer is a leukemia.

In an embodiment, the CD123-expressing cancer is selected from the group consisting of acute myeloid leukemia (AML), chronic myeloid leukemia (CML), acute lymphocytic leukemia (ALL), and hairy cell leukemia (HCL).

In an embodiment, the CD123-expressing cancer is acute myeloid leukemia (AML).

In an embodiment, the acute myeloid leukemia (AML) is blastic plasmacytoid dendritic cell neoplasm (BPDCN).

In an embodiment, the CD123-expressing cancer is acute lymphocytic leukemia, and the acute lymphocytic leukemia is B-cell acute lymphocytic leukemia (B-ALL).

In an embodiment, the remission is a reduction in the number of CD123-expressing cancer cells or reduction in the rate of growth of CD123-expressing cancer cells.

In an embodiment, the remission is an increase in T cell activation or an increase in IFN pathway upregulation.

In an embodiment, the remission is a partial remission of the CD123-expressing cancer.

In an embodiment, the bispecific anti-CD123×anti-CD3 antibody comprises a Heavy Chain 1 (HC1) (Fab-Fc) set forth in SEQ ID NO:1, a Heavy Chain 2 (HC2) (scFv-Fc) set forth in SEQ ID NO: 2 and a Light Chain set forth in SEQ ID NO: 3.

In an embodiment, the bispecific anti-CD123×anti-CD3 antibody consists of a Heavy Chain 1 (HC1) (Fab-Fc) set forth in SEQ ID NO:1, a Heavy Chain 2 (HC2) (scFv-Fc) set forth in SEQ ID NO: 2 and a Light Chain set forth in SEQ ID NO: 3.

In an embodiment, further comprising assessing the weight of the human subject prior to the administering of the first phase of the bispecific anti-CD123×anti-CD3 antibody.

In an embodiment, further comprising administering to the human subject one or more therapeutic agents prior to the administering of the first phase of the bispecific anti-CD123×anti-CD3 antibody.

In an embodiment, the one or more therapeutic agents ameliorates the side effects of the bispecific anti-CD123×anti-CD3 antibody administration.

In an embodiment, the one or more therapeutic agents is a steroid, an antihistamine, an anti-allergic agent, an antinausea agent (or anti-emetic), an analgesic agent, an antipyretic agent, a cytoprotective agent, a vasopressor agent, an anticonvulsant agent, an anti-inflammatory agent, or any combination thereof.

In an embodiment, the one or more therapeutic agents is a combination of a corticosteroid, diphenhydramine, and acetaminophen.

### BRIEF DESCRIPTION OF THE DRAWINGS

FIG. 1 depicts a useful bispecific antibody, the format of which is referred to as a “bottle opener”. XmAb14045 is in this bottle opener format. It should be noted that the scFv and Fab domains can be switched (e.g., anti-CD3 as a Fab and anti-CD123 as a scFv).

FIG. 2 depicts the sequences of the three polypeptide chains that make up XmAb14045, a bispecific anti-CD123×anti-CD3 antibody. The CDRs are underlined and the junction between domains is denoted by a slash (“/”). The charged scFv linker is double underlined; the linker may be substituted with other linkers, for example, linkers that are depicted in FIG. 7 of U.S. Pat. Appl. Pub. No. 2014/0288275 or other non-charged linkers such as SEQ ID NO:441 of U.S. Pat. Appl. Pub. No. 2014/0288275.

FIG. 3 depicts the different anti-CD123 Fab constructs that were engineered to increase affinity to human CD123 and to increase the stability of the 7G3 H1L1 construct (see, e.g., U.S. Pat. Appl. Pub. No. 2016/0229924, FIG. 136, SEQ ID NOs: 455 and 456). The changes to the amino acid sequences are shown.

FIG. 4 depicts the affinity and stability properties of optimized humanized variants of the parental 7G3 murine antibody (see, e.g., U.S. Pat. Appl. Pub. No. 2016/0229924, FIG. 136, NOs: 453 and 454).

FIG. 5A-5B depict additional anti-CD123 Fab sequences with the CDRs underlined.

FIG. 6 depicts additional anti-CD123×anti-CD3 sequences. The CDRs are underlined and the junction between domains is denoted by a slash (“/”). The charged scFv linker is double underlined; the linker may be substituted with other linkers, for example, linkers that are depicted in FIG. 7 of U.S. Pat. Appl. Pub. No. 2014/0288275 or other non-charged linkers such as SEQ ID NO:441 of U.S. Pat. Appl. Pub. No. 2014/0288275.

FIGS. 7A-7D depicts additional bispecific formats, as are generally described in FIG. 1 and the accompanying legend and supporting text of U.S. Pat. Appl. Pub. No. 2016/0229924.

FIG. 8 depicts RTCC with intact or T cell depleted PBMC against KG-1a target cells. Effector cells (400 k), intact or magnetically-depleted PBMC were incubated with carboxyfluorescein succinimidyl ester-labeled KG-1a target cells (10 k) for 24 hours and stained with annexin V for cell death.

FIG. 9 depicts CD123hiCD33hi depletion over a dose range of XmAb14045 in AML human subject PBMC. Five AML human subject PBMC samples were incubated with a dose range of XmAb14045 (0.12 to 90 ng/mL) for 6 days, and live cells were gated to count CD123hiCD33hi target cells. The lowest concentration (0.04 ng/mL) point is the no drug control for plotting on logarithmic scale. Each point is normalized to account for cell count variability.

FIG. 10 depicts Ki67 levels in T cells from AML human subject PBMC with XmAb14045. Five AML human subject PBMC samples were incubated with a dose range of XmAb14045 (0.12 to 90 ng/mL) for 6 days, and live cells were gated for CD4+ and CD8+ T cells to count Ki67+ cells. The lowest concentration (0.04 ng/mL) point is the no drug control, for plotting on a logarithmic scale.

FIG. 11 depicts number of AML blasts in human subject PBMCs treated with XmAb14045. PBMC from a single AML human subject was incubated with 9 or 90 ng/mL XmAb14045 for 24 or 48 hours and blast counts were plotted. Normal donor PBMCs were also used as a control.

FIG. 12 depicts leukemic blast cells in AML human subject PBMC. PBMCs from six AML human subjects were incubated with antibodies for 48 hours and blasts were counted and plotted. One donor (AML #1) did not have XENP13245 treatment and each line is a single donor.

FIG. 13 depicts KG-1a tumor cell apoptosis with AML PBMC. Carboxyfluorescein succinimidyl ester-labeled CD123+ KG-1a cells were added to the PBMC to examine target cell cytotoxicity stimulated by the AML effector T cells. Staining with the apoptosis marker annexin-V was used to detect KG-1a cell death after 48 hours of incubation.

FIG. 14 depicts effect of XmAb14045 on tumor burden over time in a mouse xenograft model of AML.

FIG. 15 depicts reduction of tumor burden after 3 once a week doses of XmAb14045.

FIG. 16 depicts effect of XmAb14045 on T cell number in a mouse xenograft model of AML. Peripheral blood CD45+CD8+ events by flow cytometry. Samples taken on Day 11 and 20 after XmAb14045 administration.

FIG. 17 depicts CRS severity by infusion (Cohorts 9A-2B) from a subset of tested human subjects.

FIG. 18 depicts peak serum IL-6 by infusion from a subset of tested human subjects.

FIG. 19 depicts percentage change in bone marrow blasts from pretreatment baseline from a subset of tested human subjects.

FIG. 20 depicts the time to treatment discontinuation from a subset of tested human subjects.

FIG. 21 depicts CR and CRi responder data from a subset of tested human subjects.

FIG. 22 depicts blast CD123 expression, for responders versus non-responders, from a subset of tested human subjects.

### DETAILED DESCRIPTION OF THE INVENTION

### I. Definitions

In order that the application may be more completely understood, several definitions are set forth below. The definitions also include all grammatical equivalents.

The term “about” in relation to a reference numerical value can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value. In some cases, the numerical disclosed throughout can be “about” that numerical value even without specifically mentioning the term “about.”

Embodiments herein with the term ‘comprise’, ‘comprises’ or ‘comprising’ can have this term replaced with ‘consists of’ or ‘consisting of’ or ‘consists essentially of’ or ‘consisting essentially of’.

The terms “CD3” or “cluster of differentiation 3” means a T-cell co-receptor that helps in activation of both cytotoxic T-cell (e.g., CD8+ naïve T cells) and T helper cells (e.g., CD4+ naïve T cells) and is composed of four distinct chains: one CD3γ chain (e.g., Genbank Accession Numbers NM_000073 and MP_000064 (human)), one CD3δ chain (e.g., Genbank Accession Numbers NM_000732, NM_001040651, NP_00732 and NP_001035741 (human)), and two CD3ε chains (e.g., Genbank Accession Numbers NM_000733 and NP_00724 (human)). The chains of CD3 are highly related cell-surface proteins of the immunoglobulin superfamily containing a single extracellular immunoglobulin domain. CD3 molecule associates with the T-cell receptor (TCR) and ζ-chain to form the T-cell receptor (TCR) complex, which functions in generating activation signals in T lymphocytes.

The terms “CD123”, “Cluster of Differentiation 123”, “CD123 antigen”, “interleukin-3 receptor alpha”, “IL3RA”, or “interleukin3 receptor subunit alpha” means an interleukin 3 specific subunit of a type I heterodimeric cytokine receptor (e.g., Genbank Accession Numbers NM_001267713, NM_002183, NP_001254642 and NP_002174 (human)). CD123 interacts with a signal transducing beta subunit to form interleukin-3 receptor, which helps in the transmission of interleukin 3. CD123 is found on pluripotent progenitor cells and induces tyrosine phosphorylation within the cell and promotes proliferation and differentiation within the hematopoietic cell lines. CD123 is expressed across acute myeloid leukemia (AML) subtypes, including leukemic stem cells.

By “bispecific” or “bispecific antibody” herein is meant any non-native or alternate antibody formats, including those described herein, that engage two different antigens (e.g., CD3×CD123 bispecific antibodies).

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g. the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233# designates a deletion a deletion of glutamic acid at position 233. Additionally, EDA233- or EDA233# designates a deletion of the sequence GluAspAla that begins at position 233.

By “variant protein” or “protein variant”, or “variant” as used herein is meant a protein that differs from that of a parent protein by virtue of at least one amino acid modification. Protein variant may refer to the protein itself, a composition comprising the protein, or the amino sequence that encodes it. Preferably, the protein variant has at least one amino acid modification compared to the parent protein, e.g. from about one to about seventy amino acid modifications, and preferably from about one to about five amino acid modifications compared to the parent. As described below, in some embodiments the parent polypeptide, for example an Fc parent polypeptide, is a human wild type sequence, such as the Fc region from IgG1, IgG2, IgG3 or IgG4, although human sequences with variants can also serve as “parent polypeptides”. The protein variant sequence herein will preferably possess at least about 80% identity with a parent protein sequence, and most preferably at least about 90% identity, more preferably at least about 95-98-99% identity. Variant protein can refer to the variant protein itself, compositions comprising the protein variant, or the DNA sequence that encodes it. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain. The Fc variants of the present invention are defined according to the amino acid modifications that compose them. Thus, for example, N434S or 434S is an Fc variant with the substitution serine at position 434 relative to the parent Fc polypeptide, where the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as M428L/N434S, and so on. For all positions discussed in the present invention that relate to antibodies, unless otherwise noted, amino acid position numbering is according to the EU index. The EU index or EU index as in Kabat or EU numbering scheme refers to the numbering of the EU antibody (Edelman et al., 1969, Proc Natl Acad Sci USA 63:78-85, hereby entirely incorporated by reference.) The modification can be an addition, deletion, or substitution. Substitutions can include naturally occurring amino acids and, in some cases, synthetic amino acids. Examples include U.S. Pat. No. 6,586,207; WO 98/48032; WO 03/073238; US2004-0214988A1; WO 05/35727A2; WO 05/74524A2; J. W. Chin et al., (2002), Journal of the American Chemical Society 124:9026-9027; J. W. Chin, & P. G. Schultz, (2002), ChemBioChem 11:1135-1137; J. W. Chin, et al., (2002), PICAS United States of America 99:11020-11024; and, L. Wang, & P. G. Schultz, (2002), Chem. 1-10, all entirely incorporated by reference.

As used herein, “protein” herein is meant at least two covalently attached amino acids, which includes proteins, polypeptides, oligopeptides and peptides. The peptidyl group may comprise naturally occurring amino acids and peptide bonds, or synthetic peptidomimetic structures, i.e. “analogs”, such as peptoids (see Simon et al., PNAS USA 89(20):9367 (1992), entirely incorporated by reference). The amino acids may either be naturally occurring or synthetic (e.g. not an amino acid that is coded for by DNA); as will be appreciated by those in the art. For example, homo-phenylalanine, citrulline, ornithine and norleucine are considered synthetic amino acids for the purposes of the invention, and both D- and L-(R or S) configured amino acids may be utilized. The variants of the present invention may comprise modifications that include the use of synthetic amino acids incorporated using, for example, the technologies developed by Schultz and colleagues, including but not limited to methods described by Cropp & Shultz, 2004, Trends Genet. 20(12):625-30, Anderson et al., 2004, Proc Natl Acad Sci USA 101 (2):7566-71, Zhang et al., 2003, 303(5656):371-3, and Chin et al., 2003, Science 301(5635):964-7, all entirely incorporated by reference. In addition, polypeptides may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “antigen binding domain” or “ABD” herein is meant a set of six Complementary Determining Regions (CDRs) that, when present as part of a polypeptide sequence, specifically binds a target antigen as discussed herein. Thus, a “checkpoint antigen binding domain” binds a target checkpoint antigen as outlined herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 for the heavy chain and vlCDR1, vlCDR2 and vlCDR3 for the light. The CDRs are present in the variable heavy and variable light domains, respectively, and together form an Fv region. Thus, in some cases, the six CDRs of the antigen binding domain are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh. In general, the C-terminus of the scFv domain is attached to the N-terminus of the hinge in the second monomer.

By “Fab” or “Fab region” as used herein is meant the polypeptide that comprises the VH, CH1, VL, and CL immunoglobulin domains, as, for example, on two different polypeptide chains (e.g. VH-CH1 on one chain and VL-CL on the other). Fab may refer to this region in isolation, or this region in the context of a bispecific antibody, or this region in the context of a full-length antibody, antibody fragment or Fab fusion protein. In the context of a Fab, the Fab can comprise an Fv region in addition to the CH1 and CL domains.

By “Fv” or “Fv fragment” or “Fv region” as used herein is meant a polypeptide that comprises the VL and VH domains of an ABD. Fv regions can be formatted as both Fabs (as discussed above, generally two different polypeptides that also include the constant regions as outlined above) and scFvs, where the vl and vh domains are combined (generally with a linker as discussed herein) to form an scFv.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the figures, the order of the vh and vl domain is indicated in the name, e.g. H.X_L.Y means N- to C-terminal is vh-linker-vl, and L.Y_H.X is vl-linker-vh.

By “amino acid” and “amino acid identity” as used herein is meant one of the 20 naturally occurring amino acids that are coded for by DNA and RNA.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor”, “FcγR” or “FcqammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants can be used to increase binding to the FcRn receptor, and in some cases, to increase serum half-life.

By “parent polypeptide” as used herein is meant a starting polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below.

By “Fc” or “Fc region” or “Fc domain” as used herein is meant the polypeptide comprising the CH2-CH3 domains of an IgG molecule, and in some cases, inclusive of the hinge. In EU numbering for human IgG1, the CH2-CH3 domain comprises amino acids 231 to 447, and the hinge is 216 to 230. Thus the definition of “Fc domain” includes both amino acids 231-447 (CH2-CH3) or 216-447 (hinge-CH2-CH3), or fragments thereof. An “Fc fragment” in this context may contain fewer amino acids from either or both of the N- and C-termini but still retains the ability to form a dimer with another Fc domain or Fc fragment as can be detected using standard methods, generally based on size (e.g. non-denaturing chromatography, size exclusion chromatography, etc.) Human IgG Fc domains are of particular use in the present invention, and can be the Fc domain from human IgG1, IgG2 or IgG4.

By “heavy chain constant region” herein is meant the CH1-hinge-CH2-CH3 portion of an antibody (or fragments thereof), excluding the variable heavy domain; in EU numbering of human IgG1 this is amino acids 118-447 By “heavy chain constant region fragment” herein is meant a heavy chain constant region that contains fewer amino acids from either or both of the N- and C-termini but still retains the ability to form a dimer with another heavy chain constant region.

By “position” as used herein is meant a location in the sequence of a protein. Positions may be numbered sequentially, or according to an established format, for example the EU index for antibody numbering.

By “target antigen” as used herein is meant the molecule that is bound specifically by the antigen binding domain comprising the variable regions of a given antibody. The two target antigens of the present invention are human CD3 and human CD123.

By “strandedness” in the context of the monomers of the heterodimeric antibodies of the invention herein is meant that, similar to the two strands of DNA that “match”, heterodimerization variants are incorporated into each monomer so as to preserve the ability to “match” to form heterodimers. For example, if some pI variants are engineered into monomer A (e.g. making the pI higher) then steric variants that are “charge pairs” that can be utilized as well do not interfere with the pI variants, e.g. the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer that incorporates one set of the pair will go, such that pI separation is maximized using the pI of the skews as well.

By “target cell” as used herein is meant a cell that expresses a target antigen.

By “host cell” in the context of producing a bispecific antibody according to the invention herein is meant a cell that contains the exogenous nucleic acids encoding the components of the bispecific antibody and is capable of expressing the bispecific antibody under suitable conditions. Suitable host cells are discussed herein.

By “variable region” or “variable domain” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, C D. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc) are used. In one embodiment, sequence identity is done using the BLAST algorithm, using default parameters.

By “wild type or WT” herein is meant an amino acid sequence or a nucleotide sequence that is found in nature, including allelic variations. A WT protein has an amino acid sequence or a nucleotide sequence that has not been intentionally modified.

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogenous host cells, and they can be isolated as well.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵M, at least about 10⁻⁶ M, at least about 10⁻⁷M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore, SPR or BLI assay.

As used herein, the term “target activity” refers to a biological activity capable of being modulated by a selective modulator. Certain exemplary target activities include, but are not limited to, binding affinity, signal transduction, enzymatic activity, tumor growth, and effects on particular biomarkers related to CD123 disorder pathology.

By “refractory” in the context of a cancer is intended the particular cancer is resistant to, or non-responsive to, therapy with a particular therapeutic agent. A cancer can be refractory to therapy with a particular therapeutic agent either from the onset of treatment with the particular therapeutic agent (i.e., non-responsive to initial exposure to the therapeutic agent), or as a result of developing resistance to the therapeutic agent, either over the course of a first treatment period with the therapeutic agent or during a subsequent treatment period with the therapeutic agent.

As used herein, the IC₅₀ refers to an amount, concentration or dosage of a particular test compound that achieves a 50% inhibition of a maximal response, such as inhibition of the biological activity of CD123, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

The term “remission” in relation to cancer means a decrease in or disappearance of signs (e.g., tumor size, biomarkers) and/or symptoms of cancer. In some cases, the remission can be partial or complete. For example, remission can lead to the reduction or amelioration or elimination of the progression, severity and/or effect associated with a CD123-expressing cancer (e.g., a hematological cancer) and/or an improvement in one or more symptoms associated with a CD123-expressing cancer. In some cases, remission can be associated with an increase in the immune system response of the human subject, or the amelioration of one or more symptoms of a CD123-expressing cancer, that result from the administration of an antibody described herein. In cases, remission can result in the amelioration of at least one measurable physical parameter of a cancer, such as tumor size, rate of tumor growth, number of tumor cells, tumor invasiveness, presence of metastasis, or extent of metastasis. In other cases, remission can lead to the inhibition of the progression of a CD123-expressing cancer, either physically by, e.g., stabilization of a discernible symptom, physiologically by, e.g., stabilization of a physical parameter, or both. In some cases, remission can be associated with one or more of the following: (1) a reduction in the number of CD123⁺ expressing cancer-associated cells, including CD123⁺ peripheral blood blasts and/or marrow blasts, such as for example a reduction to levels below the detection limits of a MRD (minimal residual disease) assay (i.e. flow cytometry assay, RT-qPCR assay, or next-gen sequencing based MRD assay); (2) an increase in CD123⁺ expressing cancer-associated cell death; (3) inhibition of CD123⁺ expressing cancer-associated cell survival; (5) inhibition (i.e., slowing to some extent, preferably halting) of CD123⁺ expressing cancer-associated proliferation; (6) an increased human subject survival rate; (7) improvement in peripheral blood cytopenias associated with the CD123-expressing cancer; and (8) any amount of relief (subjective and/or objective) from one or more symptoms of a CD123-expressing cancer.

Remission can be determined by standardized response criteria specific to that CD123-expressing cancer. Examples of such response criteria include the European LeukemiaNet response assessment categories for clinical trials. Examples for AML can be found in Dohner et al. Blood, 2017; 129(4): 424. Examples for ALL, including extrameduallary disease assessment such as cerebrospinal fluid cytology, can be found in Cheson, et al. Revised recommendations of the International Working Group for Diagnosis, Standardization of Response Criteria, Treatment Outcomes, and Reporting Standards for Therapeutic Trials in Acute Myeloid Leukemia. J Clin Oncol. 2003; 21(24):4642-9. Examples for BPDCN, can be found in Cheson et al. Report of an international workshop to standardize response criteria for non-Hodgkin's lymphomas. NCI Sponsored International Working Group. 1999; 17(4):1244; Cheson et al. Journal of Clinical Oncology. 2007; 25(5):579-86; Olsen et al. Journal of Clinical Oncology. 2011; 29(18):2598-607; Pagano et al. Haematologica. 2013; 98(2):239-46. Examples for chronic myeloid leukemia in blast phase can be found in Cortes et al. Blood. 2007; 109(8):3207-13.

Improvement in one or more symptoms associated with a CD123-expressing cancer include feeling less tired, feeling less weak, feeling less dizzy or lightheaded, reduction in shortness of breath, reduction in fever, fewer infections, quicker recovery from infections, reduction in ease of bruising, reduction in bleeding episodes, weight gain, reduction in night sweats, gain of appetite, reduction in abdominal swelling, reduction in lymph node swelling, reduction in bone or joint pain, and reduction in thymus swelling.

An improvement in the CD123-expressing cancer can be characterized as a “complete remission” or “complete response”. The terms “complete remission” or “complete response” in relation to cancer can mean all signs and/or symptoms of cancer have disappeared, although in some cases, a cancer patient may still have cancer cells in the body. Complete remission can result in an absence of clinically detectable disease with normalization of any previously abnormal radiographic studies, bone marrow, and cerebrospinal fluid (CSF). In one case, complete remission is defined as <5% bone marrow blasts, no circulating blasts or blasts with Auer rods, absence of extramedullary disease, and normalization of blood counts (absolute neutrophil count ≥1000/microliter and platelet count ≥100000/microliter). In some cases, with regarding to blood cancers such as AML and ALL, complete remission can, in addition to absence of morphologic evidence of leukemia, result in a recovery of normal blood cell counts to a normal range.

Alternatively, an improvement in the CD123-expressing cancer can be characterized as a “partial remission” or “partial response”. The term “partial remission” or “partial response” in relation to cancer can mean that some, but not all, signs and/or symptoms of cancer have disappeared. For example, in some cases, partial response can convey that at least about a 5% decrease in at least one measurable tumor burden (i.e., the number of malignant cells present in the subject, or the measured bulk of tumor masses or the quantity of abnormal monoclonal protein) in the absence of new lesions, which can persist for 4 to 8 weeks, or 6 to 8 weeks. In some cases, partial response can lead to at least about a 10% decrease in at least one measurable tumor burden. In some cases, partial response mean at least about a 15% decrease in at least one measurable tumor burden. In some cases, partial response mean at least about a 20% decrease in at least one measurable tumor burden. In some cases, partial response mean at least about a 25% decrease in at least one measurable tumor burden. In some cases, partial response mean at least about a 30% decrease in at least one measurable tumor burden. In some cases, partial response mean at least about a 35% decrease in at least one measurable tumor burden. In some cases, partial response mean at least about a 40% decrease in at least one measurable tumor burden. In some cases, partial response mean at least about a 45% decrease in at least one measurable tumor burden. In some cases, partial response mean at least about a 50% decrease in at least one measurable tumor burden. In some cases, partial response mean at least about a 60% decrease in at least one measurable tumor burden. In some cases, partial response mean at least about a 70% decrease in at least one measurable tumor burden. In some cases, partial response mean at least about a 80% decrease in at least one measurable tumor burden. In some cases, partial response mean at least about a 90% decrease in at least one measurable tumor burden.

### II. Overview

Disclosed herein are methods of treating a cancer that include cells expressing CD123 (“CD123-expressing cancer”), for example, a hematologic cancer, such as leukemia, through the administration of certain bispecific anti-CD123×anti-CD3 antibodies at particular dosages. The term “CD123-expressing cancer” can refer to a cancer that expresses CD123 or a cancer that overexpresses CD123. The present invention also provides methods of combination therapies, for example, methods of treating a cancer that include cells expressing CD123 (“CD123-expressing cancer”), e.g., a hematologic cancer, such as leukemia, through the administration of certain bispecific anti-CD123×anti-CD3 antibodies (e.g., XmAb14045) in combination with one or more therapies that can ameliorate side effects of an anti-CD123×anti-CD3 bispecific antibody.

### III. Antibodies

The present invention is directed to the administration of bispecific antibodies, such as anti-CD123×anti-CD3 antibodies, for the treatment of CD123-expressing cancers, such as particular leukemias. For example, some embodiments of antibodies with bispecific formats of the figures and polynucleotide/polypeptide sequences, are disclosed in U.S. Pat. Appl. Pub. No. 2016/0229924.

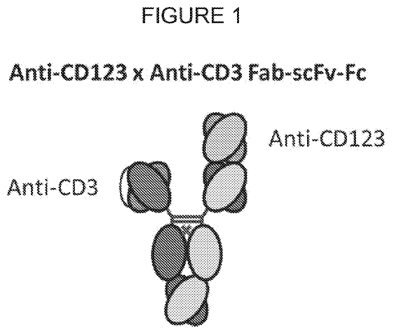

In some embodiments, the bispecific anti-CD123×anti-CD3 antibodies have a “bottle opener” format as is generally depicted in FIG. 1. In this embodiment, the anti-CD3 antigen binding domain is the scFv-Fc domain monomer and the anti-CD123 antigen binding domain is the Fab monomer (see e.g., U.S. Pat. Appl. Pub. Nos. 2014/0288275; 2014/0294823; and 2016/0355608).

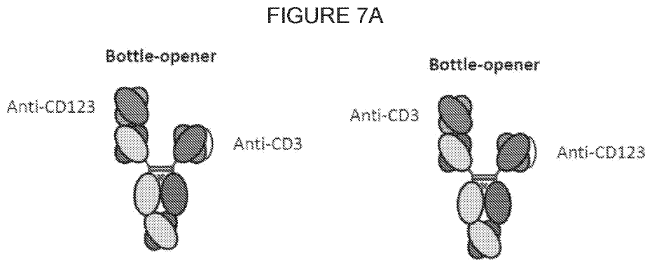

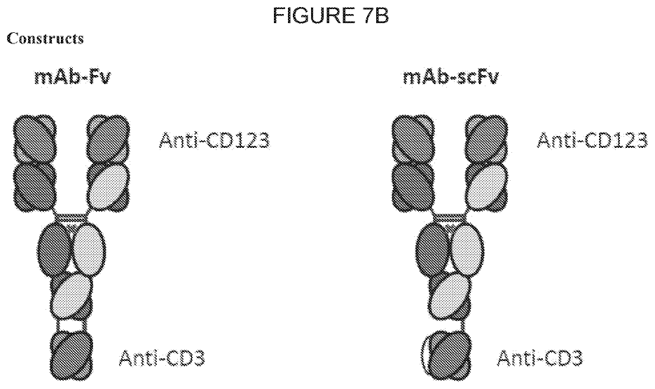

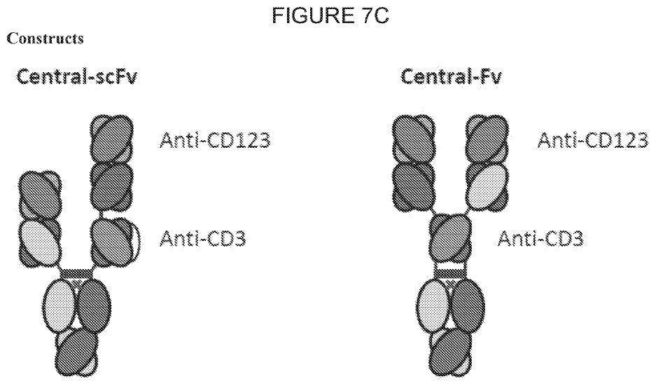

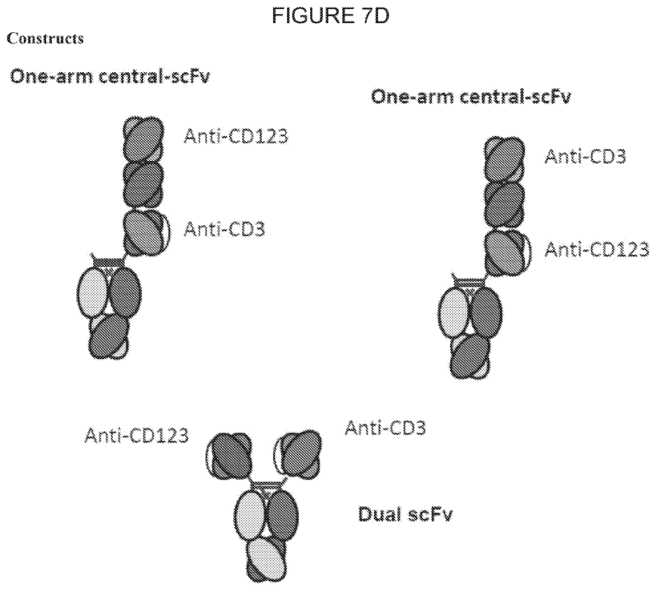

Alternate formats for the bispecific, heterodimeric anti-CD123×anti-CD3 antibodies are shown in FIG. 7, which also generally rely on the use of Fabs and scFv domains in different formats.

In addition, other heterodimeric and non-heterodimeric anti-CD123×anti-CD3 bispecific antibodies, can be dosed at the same dosage levels and by the same methods as described therein.

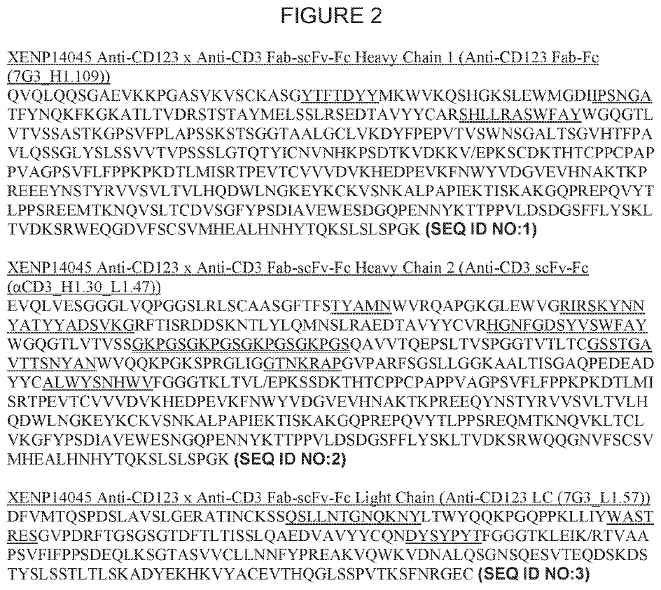

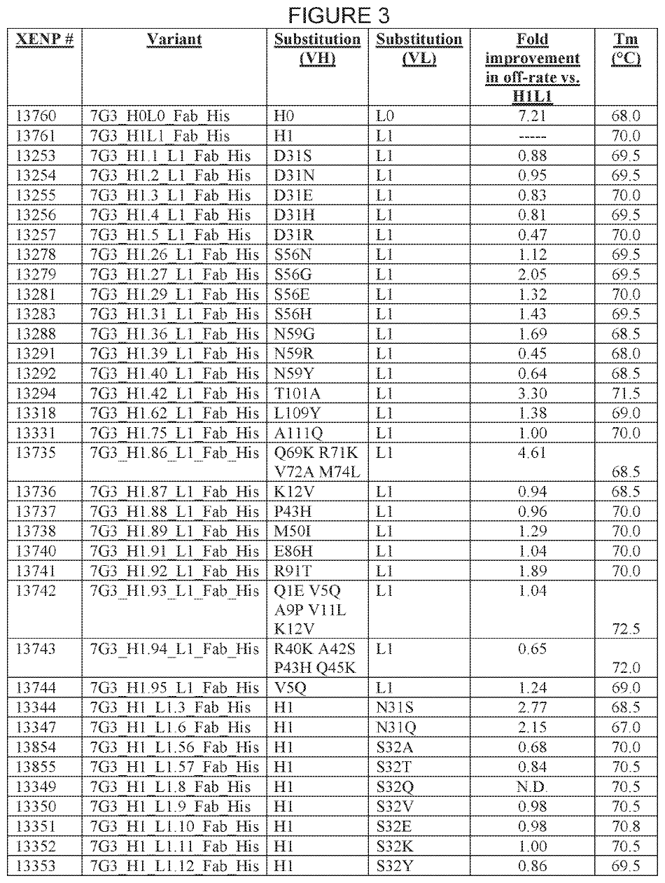

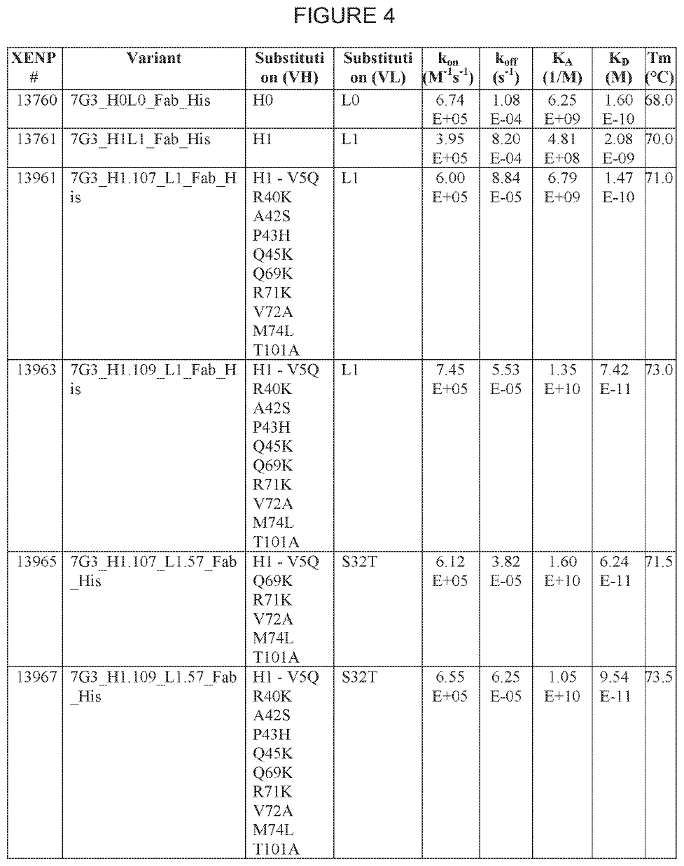

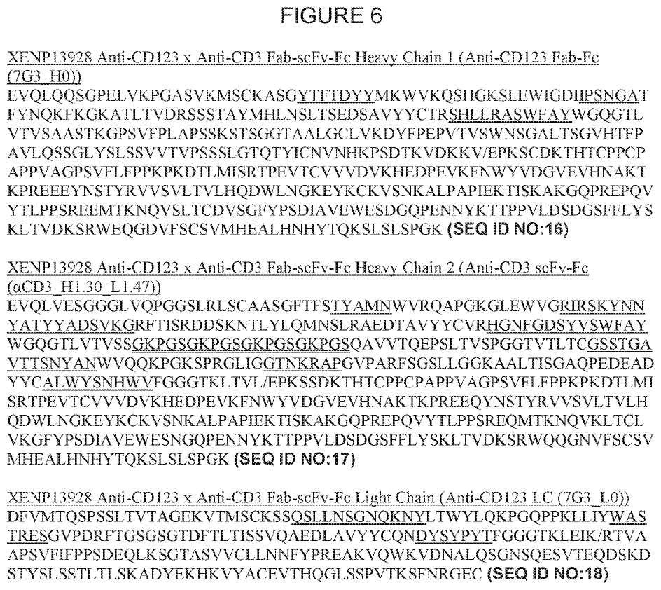

The anti-CD3 scFv antigen binding domain can have the sequence depicted in FIG. 2, or can be selected from the group consisting of:
1) the set of 6 CDRs (vhCDR1, vhCDR2, vhCDR3, vlCDR1, vlCDR2 and vlCDR3) from any one of the anti-CD3 antigen binding domain sequence depicted in FIGS. 2 and 6 of U.S. Pat. Appl. Pub. No. 2014/0288275;
2) the variable heavy and variable light chains from any one of the anti-CD3 antigen binding domain sequence depicted in FIGS. 2 and 6 of U.S. Pat. Appl. Pub. No. 2014/0288275;
3) the scFv domains from any one of the anti-CD3 scFv sequence depicted in FIG. 2 of U.S. Pat. Appl. Pub. No. 2014/0288275;
4) other known anti-CD3 variable heavy and variable light chains, that can be combined to form scFvs (or Fabs, when the format is reversed or an alternative format is used); and
5) any one of the anti-CD3 antigen binding domains of FIGS. 2, 3, 4, 5, 6, and 7 of U.S. Pat. Appl. Pub. No. 2016/0229924.

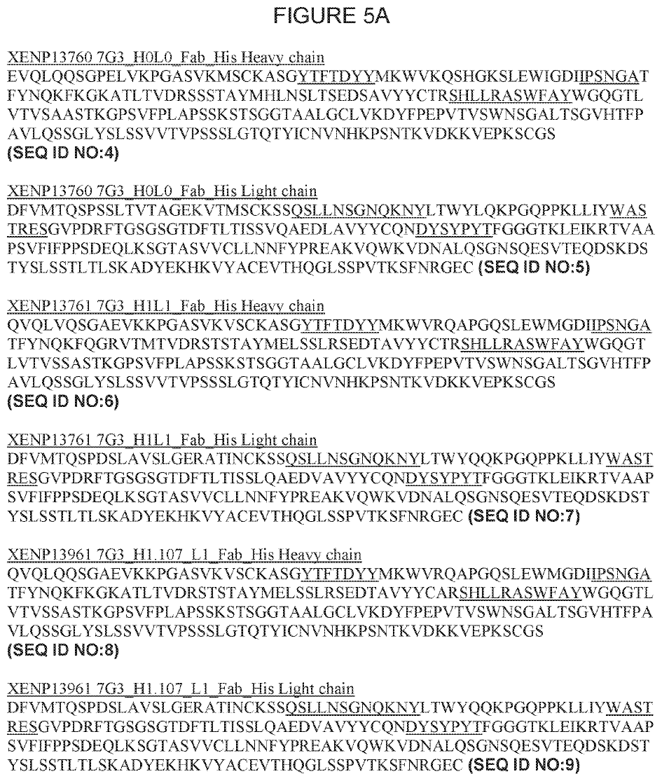

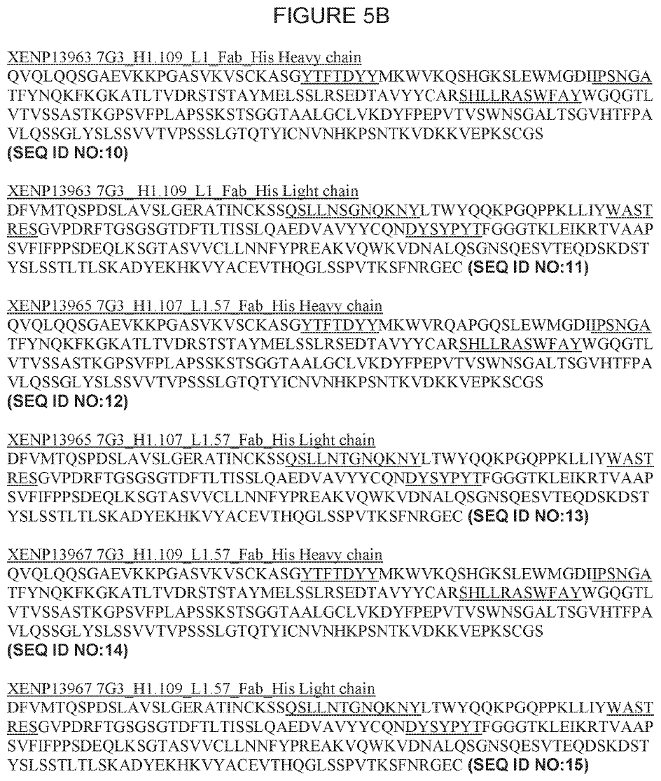

The anti-CD123 Fab binding domain can have the sequence depicted in FIG. 2 or 5, or can be selected from the group consisting of:
1) The set of 6 CDRs (vhCDR1, vhCDR2, vhCDR3, vlCDR1, vlCDR2 and vlCDR3) from any one of the anti-CD123 antigen binding domain sequence depicted in U.S. Pat. Appl. Pub. No. 2016/0229924, including those depicted in FIGS. 2, 3 and 12;
2) The variable heavy and variable light chains from any one of the anti-CD123 antigen binding domain sequence depicted in U.S. Pat. Appl. Pub. No. 2016/0229924, including those depicted in FIGS. 2, 3 and 12; and
3) Other anti-CD123 variable heavy and variable light chains as are also known, that can be combined to form Fabs (or scFvs, when the format is reversed or an alternative format is used).

One bispecific antibody that can be used in the dosing regimens described throughout is XmAb14045 as shown in FIG. 2. The XmAb14045 bispecific antibody includes a first monomer comprising SEQ ID NO: 1, a second monomer comprising SEQ ID NO: 2, and a light chain comprising SEQ ID NO: 3.

The bispecific anti-CD123×anti-CD3 antibodies as used throughout can be made through known methods. The disclosure further provides polynucleotide compositions encoding the bispecific anti-CD123×anti-CD3 antibodies. Further, the polynucleotide compositions will depend on the format and scaffold of the bispecific anti-CD123×anti-CD3 antibodies. Thus, for example, when the format requires three amino acid sequences, such as for the triple F format (e.g. a first amino acid monomer comprising an Fc domain and a scFv, a second amino acid monomer comprising a heavy chain and a light chain), three polynucleotides can be incorporated into one or more vectors for expression. Similarly, some formats (e.g. dual scFv formats such as disclosed in FIG. 7) only two polynucleotides are needed; they can also be put into one or two expression vectors.

The polynucleotides encoding the components of the bispecific antibodies can be incorporated into expression vectors, and depending on the host cells can be used to produce the bispecific anti-CD123×anti-CD3 antibodies. Generally the polynucleotides are operably linked to any number of regulatory elements (promoters, origin of replication, selectable markers, ribosomal binding sites, inducers, etc.). The expression vectors can be extra-chromosomal or integrating vectors.

The polynucleotides and/or expression vectors are then transformed into any number of different types of host cells, including but not limited to mammalian, bacterial, yeast, insect and/or fungal cells, with mammalian cells (e.g. CHO cells).

In some embodiments, polynucleotides encoding each monomer and the optional polynucleotides encoding a light chain, as applicable depending on the format, are each contained within a single expression vector, controlled using different or the same promoter. In some embodiments, each of these two or three polynucleotides are contained on a different expression vector.

The heterodimeric bispecific anti-CD123×anti-CD3 antibodies are made by culturing host cells comprising expression vector(s). Once produced, traditional antibody purification steps are performed, such as an ion exchange chromatography step. As discussed in U.S. Pat. No. 9,650,446 and Int. Publ. No. WO2014/145806, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the “triple F” heterodimer (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

Once made, the bispecific anti-CD123×anti-CD3 antibodies are administered to human subjects in dosages as outlined herein.

### IV. Pharmaceutical Compositions and Pharmaceutical Administration

XmAb14045 can be incorporated into pharmaceutical compositions suitable for administration to a human subject according to a dosage regimen described herein. As used herein, “dosage regimen” refers to a systematic plan of drug administration regarding formulation, route of administration, drug dose, dosing interval and treatment duration. Typically, the pharmaceutical composition comprises XmAb14045 and a pharmaceutically acceptable carrier. As used herein, “pharmaceutically acceptable carrier” includes any and all solvents, dispersion media, coatings, isotonic and absorption delaying agents, and the like that are physiologically compatible and are suitable for administration to a subject for the methods described herein. Examples of pharmaceutically acceptable carriers include one or more of water, saline, phosphate buffered saline, dextrose, glycerol, ethanol and the like, as well as any combination thereof. In some cases, isotonic agents can be included, for example, sugars, polyalcohols such as mannitol, sorbitol, or sodium chloride in the composition. Pharmaceutically acceptable carriers may further comprise minor amounts of auxiliary substances such as surfactants (such as nonionic surfactants) wetting or emulsifying agents (such as a polysorbate), preservatives or buffers (such as an organic acid, which as a citrate or an acetate), which enhance the shelf life or effectiveness of XmAb14045. Examples of pharmaceutically acceptable carriers include polysorbates (polysorbate-80).

In one embodiment, the pharmaceutical composition comprises XmAb14045 and a preservative or buffer. In one embodiment, the pharmaceutical composition comprises XmAb14045 and histidine. In one embodiment, the pharmaceutical composition comprises XmAb14045 and an acetate. In one embodiment, the pharmaceutical composition comprises XmAb14045 and sodium acetate. In one embodiment, the pharmaceutical composition comprises XmAb14045 and a citrate. In one embodiment, the pharmaceutical composition comprises XmAb14045 and sodium citrate.

In one embodiment, the pharmaceutical composition comprises XmAb14045 and an isotonic agent. In one embodiment, the pharmaceutical composition comprises XmAb14045 and a polyalcohol. In one embodiment, the pharmaceutical composition comprises XmAb14045 and mannitol. In one embodiment, the pharmaceutical composition comprises XmAb14045 and sorbitol. In one embodiment, the pharmaceutical composition comprises XmAb14045 and sodium chloride. In one embodiment, the pharmaceutical composition comprises XmAb14045 and potassium chloride.

In one embodiment, the pharmaceutical composition comprises XmAb14045 and a wetting or emulsifying agent. In one embodiment, the pharmaceutical composition comprises XmAb14045 and a polysorbate. In one embodiment, the pharmaceutical composition comprises XmAb14045 and polysorbate-80.

In one embodiment, the pharmaceutical composition comprises XmAb14045 and an intravenous solution stabilizer. In one embodiment, the intravenous solution stabilizer comprises a polysorbate and a citrate. In one embodiment, the pharmaceutical composition comprises XmAb14045 and sodium citrate and polysorbate-80.

In one embodiment, the pharmaceutical composition comprises XmAb14045 and a buffer and an isotonic agent. In one embodiment, the pharmaceutical composition comprises XmAb14045 and a buffer and sorbitol. In one embodiment, the pharmaceutical composition comprises XmAb14045 and an acetate and an isotonic agent. In one embodiment, the pharmaceutical composition comprises XmAb14045 and histidine and an isotonic agent. In one embodiment, the pharmaceutical composition comprises XmAb14045 and an acetate and sorbitol. In one embodiment, the pharmaceutical composition comprises XmAb14045 and sodium acetate and sorbitol. In one embodiment, the pharmaceutical composition comprises XmAb14045 and histidine and sorbitol.

In one embodiment, the pharmaceutical composition comprises XmAb14045 and a buffer and an isotonic agent and an intravenous solution stabilizer. In one embodiment, the pharmaceutical composition comprises XmAb14045 and a buffer and sorbitol and an intravenous solution stabilizer. In one embodiment, the pharmaceutical composition comprises XmAb14045 and an acetate and an isotonic agent and an intravenous solution stabilizer. In one embodiment, the pharmaceutical composition comprises XmAb14045 and histidine and an isotonic agent and an intravenous solution stabilizer. In one embodiment, the pharmaceutical composition comprises XmAb14045 and an acetate and sorbitol and an intravenous solution stabilizer. In one embodiment, the pharmaceutical composition comprises XmAb14045 and sodium acetate and sorbitol and an intravenous solution stabilizer. In one embodiment, the pharmaceutical composition comprises XmAb14045 and histidine and sorbitol and an intravenous solution stabilizer.

In one embodiment, the pharmaceutical composition comprises XmAb14045 and sodium chloride. In one embodiment, the pharmaceutical composition comprises XmAb14045 and sodium chloride and polysorbate-80. In one embodiment, the pharmaceutical composition comprises XmAb14045 and sodium citrate and sodium chloride. In one embodiment, the pharmaceutical composition comprises XmAb14045 and sodium citrate, sodium chloride, and polysorbate-80. In one embodiment, the pharmaceutical composition comprises XmAb14045 and sodium citrate, sodium chloride, sodium acetate, sorbitol and polysorbate-80. In one embodiment, the pharmaceutical composition comprises XmAb14045 and sodium citrate, sodium chloride, histidine, sorbitol and polysorbate-80.

The pharmaceutical compositions can be in a variety of forms. These include, for example, liquid, semi-solid and solid dosage forms, such as liquid solutions (e.g., injectable and infusible solutions), dispersions or suspensions. The form depends on the intended mode of administration and therapeutic application. Exemplary compositions are in the form of injectable or infusible solutions, such as compositions similar to those used for passive immunization of humans with other antibodies. In one embodiment, the mode of administration is intravenous. In one embodiment, the antibody is administered by intravenous infusion or injection.

Pharmaceutical compositions typically must be sterile and stable under the conditions of manufacture and storage. Sterile injectable solutions can be prepared by incorporating the antibody in the required amount in an appropriate solvent with one or any combination of ingredients enumerated herein, as required, followed by filtered sterilization. Generally, dispersions are prepared by incorporating the antibody into a sterile vehicle that contains a basic dispersion medium and the required other ingredients from those enumerated herein.

XmAb14045 can be administered by any known method. In one embodiment, the route/mode of administration is intravenous injection. The route and/or mode of administration can vary depending upon the desired results.

### V. CD123-Expressing Cancer Treatment Protocols

In one embodiment, the antibodies of the invention treat a CD123-expressing cancer. In one embodiment, the CD123-expressing cancer is a hematologic cancer. In one embodiment, the CD123-expressing cancer is a leukemia.

CD123 is frequently expressed on hematologic malignancies, such as 96-98% of acute myelogenous leukemia cases; >50% of myelodysplastic syndrome cases; 82-100% of B-cell acute lymphoblastic leukemia cases; 83-100% of Blastic plasmacytoid dendritic cell neoplasm cases; 75-100% of Chronic myelogenous leukemia cases; and 95-100% of Hairy cell leukemia cases.

Leukemia is a cancer of the blood or bone marrow characterized by an abnormal increase of blood cells, usually leukocytes (white blood cells). Leukemia is a broad term covering a spectrum of diseases. The first division is between its acute and chronic forms: (i) acute leukemia is characterized by the rapid increase of immature blood cells. This crowding makes the bone marrow unable to produce healthy blood cells. Immediate treatment is required in acute leukemia due to the rapid progression and accumulation of the malignant cells, which then spill over into the bloodstream and spread to other organs of the body. Acute forms of leukemia are the most common forms of leukemia in children; (ii) chronic leukemia is distinguished by the excessive buildup of relatively mature, but still abnormal, white blood cells. Typically taking months or years to progress, the cells are produced at a much higher rate than normal cells, resulting in many abnormal white blood cells in the blood. Chronic leukemia mostly occurs in older people, but can theoretically occur in any age group. Additionally, the diseases are subdivided according to which kind of blood cell is affected. This split divides leukemias into lymphoblastic or lymphocytic leukemias and myeloid or myelogenous leukemias: (i) lymphoblastic or lymphocytic leukemias, the cancerous change takes place in a type of marrow cell that normally goes on to form lymphocytes, which are infection-fighting immune system cells; (ii) myeloid or myelogenous leukemias, the cancerous change takes place in a type of marrow cell that normally goes on to form red blood cells, some other types of white cells, and platelets.

In one embodiment, the leukemia is selected from the group consisting of acute lymphocytic leukemia (ALL), acute myeloid leukemia (AML), chronic myeloid leukemia (CML), hairy cell leukemia (HCL), and blastic plasmacytoid dendritic cell neoplasm (BPDCN). In one embodiment, the leukemia is acute lymphocytic leukemia (ALL). In one embodiment, the leukemia is myelodysplastic syndrome. In one embodiment, the leukemia is acute myeloid leukemia (AML). In one embodiment, the leukemia is chronic myeloid leukemia (CML). In one embodiment, the leukemia is chronic phase chronic myeloid leukemia. In one embodiment, the leukemia is accelerated phase chronic myeloid leukemia. In one embodiment, the leukemia is blast phase chronic myeloid leukemia. In one embodiment, the leukemia is hairy cell leukemia (HCL). In one embodiment, the leukemia is classic hairy cell leukemia (HCLc). In one embodiment, the leukemia is acute myeloid leukemia (AML), where the AML is primary acute myeloid leukemia. In one embodiment, the leukemia is acute myeloid leukemia (AML), where the AML is secondary acute myeloid leukemia. In one embodiment, the leukemia is erythroleukemia. In one embodiment, the leukemia is eosinophilic leukemia. In one embodiment, the leukemia is acute myeloid leukemia (AML), where the AML does not include acute promyelocytic leukemia. In one embodiment, the leukemia is acute myeloid leukemia (AML), where the AML is blastic plasmacytoid dendritic cell neoplasm. In one embodiment, the leukemia is B-cell acute lymphocytic leukemia (B-ALL). In one embodiment, the leukemia is T-cell acute lymphocytic leukemia (T-ALL).

In one embodiment, the leukemia is relapsed acute myeloid leukemia (AML). In one embodiment, the leukemia is refractory acute myeloid leukemia (AML).

In some embodiments, the cancer is treated according to a method described herein. In one embodiment, the cancer is treated by dispensing XmAb14045 to the human subject in one or more phases. Each phase comprises dose(s) of XmAb14045 provided on a per week or per month basis (‘dosage regimen’). Each phase can last for one or more weeks or months, or until remission. In one embodiment, the antibody is administered until partial remission. In one embodiment, the antibody is administered until complete remission.

In one embodiment, the method of treatment comprises an antibody being dispensed in one to four phases. In one embodiment, a phase has the same dosage regimen that occurs between one (1) and twenty (20) times, or until remission). In one embodiment, the dosage regimen has a dose amount (quantity of an antibody) and an administration time (the length of time in which the dose amount is administered).

In one embodiment, the method comprises a first phase. In one embodiment, the method comprises a first phase. In one embodiment, the method comprises a first phase and a second phase. In one embodiment, the method comprises a first phase and a second phase, where each phase is different. In one embodiment, the method comprises a first phase and a second phase, where each phase is different. In one embodiment, the method comprises a first phase and a second phase and a third phase. In one embodiment, the method comprises a first phase and a second phase and a third phase. In one embodiment, the method comprises a first phase and a second phase and a third phase, where each phase is different. In one embodiment, the method comprises a first phase and a second phase and a third phase and a fourth phase. In one embodiment, the method comprises a first phase and a second phase and a third phase and a fourth phase. In one embodiment, the method comprises a first phase and a second phase and a third phase and a fourth phase, where each phase is different. In one embodiment, the method comprises a first phase and a second phase and a third phase and a fourth phase and a fifth phase. In one embodiment, the method comprises a first phase and a second phase and a third phase and a fourth phase and a fifth phase. In one embodiment, the method comprises a first phase and a second phase and a third phase and a fourth phase and a fifth phase, where each phase is different. In one embodiment, the method comprises a first phase and a second phase and a third phase and a fourth phase and a fifth phase and a sixth phase. In one embodiment, the method comprises a first phase and a second phase and a third phase and a fourth phase and a fifth phase and a sixth phase. In one embodiment, the method comprises a first phase and a second phase and a third phase and a fourth phase and a fifth phase and a sixth phase, where each phase is different. In one embodiment, the method comprises a first phase and a second phase and a third phase and a fourth phase and a fifth phase and a sixth phase and a seventh phase. In one embodiment, the method comprises a first phase and a second phase and a third phase and a fourth phase and a fifth phase and a sixth phase and a seventh phase. In one embodiment, the method comprises a first phase and a second phase and a third phase and a fourth phase and a fifth phase and a sixth phase and a seventh phase, where each phase is different.

### V. a). Dose

A dose has a specific amount of antibody that is administered to a human subject over a defined time period. The amount of antibody administered to a human subject is also known as the dose amount. The time over which the dose amount is administered to a human subject is also known as the administration time.

### V. a) i). Dose Amount

The dose amount may be determined or adjusted by measuring the amount of bispecific anti-CD123×anti-CD3 antibody (e.g., XmAb14045) in the blood upon administration, for instance taking out a biological sample and using anti-idiotypic antibodies which target the antigen binding region of the bispecific anti-CD123×anti-CD3 antibody (e.g., XmAb14045).

PARAGRAPH A includes the following dose amounts: In one embodiment, the dose amount is between about 3 ng/kg and about 750 ng/kg.

PARAGRAPH B includes any one of the following dose amounts: In one embodiment, the dose amount is between about 30 ng/kg and about 750 ng/kg. In one embodiment, the dose amount is between about 75 ng/kg and about 750 ng/kg.

PARAGRAPH C includes any one of the following dose amounts: In one embodiment, the dose amount is between about 1 ng/kg and about 5 ng/kg. In one embodiment, the dose amount is between about 2 ng/kg and about 4 ng/kg. In one embodiment, the amount is about 3 ng/kg. In one embodiment, the amount is 3 ng/kg.

PARAGRAPH D includes any one of the following dose amounts: In one embodiment, the dose amount is between about 1 ng/kg and about 20 ng/kg. In one embodiment, the dose amount is between about 5 ng/kg and about 15 ng/kg. In one embodiment, the dose amount is between about 7 ng/kg and about 13 ng/kg. In one embodiment, the dose amount is between about 9 ng/kg and about 11 ng/kg. In one embodiment, the dose amount is about 10 ng/kg. In one embodiment, the dose amount is 10 ng/kg.

PARAGRAPH E includes any one of the following dose amounts: In one embodiment, the dose amount is between about 10 ng/kg and about 50 ng/kg. In one embodiment, the dose amount is between about 20 ng/kg and about 40 ng/kg. In one embodiment, the dose amount is between about 25 ng/kg and about 35 ng/kg. In one embodiment, the dose amount is about 30 ng/kg. In one embodiment, the dose amount is 30 ng/kg.

PARAGRAPH F includes any one of the following dose amounts: In one embodiment, the dose amount is between about 25 ng/kg and about 150 ng/kg. In one embodiment, the dose amount is between about 50 ng/kg and about 125 ng/kg. In one embodiment, the dose amount is between about 75 ng/kg and about 125 ng/kg. In one embodiment, the dose amount is between about 90 ng/kg and about 120 ng/kg. In one embodiment, the dose amount is between about 100 ng/kg and about 110 ng/kg. In one embodiment, the dose amount is about 107 ng/kg. In one embodiment, the dose amount is between about 50 ng/kg and about 100 ng/kg. In one embodiment, the dose amount is between about 55 ng/kg and about 95 ng/kg. In one embodiment, the dose amount is between about 60 ng/kg and about 90 ng/kg. In one embodiment, the dose amount is between about 65 ng/kg and about 85 ng/kg. In one embodiment, the dose amount is between about 70 ng/kg and about 80 ng/kg. In one embodiment, the dose amount is about 75 ng/kg. In one embodiment, the dose amount is 75 ng/kg.

PARAGRAPH G includes any one of the following dose amounts: In one embodiment, the dose amount is between about 50 ng/kg and about 250 ng/kg. In one embodiment, the dose amount is between about 75 ng/kg and about 225 ng/kg. In one embodiment, the dose amount is between about 100 ng/kg and about 200 ng/kg. In one embodiment, the dose amount is between about 100 ng/kg and about 175 ng/kg. In one embodiment, the dose amount is between about 100 ng/kg and about 150 ng/kg. In one embodiment, the dose amount is between about 110 ng/kg and about 135 ng/kg. In one embodiment, the dose amount is between about 120 ng/kg and about 130 ng/kg. In one embodiment, the dose amount is about 125 ng/kg. In one embodiment, the dose amount is between about 150 ng/kg and about 200 ng/kg. In one embodiment, the dose amount is between about 175 ng/kg and about 200 ng/kg. In one embodiment, the dose amount is between about 180 ng/kg and about 190 ng/kg. In one embodiment, the dose amount is about 185 ng/kg. In one embodiment, the dose amount is about 185 ng/kg. In one embodiment, the dose amount is about 188 ng/kg. In one embodiment, the dose amount is about 188 ng/kg. In one embodiment, the dose amount is 125 ng/kg. In one embodiment, the dose amount is between about 125 ng/kg and about 175 ng/kg. In one embodiment, the dose amount is about 150 ng/kg. In one embodiment, the dose amount is 150 ng/kg.

PARAGRAPH H includes any one of the following dose amounts: In one embodiment, the dose amount is between about 100 ng/kg and about 500 ng/kg. In one embodiment, the dose amount is between about 200 ng/kg and about 400 ng/kg. In one embodiment, the dose amount is between about 175 ng/kg and about 225 ng/kg. In one embodiment, the dose amount is between about 210 ng/kg and about 220 ng/kg. In one embodiment, the dose amount is about 217 ng/kg. In one embodiment, the dose amount is 217 ng/kg. In one embodiment, the dose amount is between about 225 ng/kg and about 275 ng/kg. In one embodiment, the dose amount is between about 240 ng/kg and about 260 ng/kg. In one embodiment, the dose amount is about 250 ng/kg. In one embodiment, the dose amount is 250 ng/kg. In one embodiment, the dose amount is between about 225 ng/kg and about 275 ng/kg. In one embodiment, the dose amount is between about 250 ng/kg and about 270 ng/kg. In one embodiment, the dose amount is about 260 ng/kg. In one embodiment, the dose amount is 260 ng/kg. In one embodiment, the dose amount is between about 300 ng/kg and about 350 ng/kg. In one embodiment, the dose amount is between about 320 ng/kg and about 330 ng/kg. In one embodiment, the dose amount is about 325 ng/kg. In one embodiment, the dose amount is 325 ng/kg. In one embodiment, the dose amount is between about 300 ng/kg and about 350 ng/kg. In one embodiment, the dose amount is between about 325 ng/kg and about 335 ng/kg. In one embodiment, the dose amount is about 330 ng/kg. In one embodiment, the dose amount is 330 ng/kg. In one embodiment, the dose amount is between about 350 ng/kg and about 400 ng/kg. In one embodiment, the dose amount is between about 370 ng/kg and about 380 ng/kg. In one embodiment, the dose amount is about 375 ng/kg. In one embodiment, the dose amount is 375 ng/kg. In one embodiment, the dose amount is between about 375 ng/kg and about 385 ng/kg. In one embodiment, the dose amount is about 383 ng/kg. In one embodiment, the dose amount is 383 ng/kg. In one embodiment, the dose amount is between about 225 ng/kg and about 375 ng/kg. In one embodiment, the dose amount is between about 250 ng/kg and about 350 ng/kg. In one embodiment, the dose amount is between about 275 ng/kg and about 325 ng/kg. In one embodiment, the dose amount is about 300 ng/kg. In one embodiment, the dose amount is 300 ng/kg. In one embodiment, the dose amount is between about 300 ng/kg and about 500 ng/kg. In one embodiment, the dose amount is between about 325 ng/kg and about 475 ng/kg. In one embodiment, the dose amount is between about 350 ng/kg and about 450 ng/kg. In one embodiment, the dose amount is between about 375 ng/kg and about 450 n/k. In one embodiment, the dose amount is between about 400 ng/kg and about 450 ng/kg. In one embodiment, the dose amount is between about 425 ng/kg and about 450 ng/kg. In one embodiment, the dose amount is between about 420 ng/kg and about 440 ng/kg. In one embodiment, the dose amount is about 430 ng/kg. In one embodiment, the dose amount is 430 ng/kg. In one embodiment, the dose amount is about 433 ng/kg. In one embodiment, the dose amount is 433 ng/kg.

PARAGRAPH I includes any one of the following dose amounts: In one embodiment, the dose amount is between about 350 ng/kg and about 650 ng/kg. In one embodiment, the dose amount is between about 400 ng/kg and about 600 ng/kg. In one embodiment, the dose amount is between about 400 ng/kg and about 500 ng/kg. In one embodiment, the dose amount is between about 425 ng/kg and about 475 ng/kg. In one embodiment, the dose amount is between about 450 ng/kg and about 470 ng/kg. In one embodiment, the dose amount is about 460 ng/kg. In one embodiment, the dose amount is 460 ng/kg. In one embodiment, the dose amount is between about 525 ng/kg and about 600 ng/kg. In one embodiment, the dose amount is between about 550 ng/kg and about 600 ng/kg. In one embodiment, the dose amount is between about 560 ng/kg and about 580 ng/kg. In one embodiment, the dose amount is about 570 ng/kg. In one embodiment, the dose amount is 570 ng/kg. In one embodiment, the dose amount is about 575 ng/kg. In one embodiment, the dose amount is 575 ng/kg. In one embodiment, the dose amount is between about 450 ng/kg and about 550 ng/kg. In one embodiment, the dose amount is between about 475 ng/kg and about 525 ng/kg. In one embodiment, the dose amount is about 500 ng/kg. In one embodiment, the dose amount is 500 ng/kg.

PARAGRAPH J includes any one of the following dose amounts: In one embodiment, the dose amount is between about 600 ng/kg and about 900 ng/kg. In one embodiment, the dose amount is between about 100 ng/kg and about 750 ng/kg. In one embodiment, the dose amount is between about 500 ng/kg and about 750 ng/kg. In one embodiment, the dose amount is between about 600 ng/kg and about 750 ng/kg. In one embodiment, the dose amount is between about 700 ng/kg and about 750 ng/kg. In one embodiment, the dose amount is between about 600 ng/kg and about 700 ng/kg. In one embodiment, the dose amount is between about 625 ng/kg and about 675 ng/kg. In one embodiment, the dose amount is between about 640 ng/kg and about 660 ng/kg. In one embodiment, the dose amount is about 650 ng/kg. In one embodiment, the dose amount is 650 ng/kg. In one embodiment, the dose amount is between about 650 ng/kg and about 700 ng/kg. In one embodiment, the dose amount is between about 660 ng/kg and about 680 ng/kg. In one embodiment, the dose amount is about 667 ng/kg. In one embodiment, the dose amount is 667 ng/kg. In one embodiment, the dose amount is between about 725 ng/kg and about 775 ng/kg. In one embodiment, the dose amount is between about 740 ng/kg and about 780 ng/kg. In one embodiment, the dose amount is between about 760 ng/kg and about 780 ng/kg. In one embodiment, the dose amount is between about 750 ng/kg and about 780 ng/kg. In one embodiment, the dose amount is about 767 ng/kg. In one embodiment, the dose amount is 767 ng/kg. In one embodiment, the dose amount is about 770 ng/kg. In one embodiment, the dose amount is 770 ng/kg. In one embodiment, the dose amount is between about 700 ng/kg and about 900 ng/kg. In one embodiment, the dose amount is between about 750 ng/kg and about 850 ng/kg. In one embodiment, the dose amount is between about 775 ng/kg and about 825 ng/kg. In one embodiment, the dose amount is about 800 ng/kg. In one embodiment, the dose amount is 800 ng/kg. In one embodiment, the dose amount is between about 650 ng/kg and about 850 ng/kg. In one embodiment, the dose amount is between about 700 ng/kg and about 800 ng/kg. In one embodiment, the dose amount is between about 725 ng/kg and about 775 ng/kg. In one embodiment, the dose amount is between about 740 ng/kg and about 760 ng/kg. In one embodiment, the dose amount is about 750 ng/kg. In one embodiment, the dose amount is 750 ng/kg.

PARAGRAPH K includes any one of the following dose amounts: In one embodiment, the dose amount is between about 700 ng/kg and about 1,900 ng/kg. In one embodiment, the dose amount is between about 1,500 ng/kg and about 1,900 ng/kg. In one embodiment, the dose amount is between about 1,300 ng/kg and about 1,500 ng/kg. In one embodiment, the dose amount is between about 1,350 ng/kg and about 1,450 ng/kg. In one embodiment, the dose amount is about 1,400 ng/kg. In one embodiment, the dose amount is 1,400 ng/kg. In one embodiment, the dose amount is between about 300 ng/kg and about 1,100 ng/kg. In one embodiment, the dose amount is between about 700 ng/kg and about 1,100 ng/kg. In one embodiment, the dose amount is between about 900 ng/kg and about 1,100 ng/kg. In one embodiment, the dose amount is between about 950 ng/kg and about 1,050 ng/kg. In one embodiment, the dose amount is about 1,000 ng/kg. In one embodiment, the dose amount is 1,000 ng/kg. In one embodiment, the dose amount is between about 1,100 ng/kg and about 1,200 ng/kg. In one embodiment, the dose amount is between about 1,125 ng/kg and about 1,175 ng/kg. In one embodiment, the dose amount is about 1,125 ng/kg. In one embodiment, the dose amount is 1,125 ng/kg. In one embodiment, the dose amount is about 1,150 ng/kg. In one embodiment, the dose amount is 1,150 ng/kg. In one embodiment, the dose amount is between about 1,150 ng/kg and about 1,180 ng/kg. In one embodiment, the dose amount is between about 1,160 ng/kg and about 1,175 ng/kg. In one embodiment, the dose amount is about 1,167 ng/kg. In one embodiment, the dose amount is 1,167 ng/kg. In one embodiment, the dose amount is between about 800 ng/kg and about 1,100 ng/kg. In one embodiment, the dose amount is between about 900 ng/kg and about 1,050 ng/kg. In one embodiment, the dose amount is between about 950 ng/kg and about 1,100 ng/kg. In one embodiment, the dose amount is between about 850 ng/kg and about 1,750 ng/kg. In one embodiment, the dose amount is between about 1,000 ng/kg and about 1,600 ng/kg. In one embodiment, the dose amount is between about 1,000 ng/kg and about 1,400 ng/kg. In one embodiment, the dose amount is between about 1,150 ng/kg and about 1,450 ng/kg. In one embodiment, the dose amount is between about 1,300 ng/kg and about 1,350 ng/kg. In one embodiment, the dose amount is about 1,333 ng/kg. In one embodiment, the dose amount is 1,333 ng/kg. In one embodiment, the dose amount is about 1,300 ng/kg. In one embodiment, the dose amount is 1,300 ng/kg.

PARAGRAPH L includes any one of the following dose amounts: In one embodiment, the amount is between about 900 ng/kg and about 3,400 ng/kg. In one embodiment, the amount is between about 1,200 ng/kg and about 3,400 ng/kg. In one embodiment, the amount is between about 1,400 ng/kg and about 2,400 ng/kg. In one embodiment, the amount is between about 1,500 ng/kg and about 1,800 ng/kg. In one embodiment, the amount is between about 1,500 ng/kg and about 1,900 ng/kg. In one embodiment, the amount is between about 1,700 ng/kg and about 1,800 ng/kg. In one embodiment, the dose amount is about 1,750 ng/kg. In one embodiment, the dose amount is 1,750 ng/kg. In one embodiment, the amount is between about 1,700 ng/kg and about 1,740 ng/kg. In one embodiment, the amount is between about 1,700 ng/kg and about 1,725 ng/kg. In one embodiment, the dose amount is about 1,714 ng/kg. In one embodiment, the dose amount is 1,714 ng/kg. In one embodiment, the amount is between about 1,400 ng/kg and about 3,200 ng/kg. In one embodiment, the amount is between about 1,600 ng/kg and about 3,000 ng/kg. In one embodiment, the dose amount is between about 1,800 ng/kg and about 2,200 ng/kg. In one embodiment, the dose amount is between about 1,900 ng/kg and about 2,100 ng/kg. In one embodiment, the dose amount is about 2,000 ng/kg. In one embodiment, the dose amount is 2,000 ng/kg. In one embodiment, the dose amount is between about 1,800 ng/kg and about 2,800 ng/kg. In one embodiment, the dose amount is between about 2,000 ng/kg and about 2,600 ng/kg. In one embodiment, the dose amount is between about 2,250 ng/kg and about 2,500 ng/kg. In one embodiment, the dose amount is between about 2,300 ng/kg and about 2,350 ng/kg. In one embodiment, the dose amount is about 2,333 ng/kg. In one embodiment, the dose amount is 2,333 ng/kg. In one embodiment, the dose amount is about 2,400 ng/kg. In one embodiment, the dose amount is 2,400 ng/kg. In one embodiment, the dose amount is between about 2,200 ng/kg and about 2,400 ng/kg. In one embodiment, the dose amount is about 2,300 ng/kg. In one embodiment, the dose amount is 2,300 ng/kg.

PARAGRAPH M includes any one of the following dose amounts: In one embodiment, the dose amount is between about 2,000 ng/kg and about 5,000 ng/kg. In one embodiment, the dose amount is between about 2,000 ng/kg and about 4,000 ng/kg. In one embodiment, the dose amount is between about 3,000 ng/kg and about 4,000 ng/kg. In one embodiment, the dose amount is between about 3,250 ng/kg and about 3,750 ng/kg. In one embodiment, the dose amount is between about 3,400 ng/kg and about 3,600 ng/kg. In one embodiment, the dose amount is about 3,500 ng/kg. In one embodiment, the dose amount is 3,500 ng/kg. In one embodiment, the dose amount is between about 3,000 ng/kg and about 5,000 ng/kg. In one embodiment, the dose amount is between about 3,400 ng/kg and about 3,600 ng/kg. In one embodiment, the dose amount is about 3,500 ng/kg. In one embodiment, the dose amount is 3,500 ng/kg. In one embodiment, the dose amount is between about 2,500 ng/kg and about 3,500 ng/kg. In one embodiment, the dose amount is between about 2,750 ng/kg and about 3,250 ng/kg. In one embodiment, the dose amount is about 3,000 ng/kg. In one embodiment, the dose amount is 3,000 ng/kg. In one embodiment, the dose amount is between about 2,750 ng/kg and about 3,000 ng/kg. In one embodiment, the dose amount is between about 2,800 ng/kg and about 2,900 ng/kg. In one embodiment, the dose amount is between about 2,830 ng/kg and about 2,880 ng/kg. In one embodiment, the dose amount is about 2,857 ng/kg. In one embodiment, the dose amount is 2,857 ng/kg. In one embodiment, the dose amount is between about 3,200 ng/kg and about 3,400 ng/kg. In one embodiment, the dose amount is between about 3,300 ng/kg and about 3,350 ng/kg. In one embodiment, the dose amount is about 3,333 ng/kg. In one embodiment, the dose amount is 3,333 ng/kg. In one embodiment, the dose amount is between about 2,500 ng/kg and about 5,000 ng/kg. In one embodiment, the dose amount is between about 3,000 ng/kg and about 5,000 ng/kg. In one embodiment, the dose amount is between about 3,500 ng/kg and about 4,500 ng/kg. In one embodiment, the dose amount is between about 3,750 ng/kg and about 4,250 ng/kg. In one embodiment, the dose amount is about 4,000 ng/kg. In one embodiment, the dose amount is 4,000 ng/kg.

PARAGRAPH N includes any one of the following dose amounts: In one embodiment, the dose amount is between about 3,000 ng/kg and about 11,000 ng/kg. In one embodiment, the dose amount is between about 4,000 ng/kg and about 10,000 ng/kg. In one embodiment, the dose amount is between about 6,000 ng/kg and about 7,000 ng/kg. In one embodiment, the dose amount is between about 6,500 ng/kg and about 6,750 ng/kg. In one embodiment, the dose amount is about 6,667 ng/kg. In one embodiment, the dose amount is 6,667 ng/kg. In one embodiment, the dose amount is about 6,700 ng/kg. In one embodiment, the dose amount is 6,700 ng/kg. In one embodiment, the dose amount is between about 4,000 ng/kg and about 6,000 ng/kg. In one embodiment, the dose amount is between about 4,500 ng/kg and about 5,500 ng/kg. In one embodiment, the dose amount is between about 4,750 ng/kg and about 5,250 ng/kg. In one embodiment, the dose amount is between about 4,900 ng/kg and about 5,100 ng/kg. In one embodiment, the dose amount is about 5,000 ng/kg. In one embodiment, the dose amount is 5,000 ng/kg. In one embodiment, the dose amount is between about 4,000 ng/kg and about 8,000 ng/kg. In one embodiment, the dose amount is between about 5,000 ng/kg and about 7,000 ng/kg. In one embodiment, the dose amount is between about 5,500 ng/kg and about 6,000 ng/kg. In one embodiment, the dose amount is between about 5,750 ng/kg and about 5,900 ng/kg. In one embodiment, the dose amount is about 5,833 ng/kg. In one embodiment, the dose amount is 5,833 ng/kg. In one embodiment, the dose amount is between about 5,500 ng/kg and about 6,500 ng/kg. In one embodiment, the dose amount is between about 5,900 ng/kg and about 6,100 ng/kg. In one embodiment, the dose amount is about 6,000 ng/kg. In one embodiment, the dose amount is 6,000 ng/kg. In one embodiment, the dose amount is between about 5,000 ng/kg and about 9,000 ng/kg. In one embodiment, the dose amount is between about 6,000 ng/kg and about 8,000 ng/kg. In one embodiment, the dose amount is between about 6,500 ng/kg and about 7,500 ng/kg. In one embodiment, the dose amount is about 7,000 ng/kg. In one embodiment, the dose amount is 7,000 ng/kg.

PARAGRAPH O includes any one of the following dose amounts: In one embodiment, the dose amount is between about 7,000 ng/kg and about 17,000 ng/kg. In one embodiment, the dose amount is between about 8,000 ng/kg and about 16,000 ng/kg. In one embodiment, the dose amount is between about 8,000 ng/kg and about 14,000 ng/kg. In one embodiment, the dose amount is between about 8,000 ng/kg and about 12,000 ng/kg. In one embodiment, the dose amount is between about 9,000 ng/kg and about 11,000 ng/kg. In one embodiment, the dose amount is between about 9,500 ng/kg and about 10,500 ng/kg. In one embodiment, the dose amount is about 10,000 ng/kg. In one embodiment, the dose amount is 10,000 ng/kg. In one embodiment, the dose amount is between about 8,000 ng/kg and about 9,500 ng/kg. In one embodiment, the dose amount is between about 8,250 ng/kg and about 9,250 ng/kg. In one embodiment, the dose amount is between about 8,500 ng/kg and about 9,000 ng/kg. In one embodiment, the dose amount is about 8,750 ng/kg. In one embodiment, the dose amount is 8,750 ng/kg. In one embodiment, the dose amount is between about 9,000 ng/kg and about 15,000 ng/kg. In one embodiment, the dose amount is between about 10,000 ng/kg and about 14,000 ng/kg. In one embodiment, the dose amount is between about 11,250 ng/kg and about 12,500 ng/kg. In one embodiment, the dose amount is between about 11,250 ng/kg and about 12,000 ng/kg. In one embodiment, the dose amount is between about 11,500 ng/kg and about 11,750 ng/kg. In one embodiment, the dose amount is about 11,667 ng/kg. In one embodiment, the dose amount is 11,667 ng/kg. In one embodiment, the dose amount is about 11,700 ng/kg. In one embodiment, the dose amount is 11,700 ng/kg. In one embodiment, the dose amount is between about 11,000 ng/kg and about 13,000 ng/kg. In one embodiment, the dose amount is about 12,000 ng/kg. In one embodiment, the dose amount is 12,000 ng/kg.

PARAGRAPH P includes any one of the following dose amounts: In one embodiment, the dose amount is between about 12,000 ng/kg and about 28,000 ng/kg. In one embodiment, the dose amount is between about 14,000 ng/kg and about 26,000 ng/kg. In one embodiment, the dose amount is between about 16,000 ng/kg and about 24,000 ng/kg. In one embodiment, the dose amount is between about 16,000 ng/kg and about 20,000 ng/kg. In one embodiment, the dose amount is between about 17,000 ng/kg and about 19,000 ng/kg. In one embodiment, the dose amount is between about 17,000 ng/kg and about 18,000 ng/kg. In one embodiment, the dose amount is between about 17,250 ng/kg and about 17,750 ng/kg. In one embodiment, the dose amount is about 17,750 ng/kg. In one embodiment, the dose amount is 17,750 ng/kg. In one embodiment, the dose amount is between about 18,000 ng/kg and about 22,000 ng/kg. In one embodiment, the dose amount is between about 19,000 ng/kg and about 21,000 ng/kg. In one embodiment, the dose amount is about 20,000 ng/kg. In one embodiment, the dose amount is 20,000 ng/kg.

PARAGRAPH Q includes any one of the following dose amounts: In one embodiment, the dose amount is between about 20,000 ng/kg and about 50,000 ng/kg. In one embodiment, the dose amount is between about 25,000 ng/kg and about 45,000 ng/kg. In one embodiment, the dose amount is between about 30,000 ng/kg and about 40,000 ng/kg. In one embodiment, the dose amount is between about 31,000 ng/kg and about 38,000 ng/kg. In one embodiment, the dose amount is between about 34,000 ng/kg and about 36,000 ng/kg. In one embodiment, the dose amount is about 35,000 ng/kg. In one embodiment, the dose amount is 35,000 ng/kg.

### V. a) ii). Administration Time

In one embodiment, the dose to the human subject is administered between about 5 minutes and about 10 hours. In one embodiment, the dose to the human subject is administered between about 5 minutes and about 5 hours. In one embodiment, the dose to the human subject is administered between about 5 minutes and about 60 minutes. In one embodiment, the dose to the human subject is administered between about 5 minutes and about 30 minutes. In one embodiment, the dose to the human subject is administered between about 30 minutes and about 60 minutes. In one embodiment, the dose to the human subject is administered between about 60 minutes and about 90 minutes. In one embodiment, the dose to the human subject is administered between about 90 minutes and about 2 hours. In one embodiment, the dose to the human subject is administered between about one hour and about three hours. In one embodiment, the dose to the human subject is administered between about two hours and about four hours. In one embodiment, the dose to the human subject is administered between about three hours and about five hours. In one embodiment, the dose to the human subject is administered between about four hours and about six hours. In one embodiment, the dose to the human subject is administered between about five hours and about seven hours. In one embodiment, the dose to the human subject is administered between about six hours and about eight hours. In one embodiment, the dose to the human subject is administered between about seven hours and about nine hours. In one embodiment, the dose to the human subject is administered between about eight hours and about ten hours. In one embodiment, the dose to the human subject is administered over about one hour or about three hours or about four hours or about five hours or about six hours or about seven hours or about eight hours or about nine hours or about ten hours. In one embodiment, the dose to the human subject is administered between about 90 minutes and about 150 minutes. In one embodiment, the dose to the human subject is administered between about 105 minutes and about 135 minutes. In one embodiment, the dose to the human subject is administered over about two hours. In one embodiment, the dose to the human subject is administered over two hours.

### V. b). Dosage Regimen

In one embodiment, each dosage regimen comprises at least one dose of the bispecific anti-CD123×anti-CD3 antibody (e.g., XmAb14045) provided to the human subject (per week or per month/over a set period of day(s) or week(s)). Dosage regimens are adjusted to provide the optimum desired response (e.g., a therapeutic response). The efficient dosages and the dosage regimens for the bispecific anti-CD123×anti-CD3 antibodies used in the present invention depend on the disease or condition to be treated.

### Daily Dosage Regimen

In one embodiment, the bispecific anti-CD123×anti-CD3 antibody (e.g., XmAb14045) dose is provided once a day, in a dose amount disclosed in any one of Paragraph A or Paragraph B or Paragraph C or Paragraph D or Paragraph E or Paragraph F or Paragraph G or Paragraph H or Paragraph I or Paragraph J or Paragraph K or Paragraph L or Paragraph M or Paragraph N or Paragraph O or Paragraph P or Paragraph Q, or any combination thereof. The administration time can be any described throughout the specification.

### Every Other Day Dosage Regimen

In one embodiment, the bispecific anti-CD123×anti-CD3 antibody (e.g., XmAb14045) dose is provided once every other day, in a dose amount disclosed in any one of Paragraph A or Paragraph B or Paragraph C or Paragraph D or Paragraph E or Paragraph F or Paragraph G or Paragraph H or Paragraph I or Paragraph J or Paragraph K or Paragraph L or Paragraph M or Paragraph N or Paragraph O or Paragraph P or Paragraph Q, or any combination thereof. The administration time can be any described throughout the specification.

### Six Times a Week Dosage Regimen

In one embodiment, the bispecific anti-CD123×anti-CD3 antibody (e.g., XmAb14045) dose is provided six times a week, in a dose amount disclosed in any one of Paragraph A or Paragraph B or Paragraph C or Paragraph D or Paragraph E or Paragraph F or Paragraph G or Paragraph H or Paragraph I or Paragraph J or Paragraph K or Paragraph L or Paragraph M or Paragraph N or Paragraph O or Paragraph P or Paragraph Q, or any combination thereof. The administration time can be any described throughout the specification.

### Five Times a Week Dosage Regimen

In one embodiment, the bispecific anti-CD123×anti-CD3 antibody (e.g., XmAb14045) dose is provided five times a week, in a dose amount disclosed in any one of Paragraph A or Paragraph B or Paragraph C or Paragraph D or Paragraph E or Paragraph F or Paragraph G or Paragraph H or Paragraph I or Paragraph J or Paragraph K or Paragraph L or Paragraph M or Paragraph N or Paragraph O or Paragraph P or Paragraph Q, or any combination thereof. The administration time can be any described throughout the specification.

### Four Times a Week Dosage Regimen

In one embodiment, the bispecific anti-CD123×anti-CD3 antibody (e.g., XmAb14045) dose is provided four times a week, in a dose amount disclosed in any one of Paragraph A or Paragraph B or Paragraph C or Paragraph D or Paragraph E or Paragraph F or Paragraph G or Paragraph H or Paragraph I or Paragraph J or Paragraph K or Paragraph L or Paragraph M or Paragraph N or Paragraph O or Paragraph P or Paragraph Q, or any combination thereof. The administration time can be any described throughout the specification.

### Three Times a Week Dosage Regimen

In one embodiment, the bispecific anti-CD123×anti-CD3 antibody (e.g., XmAb14045) dose is provided three times a week, in a dose amount disclosed in any one of Paragraph A or Paragraph B or Paragraph C or Paragraph D or Paragraph E or Paragraph F or Paragraph G or Paragraph H or Paragraph I or Paragraph J or Paragraph K or Paragraph L or Paragraph M or Paragraph N or Paragraph O or Paragraph P or Paragraph Q, or any combination thereof. In one embodiment, the bispecific anti-CD123×anti-CD3 antibody (e.g., XmAb14045) dose is provided three times a week, where the first dose amount is disclosed in Paragraph J, and the subsequent two dose amounts are disclosed in any one of Paragraph K or Paragraph L or Paragraph M or Paragraph N or Paragraph O or Paragraph P or Paragraph Q, or any combination thereof. In one embodiment, the bispecific anti-CD123×anti-CD3 antibody (e.g., XmAb14045) dose is provided three times a week, where the first dose amount is disclosed in Paragraph J, and the subsequent two dose amounts are disclosed in Paragraph K. In one embodiment, the bispecific anti-CD123×anti-CD3 antibody (e.g., XmAb14045) dose is provided three times a week, where the first dose amount is disclosed in Paragraph J, and the subsequent two dose amounts are disclosed in Paragraph L. In one embodiment, the bispecific anti-CD123×anti-CD3 antibody (e.g., XmAb14045) dose is provided three times a week, where the first dose amount is disclosed in Paragraph J, and the subsequent two dose amounts are disclosed in Paragraph M. In one embodiment, the bispecific anti-CD123×anti-CD3 antibody (e.g., XmAb14045) dose is provided three times a week, where the first dose amount is disclosed in Paragraph J, and the subsequent two dose amounts are disclosed in Paragraph N. In one embodiment, the bispecific anti-CD123×anti-CD3 antibody (e.g., XmAb14045) dose is provided three times a week, where the first dose amount is disclosed in Paragraph J, and the subsequent two dose amounts are disclosed in Paragraph O. In one embodiment, the bispecific anti-CD123×anti-CD3 antibody (e.g., XmAb14045) dose is provided three times a week, where the first dose amount is disclosed in Paragraph J, and the subsequent two dose amounts are disclosed in Paragraph P. In one embodiment, the bispecific anti-CD123×anti-CD3 antibody (e.g., XmAb14045) dose is provided three times a week, where the first dose amount is disclosed in Paragraph J, and the subsequent two dose amounts are disclosed in Paragraph Q. The administration time can be any described throughout the specification.

### Two Times a Week Dosage Regimen

In one embodiment, the bispecific anti-CD123×anti-CD3 antibody (e.g., XmAb14045) dose is provided two times a week, in a dose amount disclosed in any one of Paragraph A or Paragraph B or Paragraph C or Paragraph D or Paragraph E or Paragraph F or Paragraph G or Paragraph H or Paragraph I or Paragraph J or Paragraph K or Paragraph L or Paragraph M or Paragraph N or Paragraph O or Paragraph P or Paragraph Q, or any combination thereof. The administration time can be any described throughout the specification.

### Once a Week Dosage Regimen

In one embodiment, the bispecific anti-CD123×anti-CD3 antibody (e.g., XmAb14045) dose is provided once a week, in a dose amount disclosed in any one of Paragraph A or Paragraph B or Paragraph C or Paragraph D or Paragraph E or Paragraph F or Paragraph G or Paragraph H or Paragraph I or Paragraph J or Paragraph K or Paragraph L or Paragraph M or Paragraph N or Paragraph O or Paragraph P or Paragraph Q, or any combination thereof. The administration time can be any described throughout the specification.

In an exemplary embodiment, the dose is administered once between about 5 and about 10 days. In an exemplary embodiment, the dose is administered once every 5-10 days. In an exemplary embodiment, the dose is administered once between about 5 and about 9 days. In an exemplary embodiment, the dose is administered once every 5-9 days. In an exemplary embodiment, the dose is administered once between about 6 and about 8 days. In an exemplary embodiment, the dose is administered once every 6-8 days. In an exemplary embodiment, the dose is administered once between about 6 and about 10 days. In an exemplary embodiment, the dose is administered once every 6-10 days. In an exemplary embodiment, the dose is administered once between about 7 and about 9 days. In an exemplary embodiment, the dose is administered once every 7-9 days. In an exemplary embodiment, the intravenous dose of XmAb14045 is administered once about every 7 days. In an exemplary embodiment, the dose is administered once every 7 days. In an exemplary embodiment, the dose is administered about once a week. In an exemplary embodiment, the intravenous dose of XmAb14045 is administered once a week.

### Every Two Weeks Dosage Regimen

In one embodiment, the bispecific anti-CD123×anti-CD3 antibody (e.g., XmAb14045) dose is provided once every two weeks, in a dose amount disclosed in any one of Paragraph A or Paragraph B or Paragraph C or Paragraph D or Paragraph E or Paragraph F or Paragraph G or Paragraph H or Paragraph I or Paragraph J or Paragraph K or Paragraph L or Paragraph M or Paragraph N or Paragraph O or Paragraph P or Paragraph Q, or any combination thereof. The administration time can be any described throughout the specification.

### Every Three Weeks Dosage Regimen

In one embodiment, the bispecific anti-CD123×anti-CD3 antibody (e.g., XmAb14045) dose is provided once every three weeks, in a dose amount disclosed in any one of Paragraph A or Paragraph B or Paragraph C or Paragraph D or Paragraph E or Paragraph F or Paragraph G or Paragraph H or Paragraph I or Paragraph J or Paragraph K or Paragraph L or Paragraph M or Paragraph N or Paragraph O or Paragraph P or Paragraph Q, or any combination thereof. The administration time can be any described throughout the specification.

### Every Four Weeks Dosage Regimen

In one embodiment, the bispecific anti-CD123×anti-CD3 antibody (e.g., XmAb14045) dose is provided once every four weeks, in a dose amount disclosed in any one of Paragraph A or Paragraph B or Paragraph C or Paragraph D or Paragraph E or Paragraph F or Paragraph G or Paragraph H or Paragraph I or Paragraph J or Paragraph K or Paragraph L or Paragraph M or Paragraph N or Paragraph O or Paragraph P or Paragraph Q, or any combination thereof. The administration time can be any described throughout the specification.

### Two Times a Month Dosage Regimen

In one embodiment, the bispecific anti-CD123×anti-CD3 antibody (e.g., XmAb14045) dose is provided two times a month, in a dose amount selected from the from the group consisting of Paragraph A or Paragraph B or Paragraph C or Paragraph D or Paragraph E or Paragraph F or Paragraph G or Paragraph H or Paragraph I or Paragraph J or Paragraph K or Paragraph L or Paragraph M or Paragraph N or Paragraph O or Paragraph P or Paragraph Q, or any combination thereof. The administration time can be any described throughout the specification.

### Three Times a Month Dosage Regimen

In one embodiment, the bispecific anti-CD123×anti-CD3 antibody (e.g., XmAb14045) dose is provided three times a month, in a dose amount disclosed in any one of Paragraph A or Paragraph B or Paragraph C or Paragraph D or Paragraph E or Paragraph F or Paragraph G or Paragraph H or Paragraph I or Paragraph J or Paragraph K or Paragraph L or Paragraph M or Paragraph N or Paragraph O or Paragraph P or Paragraph Q, or any combination thereof. The administration time can be any described throughout the specification.

### Monthly Dosage Regimen

In one embodiment, the bispecific anti-CD123×anti-CD3 antibody (e.g., XmAb14045) dose is provided once a month, in a dose amount disclosed in any one of Paragraph A or Paragraph B or Paragraph C or Paragraph D or Paragraph E or Paragraph F or Paragraph G or Paragraph H or Paragraph I or Paragraph J or Paragraph K or Paragraph L or Paragraph M or Paragraph N or Paragraph O or Paragraph P or Paragraph Q, or any combination thereof. The administration time can be any described throughout the specification.

### V. c). Phase

In one embodiment, a phase comprises a certain number of occurrences of a dosage regimen. In one embodiment, a dosage regimen occurs one time in a phase. In one embodiment, a dosage regimen occurs two times in a phase. In one embodiment, a dosage regimen occurs three times in a phase. In one embodiment, a dosage regimen occurs four times in a phase. In one embodiment, a dosage regimen occurs five times in a phase. In one embodiment, a dosage regimen occurs six times in a phase. In one embodiment, a dosage regimen occurs seven times in a phase. In one embodiment, a dosage regimen occurs eight times in a phase. In one embodiment, a dosage regimen occurs nine times in a phase. In one embodiment, a dosage regimen occurs ten times in a phase. In one embodiment, a dosage regimen occurs eleven times in a phase. In one embodiment, a dosage regimen occurs twelve times in a phase. In one embodiment, a dosage regimen occurs thirteen times in a phase. In one embodiment, a dosage regimen occurs fourteen times in a phase. In one embodiment, a dosage regimen occurs fifteen times in a phase. In one embodiment, a dosage regimen occurs sixteen times in a phase. In one embodiment, a dosage regimen occurs seventeen times in a phase. In one embodiment, a dosage regimen occurs eighteen times in a phase. In one embodiment, a dosage regimen occurs nineteen times in a phase. In one embodiment, a dosage regimen occurs twenty times in a phase. In one embodiment, a dosage regimen continues until the cancer (e.g., hematological cancer) is in remission (e.g., complete or partial).

In one embodiment, the phase is a once a week dosage regimen described herein, with a duration of one week. In one embodiment, the phase is a once a week dosage regimen described herein, with a duration of two weeks. In one embodiment, the phase is a once a week dosage regimen described herein, with a duration of three weeks. In one embodiment, the phase is a once a week dosage regimen described herein, with a duration of four weeks.

In one embodiment, the phase is a two times a week dosage regimen described herein, with a duration of one week. In one embodiment, the phase is a two times a week dosage regimen described herein, with a duration of two weeks. In one embodiment, the phase is a two times a week dosage regimen described herein, with a duration of three weeks. In one embodiment, the phase is a two times a week dosage regimen described herein, with a duration of four weeks.

In one embodiment, the phase is a two times a week dosage regimen, where the first dose amount is different from the second dose amount. In one embodiment, the phase is a three times a week dosage regimen, where the first dose amount is smaller than the second dose amount. In one embodiment, the phase is a three times a week dosage regimen, where the first dose amount is about 750 ng/kg, and the second dose amount is in Paragraphs K or L or M or N or O or P or Q.

In one embodiment, the phase is a three times a week dosage regimen described herein, with a duration of one week. In one embodiment, the phase is a three times a week dosage regimen described herein, with a duration of two weeks. In one embodiment, the phase is a three times a week dosage regimen described herein, with a duration of three weeks. In one embodiment, the phase is a three times a week dosage regimen described herein, with a duration of four weeks.

In one embodiment, the phase is a three times a week dosage regimen, where the first dose amount is different from the subsequent two dose amounts. In one embodiment, the phase is a three times a week dosage regimen, where the first dose amount is smaller from the subsequent two dose amounts. In one embodiment, the phase is a three times a week dosage regimen, where the first dose amount is about 750 ng/kg, and the subsequent two dose amounts are each independently selected from Paragraphs K, L, M, N, O, P, and Q.

In one embodiment, the phase is a four times a week dosage regimen described herein, with a duration of one week. In one embodiment, the phase is a four times a week dosage regimen described herein, with a duration of two weeks. In one embodiment, the phase is a four times a week dosage regimen described herein, with a duration of three weeks. In one embodiment, the phase is a four times a week dosage regimen described herein, with a duration of four weeks.

In one embodiment, the phase is a four times a week dosage regimen, where the first dose amount is different from the subsequent three dose amounts. In one embodiment, the phase is a four times a week dosage regimen, where the first dose amount is smaller from the subsequent two dose amounts. In one embodiment, the phase is a four times a week dosage regimen, where the first dose amount is about 750 ng/kg, and the subsequent three dose amounts are each independently selected from Paragraphs K, L, M, N, O, P, and Q.

In one embodiment, the phase is a five times a week dosage regimen described herein, with a duration of one week. In one embodiment, the phase is a five times a week dosage regimen described herein, with a duration of two weeks. In one embodiment, the phase is a five times a week dosage regimen described herein, with a duration of three weeks. In one embodiment, the phase is a five times a week dosage regimen described herein, with a duration of four weeks.

In one embodiment, the phase is a five times a week dosage regimen, where the first dose amount is different from the subsequent four dose amounts. In one embodiment, the phase is a five times a week dosage regimen, where the first dose amount is smaller from the subsequent four dose amounts. In one embodiment, the phase is a five times a week dosage regimen, where the first dose amount is about 750 ng/kg, and the subsequent four dose amounts are each independently selected from Paragraphs K, L, M, N, O, P, and Q.

### VI. Embodiments

The method of treatment disclosed herein can comprise a first phase, for example, where the first phase is administered according to a specific dosage regimen, a specific dose amount, and for a specific administration time. In one embodiment, the method comprises a first phase where the bispecific antibody is provided daily. In one embodiment, the method comprises a first phase where the bispecific antibody is provided every other day. In one embodiment, the method comprises a first phase where the bispecific antibody is provided six times a week. In one embodiment, the method comprises a first phase where the bispecific antibody is provided five times a week. In one embodiment, the method comprises a first phase where the bispecific antibody is provided four times a week. In one embodiment, the method comprises a first phase where the bispecific antibody is provided three times a week. In one embodiment, the method comprises a first phase where the bispecific antibody is provided two times a week. In one embodiment, the method comprises a first phase where the bispecific antibody is provided once a week.

In some embodiments, where the method comprises a first phase where the bispecific antibody is provided daily, the dose amount can be any one of the dose amounts as described in Paragraph C or Paragraph D or Paragraph E or Paragraph F or Paragraph G or Paragraph H or Paragraph I or Paragraph J or Paragraph K. This first phase can continue until the cancer (e.g., a CD123-expressing cancer) is in remission.

In some embodiments, where the method comprises a first phase where the bispecific antibody is provided every other day, the dose amount can be any one of the dose amounts as described in Paragraph C or Paragraph D or Paragraph E or Paragraph F or Paragraph G or Paragraph H or Paragraph I or Paragraph J or Paragraph K. This first phase can continue until the cancer (e.g., a CD123-expressing cancer) is in remission.

In some embodiments, where the method comprises a first phase where the bispecific antibody is provided six times a week, the dose amount can be any one of the dose amounts as described in Paragraph C or Paragraph D or Paragraph E or Paragraph F or Paragraph G or Paragraph H or Paragraph I or Paragraph J or Paragraph K. This first phase can continue until the cancer (e.g., a CD123-expressing cancer) is in remission.

In some embodiments, where the method comprises a first phase where the bispecific antibody is provided five times a week, the dose amount can be any one of the dose amounts as described in Paragraph C or Paragraph D or Paragraph E or Paragraph F or Paragraph G or Paragraph H or Paragraph I or Paragraph J or Paragraph K. This first phase can continue until the cancer (e.g., a CD123-expressing cancer) is in remission.

In one embodiment, where the method comprises a first phase where the bispecific antibody is provided four times a week, the dose amount can be any one of the dose amounts as described in Paragraph C or Paragraph D or Paragraph E or Paragraph F or Paragraph G or Paragraph H or Paragraph I or Paragraph J or Paragraph K. This first phase can continue until the cancer (e.g., a CD123-expressing cancer) is in remission.

In one embodiment, where the method comprises a first phase where the bispecific antibody is provided three times a week, the dose amount can be any one of the dose amounts as described in Paragraph E or Paragraph F or Paragraph G or Paragraph H or Paragraph I or Paragraph J or Paragraph K. This first phase can continue until the cancer (e.g., a CD123-expressing cancer) is in remission.

In one embodiment, where the method comprises a first phase where the bispecific antibody is provided two times a week, the dose amount can be any one of the dose amounts as described in Paragraph G or Paragraph H or Paragraph I or Paragraph J or Paragraph K or Paragraph L. This first phase can continue until the cancer (e.g., a CD123-expressing cancer) is in remission.

In one embodiment, where the method comprises a first phase where the bispecific antibody is provided once a week, and the dose amount can be any one of the dose amounts described in Paragraph I, and for any administration time described herein. This first phase can continue until the cancer (e.g., a CD123-expressing cancer) is in remission. For any of the embodiments in this paragraph, the dose amount is about 500 ng/kg.

In one embodiment, where the method comprises a first phase where the bispecific antibody is provided once a week, and the dose amount can be any one of the dose amounts described in Paragraph J, and for any administration time described herein. This first phase can continue until the cancer (e.g., a CD123-expressing cancer) is in remission. For any of the embodiments in this paragraph, the dose amount is about 750 ng/kg.

In one embodiment, where the method comprises a first phase where the bispecific antibody is provided once a week, and the dose amount can be any one of the dose amounts described in Paragraph K, and for any administration time described herein. This first phase can continue until the cancer (e.g., a CD123-expressing cancer) is in remission. For any of the embodiments in this paragraph, the dose amount is about 1,300 ng/kg.

In one embodiment, where the method comprises a first phase where the bispecific antibody is provided once a week, and the dose amount can be any one of the dose amounts described in Paragraph L, and for any administration time described herein. This first phase can continue until the cancer (e.g., a CD123-expressing cancer) is in remission. For any of the embodiments in this paragraph, the dose amount is about 2,300 ng/kg.

In one embodiment, where the method comprises a first phase where the bispecific antibody is provided once a week, and the dose amount can be any one of the dose amounts described in Paragraph M, and for any administration time described herein. This first phase can continue until the cancer (e.g., a CD123-expressing cancer) is in remission. For any of the embodiments in this paragraph, the dose amount is about 4,000 ng/kg.

In one embodiment, where the method comprises a first phase where the bispecific antibody is provided once a week, and the dose amount can be any one of the dose amounts described in Paragraph N, and for any administration time described herein. This first phase can continue until the cancer (e.g., a CD123-expressing cancer) is in remission. For any of the embodiments in this paragraph, the dose amount is about 7,000 ng/kg.

In one embodiment, where the method comprises a first phase where the bispecific antibody is provided once a week, and the dose amount can be any one of the dose amounts described in Paragraph O, and for any administration time described herein. This first phase can continue until the cancer (e.g., a CD123-expressing cancer) is in remission. For any of the embodiments in this paragraph, the dose amount is about 12,000 ng/kg.

In one embodiment, where the method comprises a first phase where the bispecific antibody is provided once a week, and the dose amount can be any one of the dose amounts described in Paragraph P, and for any administration time described herein. This first phase can continue until the cancer (e.g., a CD123-expressing cancer) is in remission. For any of the embodiments in this paragraph, the dose amount is about 20,000 ng/kg.

In one embodiment, where the method comprises a first phase where the bispecific antibody is provided once a week, and the dose amount can be any one of the dose amounts described in Paragraph Q, and for any administration time described herein. This first phase can continue until the cancer (e.g., a CD123-expressing cancer) is in remission. For any of the embodiments in this paragraph, the dose amount is about 35,000 ng/kg.

### Two Phases

The method of treatment disclosed herein can comprise a first phase and a second phase. In the first phase, the antibody can be provided according to a first dosage regimen, with a first dose amount. In the second phase, the antibody can be provided according to a second dosage regimen, with a second dose amount. The administration times can independently be any described throughout the specification.

The method of treatment disclosed herein can comprise a first phase and a second phase. In the first phase, the antibody can be provided according to a first dosage regimen, with a first dose amount, where the first dose amount is described within Paragraph I or Paragraph J. In the second phase, the antibody can be provided according to a second dosage regimen, with a second dose amount.

The method of treatment disclosed herein can comprise a first phase and a second phase. In the first phase, the antibody can be provided according to a first dosage regimen, with a first dose amount, where the first dose amount is between about 100 ng/kg and about 750 ng/kg. In the second phase, the antibody can be provided according to a second dosage regimen, with a second dose amount.

The method of treatment disclosed herein can comprise a first phase and a second phase. In the first phase, the antibody can be provided according to a first dosage regimen, with a first dose amount, where the first dose amount is between about 600 ng/kg and about 750 ng/kg. In the second phase, the antibody can be provided according to a second dosage regimen, with a second dose amount. The administration times can independently be any described throughout the specification.

The method of treatment disclosed herein can comprise a first phase and a second phase, where during the first phase the antibody is provided once a week at a first dose amount, and where during the second phase the antibody is provided once a week in a second dose amount. In one embodiment, the first and second dose amounts are not the same. The administration times can independently be any described throughout the specification.

The method of treatment disclosed herein can comprise a first phase and a second phase, where during the first phase the antibody is provided once a week at a first dose amount described within Paragraph I or Paragraph J, and where during the second phase the antibody is provided once a week in a second dose amount. The method of treatment disclosed herein can comprise a first phase and a second phase, where during the first phase the antibody is provided once a week at a first dose amount which is between about 100 ng/kg and about 750 ng/kg, and where during the second phase the antibody is provided once a week in a second dose amount. The method of treatment disclosed herein can comprise a first phase and a second phase, where during the first phase the antibody is provided once a week at a first dose amount which is between about 600 ng/kg and about 750 ng/kg, and where during the second phase the antibody is provided once a week in a second dose amount. In one embodiment, the first and second dose amounts are not the same. The administration times can independently be any described throughout the specification.

In one embodiment, the method of treatment comprises a first phase and a second phase, where during the first phase, the antibody is administered in a first dose amount described within any one of Paragraph I or Paragraph J or Paragraph K or Paragraph L or Paragraph M or Paragraph N or Paragraph O or Paragraph P or Paragraph Q, and where during the second phase the antibody is administered according to a second dose amount described within any one of Paragraph I or Paragraph J or Paragraph K or Paragraph L or Paragraph M or Paragraph N or Paragraph O or Paragraph P or Paragraph Q. In one embodiment, the first and second dose amounts are not the same. The administration times can independently be any described throughout the specification.

In one embodiment, the method of treatment comprises a first phase and a second phase, where during the first phase, the antibody is administered in a first dose amount described within Paragraph I or Paragraph J, and where during the second phase the antibody is administered according to a second dose amount described within any one of Paragraph K or Paragraph L or Paragraph M or Paragraph N or Paragraph O or Paragraph P or Paragraph Q. In one embodiment, the first and second dose amounts are not the same. In one embodiment, the method of treatment comprises a first phase and a second phase, where during the first phase, the antibody is administered in a first dose amount between about 100 ng/kg and about 750 ng/kg, and where during the second phase the antibody is administered according to a second dose amount described within any one of Paragraph K or Paragraph L or Paragraph M or Paragraph N or Paragraph O or Paragraph P or Paragraph Q. In one embodiment, the first and second dose amounts are not the same. In one embodiment, the method of treatment comprises a first phase and a second phase, where during the first phase, the antibody is administered in a first dose amount between about 600 ng/kg and about 750 ng/kg, and where during the second phase the antibody is administered according to a second dose amount described within any one of Paragraph K or Paragraph L or Paragraph M or Paragraph N or Paragraph O or Paragraph P or Paragraph Q. In one embodiment, the first and second dose amounts are not the same. The administration times can independently be any described throughout the specification.

In one embodiment, the method of treatment comprises a first phase and a second phase, where during the first phase, the antibody is provided once a week in a first dose amount described within any one of Paragraph I or Paragraph J or Paragraph K or Paragraph L or Paragraph M or Paragraph N or Paragraph O or Paragraph P or Paragraph Q, and where during the second phase the antibody is provided once a week according to a second dose amount described within any one of Paragraph I or Paragraph J or Paragraph K or Paragraph L or Paragraph M or Paragraph N or Paragraph O or Paragraph P or Paragraph Q. In one embodiment, the first and second dose amounts are not the same. The administration times can independently be any described throughout the specification.

In one embodiment, the method of treatment comprises a first phase and a second phase, where during the first phase, the antibody is provided once a week in a first dose amount described within Paragraph I or Paragraph J, and where during the second phase the antibody is provided once a week according to a second dose amount described within any one of Paragraph I or Paragraph J or Paragraph K or Paragraph L or Paragraph M or Paragraph N or Paragraph O or Paragraph P or Paragraph Q. In one embodiment, the method of treatment comprises a first phase and a second phase, where during the first phase, the antibody is provided once a week in a first dose amount between about 100 ng/kg and about 750 ng/kg, and where during the second phase the antibody is provided once a week according to a second dose amount described within any one of Paragraph K or Paragraph L or Paragraph M or Paragraph N or Paragraph O or Paragraph P or Paragraph Q. In one embodiment, the method of treatment comprises a first phase and a second phase, where during the first phase, the antibody is provided once a week in a first dose amount between about 600 ng/kg and about 750 ng/kg, and where during the second phase the antibody is provided once a week according to a second dose amount described within any one of Paragraph K or Paragraph L or Paragraph M or Paragraph N or Paragraph O or Paragraph P or Paragraph Q. In one embodiment, the first and second dose amounts are not the same. The administration times can independently be any described throughout the specification.

In one embodiment, the method of treatment comprises a first phase and a second phase, where during the first phase, the antibody is provided once a week for up to four weeks (e.g., one, two, three, or four weeks), in a first dose amount described within any one of Paragraph I or Paragraph J or Paragraph K or Paragraph L or Paragraph M or Paragraph N or Paragraph O or Paragraph P or Paragraph Q, and where during the second phase the antibody is provided once a week until remission, according to a second dose amount described within any one of Paragraph I or Paragraph J or Paragraph K or Paragraph L or Paragraph M or Paragraph N or Paragraph O or Paragraph P or Paragraph Q. In one embodiment, the first and second dose amounts are not the same. The administration times can independently be any described throughout the specification.

In one embodiment, the method of treatment comprises a first phase and a second phase, where during the first phase, the antibody is provided once a week for up to four weeks (e.g., one, two, three, or four weeks), in a first dose amount described within Paragraph I or Paragraph J, and where during the second phase the antibody is provided once a week until remission, according to a second dose amount described within any one of Paragraph I or Paragraph J or Paragraph K or Paragraph L or Paragraph M or Paragraph N or Paragraph O or Paragraph P or Paragraph Q. In one embodiment, the method of treatment comprises a first phase and a second phase, where during the first phase, the antibody is provided once a week for up to four weeks (e.g., one, two, three, or four weeks), in a first dose amount between about 100 ng/kg and about 750 ng/kg, and where during the second phase the antibody is provided once a week until remission, according to a second dose amount described within any one of Paragraph K or Paragraph L or Paragraph M or Paragraph N or Paragraph O or Paragraph P or Paragraph Q. In one embodiment, the method of treatment comprises a first phase and a second phase, where during the first phase, the antibody is provided once a week for up to four weeks (e.g., one, two, three, or four weeks), in a first dose amount between about 600 ng/kg and about 750 ng/kg, and where during the second phase the antibody is provided once a week until remission, according to a second dose amount described within any one of Paragraph K or Paragraph L or Paragraph M or Paragraph N or Paragraph O or Paragraph P or Paragraph Q. In one embodiment, the first and second dose amounts are not the same. The administration times can independently be any described throughout the specification.

### Three Phases

In one embodiment, the method of treatment comprises a first phase, a second phase, and a third phase, where during the first phase, the antibody is administered in a first dose amount, where during the second phase, the antibody is administered in a second dose amount, and where during the third phase, the antibody is administered in a third dose amount. In one embodiment, the first, second, and third dose amounts are not the same. In one embodiment, the first, second, and third dose amounts are not the same (i.e., two are the same and one is different). The administration times can independently be any described throughout the specification.

In one embodiment, the method of treatment comprises a first phase, a second phase, and a third phase, where during the first phase, the antibody is administered in a first dose amount described within Paragraph I or Paragraph J, where during the second phase, the antibody is administered in a second dose amount, and where during the third phase, the antibody is administered in a third dose amount. In one embodiment, the method of treatment comprises a first phase, a second phase, and a third phase, where during the first phase, the antibody is administered in a first dose amount which is between about 100 ng/kg and about 750 ng/kg, where during the second phase, the antibody is administered in a second dose amount, and where during the third phase, the antibody is administered in a third dose amount. In one embodiment, the method of treatment comprises a first phase, a second phase, and a third phase, where during the first phase, the antibody is administered in a first dose amount which is between about 600 ng/kg and about 750 ng/kg, where during the second phase, the antibody is administered in a second dose amount, and where during the third phase, the antibody is administered in a third dose amount. In one embodiment, the first, second, and third dose amounts are not the same. In one embodiment, the first, second, and third dose amounts are not the same (i.e., two are the same and one is different). The administration times can independently be any described throughout the specification.

In one embodiment, the method of treatment comprises a first phase, a second phase, and a third phase, where during the first phase the antibody is provided once a week in a first dose amount, where during the second phase, the antibody is provided once a week in a second dose amount, and where during the third phase, the antibody is provided once a week in a third dose amount. In one embodiment, the first, second, and third dose amounts are not the same. In one embodiment, the first, second, and third dose amounts are not the same (i.e., two are the same and one is different). The administration times can independently be any described throughout the specification.

In one embodiment, the method of treatment comprises a first phase, a second phase, and a third phase, where during the first phase the antibody is provided once a week in a first dose amount which is described within Paragraph I or Paragraph J, where during the second phase, the antibody is provided once a week in a second dose amount, and where during the third phase, the antibody is provided once a week in a third dose amount. In one embodiment, the method of treatment comprises a first phase, a second phase, and a third phase, where during the first phase the antibody is provided once a week in a first dose amount which is between about 100 ng/kg and about 750 ng/kg, where during the second phase, the antibody is provided once a week in a second dose amount, and where during the third phase, the antibody is provided once a week in a third dose amount. In one embodiment, the method of treatment comprises a first phase, a second phase, and a third phase, where during the first phase the antibody is provided once a week in a first dose amount which is between about 600 ng/kg and about 750 ng/kg, where during the second phase, the antibody is provided once a week in a second dose amount, and where during the third phase, the antibody is provided once a week in a third dose amount. In one embodiment, the first, second, and third dose amounts are not the same. In one embodiment, the first, second, and third dose amounts are not the same (i.e., two are the same and one is different). The administration times can independently be any described throughout the specification.

In one embodiment, the method of treatment comprises a first phase, a second phase, and a third phase, where during the first phase the antibody is administered in a first dose amount described within any one of Paragraph I or Paragraph J or Paragraph K or Paragraph L or Paragraph M or Paragraph N or Paragraph O or Paragraph P or Paragraph Q, where during the second phase the antibody is administered in a second dose amount described within any one of Paragraph I or Paragraph J or Paragraph K or Paragraph L or Paragraph M or Paragraph N or Paragraph O or Paragraph P or Paragraph Q, and where during the third phase the antibody is administered in a third dose amount described within any one of Paragraph I or Paragraph J or Paragraph K or Paragraph L or Paragraph M or Paragraph N or Paragraph O or Paragraph P or Paragraph Q. In one embodiment, the first, second, and third dose amounts are not the same. In one embodiment, the first, second, and third dose amounts are not the same (i.e., two are the same and one is different). The administration times can independently be any described throughout the specification.

In one embodiment, the method of treatment comprises a first phase, a second phase, and a third phase, where during the first phase the antibody is administered in a first dose amount which is described within Paragraph I or Paragraph J, where during the second phase the antibody is administered in a second dose amount described within any one of Paragraph I or Paragraph J or Paragraph K or Paragraph L or Paragraph M or Paragraph N or Paragraph O or Paragraph P or Paragraph Q, and where during the third phase the antibody is administered in a third dose amount described within any one of Paragraph I or Paragraph J or Paragraph K or Paragraph L or Paragraph M or Paragraph N or Paragraph O or Paragraph P or Paragraph Q. In one embodiment, the method of treatment comprises a first phase, a second phase, and a third phase, where during the first phase the antibody is administered in a first dose amount which is between about 100 ng/kg and about 750 ng/kg, where during the second phase the antibody is administered in a second dose amount described within any one of Paragraph I or Paragraph J or Paragraph K or Paragraph L or Paragraph M or Paragraph N or Paragraph O or Paragraph P or Paragraph Q, and where during the third phase the antibody is administered in a third dose amount described within any one of Paragraph I or Paragraph J or Paragraph K or Paragraph L or Paragraph M or Paragraph N or Paragraph O or Paragraph P or Paragraph Q. In one embodiment, the method of treatment comprises a first phase, a second phase, and a third phase, where during the first phase the antibody is administered in a first dose amount which is between about 600 ng/kg and about 750 ng/kg, where during the second phase the antibody is administered in a second dose amount described within any one of Paragraph I or Paragraph J or Paragraph K or Paragraph L or Paragraph M or Paragraph N or Paragraph O or Paragraph P or Paragraph Q, and where during the third phase the antibody is administered in a third dose amount described within any one of Paragraph I or Paragraph J or Paragraph K or Paragraph L or Paragraph M or Paragraph N or Paragraph O or Paragraph P or Paragraph Q. In one embodiment, the first, second, and third dose amounts are not the same. In one embodiment, the first, second, and third dose amounts are not the same (i.e., two are the same and one is different). The administration times can independently be any described throughout the specification.

In one embodiment, the method of treatment comprises a first phase, a second phase, and a third phase, where during the first phase the antibody is provided once a week in a first dose amount described within any one of Paragraph I or Paragraph J or Paragraph K or Paragraph L or Paragraph M or Paragraph N or Paragraph O or Paragraph P or Paragraph Q, or any combination thereof, where during the second phase the antibody is provided once a week in a second dose amount described within any one of Paragraph I or Paragraph J or Paragraph K or Paragraph L or Paragraph M or Paragraph N or Paragraph O or Paragraph P or Paragraph Q, or any combination thereof, and where during the third phase the antibody is provided once a week in a third dose amount described within any one of Paragraph I or Paragraph J or Paragraph K or Paragraph L or Paragraph M or Paragraph N or Paragraph O or Paragraph P or Paragraph Q, or any combination thereof. In one embodiment, the first, second, and third dose amounts are not the same. In one embodiment, the first, second, and third dose amounts are not the same (i.e., two are the same and one is different). The administration times can independently be any described throughout the specification.

In one embodiment, the method of treatment comprises a first phase, a second phase, and a third phase, where during the first phase the antibody is provided once a week in a first dose amount described within Paragraph I or Paragraph J, or any combination thereof, where during the second phase the antibody is provided once a week in a second dose amount described within any one of Paragraph I or Paragraph J or Paragraph K or Paragraph L or Paragraph M or Paragraph N or Paragraph O or Paragraph P or Paragraph Q, or any combination thereof, and where during the third phase the antibody is provided once a week in a third dose amount described within any one of Paragraph I or Paragraph J or Paragraph K or Paragraph L or Paragraph M or Paragraph N or Paragraph O or Paragraph P or Paragraph Q, or any combination thereof. In one embodiment, the method of treatment comprises a first phase, a second phase, and a third phase, where during the first phase the antibody is provided once a week in a first dose amount which is between about 100 ng/kg and about 750 ng/kg, or any combination thereof, where during the second phase the antibody is provided once a week in a second dose amount described within any one of Paragraph I or Paragraph J or Paragraph K or Paragraph L or Paragraph M or Paragraph N or Paragraph O or Paragraph P or Paragraph Q, or any combination thereof, and where during the third phase the antibody is provided once a week in a third dose amount described within any one of Paragraph I or Paragraph J or Paragraph K or Paragraph L or Paragraph M or Paragraph N or Paragraph O or Paragraph P or Paragraph Q, or any combination thereof. In one embodiment, the method of treatment comprises a first phase, a second phase, and a third phase, where during the first phase the antibody is provided once a week in a first dose amount which is between about 600 ng/kg and about 750 ng/kg, or any combination thereof, where during the second phase the antibody is provided once a week in a second dose amount described within any one of Paragraph I or Paragraph J or Paragraph K or Paragraph L or Paragraph M or Paragraph N or Paragraph O or Paragraph P or Paragraph Q, or any combination thereof, and where during the third phase the antibody is provided once a week in a third dose amount described within any one of Paragraph I or Paragraph J or Paragraph K or Paragraph L or Paragraph M or Paragraph N or Paragraph O or Paragraph P or Paragraph Q, or any combination thereof. In one embodiment, the first, second, and third dose amounts are not the same. In one embodiment, the first, second, and third dose amounts are not the same (i.e., two are the same and one is different). The administration times can independently be any described throughout the specification.

In one embodiment, the method of treatment comprises a first phase, a second phase, and a third phase, where during the first phase the antibody is provided once a week for up to four weeks (e.g., one, two, three, or four weeks), in a first dose amount described within any one of Paragraph I or Paragraph J or Paragraph K or Paragraph L or Paragraph M or Paragraph N or Paragraph O or Paragraph P or Paragraph Q, or any combination thereof, where during the second phase the antibody is provided once a week for up to four weeks (e.g., one, two, three, or four weeks), in a second dose amount described within any one of Paragraph I or Paragraph J or Paragraph K or Paragraph L or Paragraph M or Paragraph N or Paragraph O or Paragraph P or Paragraph Q, or any combination thereof, and where during the third phase the antibody is provided once a week once a week until remission, in a third dose amount described within any one of Paragraph I or Paragraph J or Paragraph K or Paragraph L or Paragraph M or Paragraph N or Paragraph O or Paragraph P or Paragraph Q, or any combination thereof. In one embodiment, the first, second, and third dose amounts are not the same. In one embodiment, the first, second, and third dose amounts are not the same (i.e., two are the same and one is different). The administration times can independently be any described throughout the specification.

In one embodiment, the method of treatment comprises a first phase, a second phase, and a third phase, where during the first phase the antibody is provided once a week for up to four weeks (e.g., one, two, three, or four weeks), in a first dose amount described within Paragraph I or Paragraph J, or any combination thereof, where during the second phase the antibody is provided once a week for up to four weeks (e.g., one, two, three, or four weeks), in a second dose amount described within any one of Paragraph I or Paragraph J or Paragraph K or Paragraph L or Paragraph M or Paragraph N or Paragraph O or Paragraph P or Paragraph Q, or any combination thereof, and where during the third phase the antibody is provided once a week once a week until remission, in a third dose amount described within any one of Paragraph I or Paragraph J or Paragraph K or Paragraph L or Paragraph M or Paragraph N or Paragraph O or Paragraph P or Paragraph Q, or any combination thereof. In one embodiment, the method of treatment comprises a first phase, a second phase, and a third phase, where during the first phase the antibody is provided once a week for up to four weeks (e.g., one, two, three, or four weeks), in a first dose amount which is between about 100 ng/kg and about 750 ng/kg, or any combination thereof, where during the second phase the antibody is provided once a week for up to four weeks (e.g., one, two, three, or four weeks), in a second dose amount described within any one of Paragraph I or Paragraph J or Paragraph K or Paragraph L or Paragraph M or Paragraph N or Paragraph O or Paragraph P or Paragraph Q, or any combination thereof, and where during the third phase the antibody is provided once a week once a week until remission, in a third dose amount described within any one of Paragraph I or Paragraph J or Paragraph K or Paragraph L or Paragraph M or Paragraph N or Paragraph O or Paragraph P or Paragraph Q, or any combination thereof.

In one embodiment, the method of treatment comprises a first phase, a second phase, and a third phase, where during the first phase the antibody is provided once a week for up to four weeks (e.g., one, two, three, or four weeks), in a first dose amount which is between about 600 ng/kg and about 750 ng/kg, or any combination thereof, where during the second phase the antibody is provided once a week for up to four weeks (e.g., one, two, three, or four weeks), in a second dose amount described within any one of Paragraph I or Paragraph J or Paragraph K or Paragraph L or Paragraph M or Paragraph N or Paragraph O or Paragraph P or Paragraph Q, or any combination thereof, and where during the third phase the antibody is provided once a week once a week until remission, in a third dose amount described within any one of Paragraph I or Paragraph J or Paragraph K or Paragraph L or Paragraph M or Paragraph N or Paragraph O or Paragraph P or Paragraph Q, or any combination thereof. In one embodiment, the first, second, and third dose amounts are not the same. In one embodiment, the first, second, and third dose amounts are not the same (i.e., two are the same and one is different). The administration times can independently be any described throughout the specification.

### Four Phases

The method of treatment disclosed herein can comprise a first phase, a second phase, a third phase, and a fourth phase, where during the first phase the antibody is administered in a first dose amount, where during the second phase the antibody is administered in a second dose amount, where during the third phase the antibody is administered in a third dose amount, and where during the fourth phase the antibody is administered in a fourth dose amount. In one embodiment, the first, second, third, and fourth amounts are the same. In one embodiment, three of the first, second, third, and fourth dose amounts not the same (i.e., three are the same and one is different). In one embodiment, two of the first, second, third, and fourth dose amounts are the same (i.e., two are the same and two are different). In one embodiment, the first, second, third, and fourth amounts are different. The administration times can independently be any described throughout the specification.

The method of treatment disclosed herein can comprise a first phase, a second phase, a third phase, and a fourth phase, where during the first phase the antibody is administered in a first dose amount which is described within Paragraph I or Paragraph J, where during the second phase the antibody is administered in a second dose amount, where during the third phase the antibody is administered in a third dose amount, and where during the fourth phase the antibody is administered in a fourth dose amount. The method of treatment disclosed herein can comprise a first phase, a second phase, a third phase, and a fourth phase, where during the first phase the antibody is administered in a first dose amount which is between about 100 ng/kg and about 750 ng/kg, where during the second phase the antibody is administered in a second dose amount, where during the third phase the antibody is administered in a third dose amount, and where during the fourth phase the antibody is administered in a fourth dose amount. The method of treatment disclosed herein can comprise a first phase, a second phase, a third phase, and a fourth phase, where during the first phase the antibody is administered in a first dose amount which is between about 600 ng/kg and about 750 ng/kg, where during the second phase the antibody is administered in a second dose amount, where during the third phase the antibody is administered in a third dose amount, and where during the fourth phase the antibody is administered in a fourth dose amount. In one embodiment, the first, second, third, and fourth amounts are the same. In one embodiment, three of the first, second, third, and fourth dose amounts not the same (i.e., three are the same and one is different). In one embodiment, two of the first, second, third, and fourth dose amounts are the same (i.e., two are the same and two are different). In one embodiment, the first, second, third, and fourth amounts are different. The administration times can independently be any described throughout the specification.

In one embodiment, the method of treatment comprises a first phase, a second phase, a third phase, and a fourth phase, where during the first phase the antibody is provided once a week in a first dose amount, where during the second phase the antibody is provided once a week in a second dose amount, where during the third phase the antibody is provided once a week in a third dose amount, and where during the fourth phase the antibody is provided once a week in a fourth dose amount. In one embodiment, the first, second, third, and fourth amounts are the same. In one embodiment, three of the first, second, third, and fourth dose amounts not the same (i.e., three are the same and one is different). In one embodiment, two of the first, second, third, and fourth dose amounts are the same (i.e., two are the same and two are different). In one embodiment, the first, second, third, and fourth amounts are different. The administration times can independently be any described throughout the specification.

In one embodiment, the method of treatment comprises a first phase, a second phase, a third phase, and a fourth phase, where during the first phase the antibody is provided once a week in a first dose amount which is described within Paragraph I or Paragraph J, where during the second phase the antibody is provided once a week in a second dose amount, where during the third phase the antibody is provided once a week in a third dose amount, and where during the fourth phase the antibody is provided once a week in a fourth dose amount. In one embodiment, the method of treatment comprises a first phase, a second phase, a third phase, and a fourth phase, where during the first phase the antibody is provided once a week in a first dose amount which is between about 100 ng/kg and about 750 ng/kg, where during the second phase the antibody is provided once a week in a second dose amount, where during the third phase the antibody is provided once a week in a third dose amount, and where during the fourth phase the antibody is provided once a week in a fourth dose amount. In one embodiment, the method of treatment comprises a first phase, a second phase, a third phase, and a fourth phase, where during the first phase the antibody is provided once a week in a first dose amount which is between about 600 ng/kg and about 750 ng/kg, where during the second phase the antibody is provided once a week in a second dose amount, where during the third phase the antibody is provided once a week in a third dose amount, and where during the fourth phase the antibody is provided once a week in a fourth dose amount. In one embodiment, the first, second, third, and fourth amounts are the same. In one embodiment, three of the first, second, third, and fourth dose amounts not the same (i.e., three are the same and one is different). In one embodiment, two of the first, second, third, and fourth dose amounts are the same (i.e., two are the same and two are different). In one embodiment, the first, second, third, and fourth amounts are different. The administration times can independently be any described throughout the specification.

In one embodiment, the method of treatment comprises a first phase, a second phase, a third phase, and a fourth phase, where during the first phase the antibody is administered in a first dose amount described within any one of Paragraph I or Paragraph J or Paragraph K or Paragraph L or Paragraph M or Paragraph N or Paragraph O or Paragraph P or Paragraph Q, where during the second phase the antibody is administered in a second dose amount described within any one of Paragraph I or Paragraph J or Paragraph K or Paragraph L or Paragraph M or Paragraph N or Paragraph O or Paragraph P or Paragraph Q, where during the third phase the antibody is administered in a third dose amount described within any one of Paragraph I or Paragraph J or Paragraph K or Paragraph L or Paragraph M or Paragraph N or Paragraph O or Paragraph P or Paragraph Q, and where during the fourth phase the antibody is administered in a fourth dose amount described within any one of Paragraph I or Paragraph J or Paragraph K or Paragraph L or Paragraph M or Paragraph N or Paragraph O or Paragraph P or Paragraph Q. In one embodiment, the first, second, third, and fourth amounts are the same. In one embodiment, three of the first, second, third, and fourth dose amounts not the same (i.e., three are the same and one is different). In one embodiment, two of the first, second, third, and fourth dose amounts are the same (i.e., two are the same and two are different). In one embodiment, the first, second, third, and fourth amounts are different. The administration times can independently be any described throughout the specification.

In one embodiment, the method of treatment comprises a first phase, a second phase, a third phase, and a fourth phase, where during the first phase the antibody is administered in a first dose amount described within Paragraph I or Paragraph J, where during the second phase the antibody is administered in a second dose amount described within any one of Paragraph I or Paragraph J or Paragraph K or Paragraph L or Paragraph M or Paragraph N or Paragraph O or Paragraph P or Paragraph Q, where during the third phase the antibody is administered in a third dose amount described within any one of Paragraph I or Paragraph J or Paragraph K or Paragraph L or Paragraph M or Paragraph N or Paragraph O or Paragraph P or Paragraph Q, and where during the fourth phase the antibody is administered in a fourth dose amount described within any one of Paragraph I or Paragraph J or Paragraph K or Paragraph L or Paragraph M or Paragraph N or Paragraph O or Paragraph P or Paragraph Q. In one embodiment, the method of treatment comprises a first phase, a second phase, a third phase, and a fourth phase, where during the first phase the antibody is administered in a first dose amount which is between about 100 ng/kg and about 750 ng/kg, where during the second phase the antibody is administered in a second dose amount described within any one of Paragraph I or Paragraph J or Paragraph K or Paragraph L or Paragraph M or Paragraph N or Paragraph O or Paragraph P or Paragraph Q, where during the third phase the antibody is administered in a third dose amount described within any one of Paragraph I or Paragraph J or Paragraph K or Paragraph L or Paragraph M or Paragraph N or Paragraph O or Paragraph P or Paragraph Q, and where during the fourth phase the antibody is administered in a fourth dose amount described within any one of Paragraph I or Paragraph J or Paragraph K or Paragraph L or Paragraph M or Paragraph N or Paragraph O or Paragraph P or Paragraph Q. In one embodiment, the method of treatment comprises a first phase, a second phase, a third phase, and a fourth phase, where during the first phase the antibody is administered in a first dose amount which is between about 600 ng/kg and about 750 ng/kg, where during the second phase the antibody is administered in a second dose amount described within any one of Paragraph I or Paragraph J or Paragraph K or Paragraph L or Paragraph M or Paragraph N or Paragraph O or Paragraph P or Paragraph Q, where during the third phase the antibody is administered in a third dose amount described within any one of Paragraph I or Paragraph J or Paragraph K or Paragraph L or Paragraph M or Paragraph N or Paragraph O or Paragraph P or Paragraph Q, and where during the fourth phase the antibody is administered in a fourth dose amount described within any one of Paragraph I or Paragraph J or Paragraph K or Paragraph L or Paragraph M or Paragraph N or Paragraph O or Paragraph P or Paragraph Q. In one embodiment, the first, second, third, and fourth amounts are the same. In one embodiment, three of the first, second, third, and fourth dose amounts not the same (i.e., three are the same and one is different). In one embodiment, two of the first, second, third, and fourth dose amounts are the same (i.e., two are the same and two are different). In one embodiment, the first, second, third, and fourth amounts are different. The administration times can independently be any described throughout the specification.

In one embodiment, the method of treatment comprises a first phase, a second phase, a third phase, and a fourth phase, where during the first phase the antibody is provided once a week in a first dose amount described within any one of Paragraph I or Paragraph J or Paragraph K or Paragraph L or Paragraph M or Paragraph N or Paragraph O or Paragraph P or Paragraph Q, where during the second phase the antibody is provided once a week in a second dose amount described within any one of Paragraph I or Paragraph J or Paragraph K or Paragraph L or Paragraph M or Paragraph N or Paragraph O or Paragraph P or Paragraph Q, where during the third phase the antibody is provided once a week in a third dose amount described within any one of Paragraph I or Paragraph J or Paragraph K or Paragraph L or Paragraph M or Paragraph N or Paragraph O or Paragraph P or Paragraph Q, and where during the fourth phase the antibody is provided once a week in a fourth dose amount described within any one of Paragraph I or Paragraph J or Paragraph K or Paragraph L or Paragraph M or Paragraph N or Paragraph O or Paragraph P or Paragraph Q. In one embodiment, the first, second, third, and fourth amounts are the same. In one embodiment, three of the first, second, third, and fourth dose amounts not the same (i.e., three are the same and one is different). In one embodiment, two of the first, second, third, and fourth dose amounts are the same (i.e., two are the same and two are different). In one embodiment, the first, second, third, and fourth amounts are different. The administration times can independently be any described throughout the specification.

In one embodiment, the method of treatment comprises a first phase, a second phase, a third phase, and a fourth phase, where during the first phase the antibody is provided once a week in a first dose amount described within Paragraph I or Paragraph J, where during the second phase the antibody is provided once a week in a second dose amount described within any one of Paragraph I or Paragraph J or Paragraph K or Paragraph L or Paragraph M or Paragraph N or Paragraph O or Paragraph P or Paragraph Q, where during the third phase the antibody is provided once a week in a third dose amount described within any one of Paragraph I or Paragraph J or Paragraph K or Paragraph L or Paragraph M or Paragraph N or Paragraph O or Paragraph P or Paragraph Q, and where during the fourth phase the antibody is provided once a week in a fourth dose amount described within any one of Paragraph I or Paragraph J or Paragraph K or Paragraph L or Paragraph M or Paragraph N or Paragraph O or Paragraph P or Paragraph Q. In one embodiment, the method of treatment comprises a first phase, a second phase, a third phase, and a fourth phase, where during the first phase the antibody is provided once a week in a first dose amount which is between about 100 ng/kg and about 750 ng/kg, where during the second phase the antibody is provided once a week in a second dose amount described within any one of Paragraph I or Paragraph J or Paragraph K or Paragraph L or Paragraph M or Paragraph N or Paragraph O or Paragraph P or Paragraph Q, where during the third phase the antibody is provided once a week in a third dose amount described within any one of Paragraph I or Paragraph J or Paragraph K or Paragraph L or Paragraph M or Paragraph N or Paragraph O or Paragraph P or Paragraph Q, and where during the fourth phase the antibody is provided once a week in a fourth dose amount described within any one of Paragraph I or Paragraph J or Paragraph K or Paragraph L or Paragraph M or Paragraph N or Paragraph O or Paragraph P or Paragraph Q. In one embodiment, the method of treatment comprises a first phase, a second phase, a third phase, and a fourth phase, where during the first phase the antibody is provided once a week in a first dose amount which is between about 600 ng/kg and about 750 ng/kg, where during the second phase the antibody is provided once a week in a second dose amount described within any one of Paragraph I or Paragraph J or Paragraph K or Paragraph L or Paragraph M or Paragraph N or Paragraph O or Paragraph P or Paragraph Q, where during the third phase the antibody is provided once a week in a third dose amount described within any one of Paragraph I or Paragraph J or Paragraph K or Paragraph L or Paragraph M or Paragraph N or Paragraph O or Paragraph P or Paragraph Q, and where during the fourth phase the antibody is provided once a week in a fourth dose amount described within any one of Paragraph I or Paragraph J or Paragraph K or Paragraph L or Paragraph M or Paragraph N or Paragraph O or Paragraph P or Paragraph Q. In one embodiment, the first, second, third, and fourth amounts are the same. In one embodiment, three of the first, second, third, and fourth dose amounts not the same (i.e., three are the same and one is different). In one embodiment, two of the first, second, third, and fourth dose amounts are the same (i.e., two are the same and two are different). In one embodiment, the first, second, third, and fourth amounts are different. The administration times can independently be any described throughout the specification.

In one embodiment, the method of treatment comprises a first phase, a second phase, a third phase, and a fourth phase, where during the first phase the antibody is provided once a week for up to four weeks (e.g., one, two, three, or four weeks), in a first dose amount described within any one of Paragraph I or Paragraph J or Paragraph K or Paragraph L or Paragraph M or Paragraph N or Paragraph O or Paragraph P or Paragraph Q, where during the second phase the antibody is provided once a week for up to four weeks (e.g., one, two, three, or four weeks), in a second dose amount described within any one of Paragraph I or Paragraph J or Paragraph K or Paragraph L or Paragraph M or Paragraph N or Paragraph O or Paragraph P or Paragraph Q, where during the third phase the antibody is provided once a week for up to four weeks (e.g., one, two, three, or four weeks), in a third dose amount described within any one of Paragraph I or Paragraph J or Paragraph K or Paragraph L or Paragraph M or Paragraph N or Paragraph O or Paragraph P or Paragraph Q, and where during the fourth phase the antibody is provided once a week until remission, in a fourth dose amount described within any one of Paragraph I or Paragraph J or Paragraph K or Paragraph L or Paragraph M or Paragraph N or Paragraph O or Paragraph P or Paragraph Q. In one embodiment, the first, second, third, and fourth amounts are the same. In one embodiment, three of the first, second, third, and fourth dose amounts not the same (i.e., three are the same and one is different). In one embodiment, two of the first, second, third, and fourth dose amounts are the same (i.e., two are the same and two are different). In one embodiment, the first, second, third, and fourth amounts are different. The administration times can independently be any described throughout the specification.

In one embodiment, the method of treatment comprises a first phase, a second phase, a third phase, and a fourth phase, where during the first phase the antibody is provided once a week for up to four weeks (e.g., one, two, three, or four weeks), in a first dose amount described within Paragraph I or Paragraph J, where during the second phase the antibody is provided once a week for up to four weeks (e.g., one, two, three, or four weeks), in a second dose amount described within any one of Paragraph I or Paragraph J or Paragraph K or Paragraph L or Paragraph M or Paragraph N or Paragraph O or Paragraph P or Paragraph Q, where during the third phase the antibody is provided once a week for up to four weeks (e.g., one, two, three, or four weeks), in a third dose amount described within any one of Paragraph I or Paragraph J or Paragraph K or Paragraph L or Paragraph M or Paragraph N or Paragraph O or Paragraph P or Paragraph Q, and where during the fourth phase the antibody is provided once a week until remission, in a fourth dose amount described within any one of Paragraph I or Paragraph J or Paragraph K or Paragraph L or Paragraph M or Paragraph N or Paragraph O or Paragraph P or Paragraph Q. In one embodiment, the method of treatment comprises a first phase, a second phase, a third phase, and a fourth phase, where during the first phase the antibody is provided once a week for up to four weeks (e.g., one, two, three, or four weeks), in a first dose amount which is between about 100 ng/kg and about 750 ng/kg, where during the second phase the antibody is provided once a week for up to four weeks (e.g., one, two, three, or four weeks), in a second dose amount described within any one of Paragraph I or Paragraph J or Paragraph K or Paragraph L or Paragraph M or Paragraph N or Paragraph O or Paragraph P or Paragraph Q, where during the third phase the antibody is provided once a week for up to four weeks (e.g., one, two, three, or four weeks), in a third dose amount described within any one of Paragraph I or Paragraph J or Paragraph K or Paragraph L or Paragraph M or Paragraph N or Paragraph O or Paragraph P or Paragraph Q, and where during the fourth phase the antibody is provided once a week until remission, in a fourth dose amount described within any one of Paragraph I or Paragraph J or Paragraph K or Paragraph L or Paragraph M or Paragraph N or Paragraph O or Paragraph P or Paragraph Q. In one embodiment, the method of treatment comprises a first phase, a second phase, a third phase, and a fourth phase, where during the first phase the antibody is provided once a week for up to four weeks (e.g., one, two, three, or four weeks), in a first dose amount which is between about 600 ng/kg and about 750 ng/kg, where during the second phase the antibody is provided once a week for up to four weeks (e.g., one, two, three, or four weeks), in a second dose amount described within any one of Paragraph I or Paragraph J or Paragraph K or Paragraph L or Paragraph M or Paragraph N or Paragraph O or Paragraph P or Paragraph Q, where during the third phase the antibody is provided once a week for up to four weeks (e.g., one, two, three, or four weeks), in a third dose amount described within any one of Paragraph I or Paragraph J or Paragraph K or Paragraph L or Paragraph M or Paragraph N or Paragraph O or Paragraph P or Paragraph Q, and where during the fourth phase the antibody is provided once a week until remission, in a fourth dose amount described within any one of Paragraph I or Paragraph J or Paragraph K or Paragraph L or Paragraph M or Paragraph N or Paragraph O or Paragraph P or Paragraph Q. In one embodiment, the first, second, third, and fourth amounts are the same. In one embodiment, three of the first, second, third, and fourth dose amounts not the same (i.e., three are the same and one is different). In one embodiment, two of the first, second, third, and fourth dose amounts are the same (i.e., two are the same and two are different). In one embodiment, the first, second, third, and fourth amounts are different. The administration times can independently be any described throughout the specification.

### First Phase, Three Times a Week

The method of treatment disclosed herein can comprise a first phase and a second phase, where during the first phase the antibody is provided three times a week for a duration of one week in a first dose amount, and where during the second phase the antibody is provided once a week in a second dose amount. In some embodiments, the second phase continues until remission. In some embodiment, the first and second dose amounts are the same. In one embodiment, the first and second dose amounts are different. The administration times can independently be any described throughout the specification.

The method of treatment disclosed herein can comprise a first phase and a second phase, where during the first phase the antibody is provided three times a week for a duration of one week, where the first dose amount in the first phase is described in Paragraph I or Paragraph J, and the subsequent two dose amounts are greater than the first dose amount and are described herein, and where during the second phase the antibody is provided once a week in a second dose amount. The method of treatment disclosed herein can comprise a first phase and a second phase, where during the first phase the antibody is provided three times a week for a duration of one week, where the first dose amount is between about 100 ng/kg and about 750 ng/kg, and the subsequent two dose amounts are greater than the first dose amount and are described herein, and where during the second phase the antibody is provided once a week in a second dose amount. The method of treatment disclosed herein can comprise a first phase and a second phase, where during the first phase the antibody is provided three times a week for a duration of one week, where the first dose amount is between about 600 ng/kg and about 750 ng/kg, and the subsequent two dose amounts are greater than the first dose amount and are described herein, and where during the second phase the antibody is provided once a week in a second dose amount. In some embodiments, the second phase continues until remission. In some embodiment, the first and second dose amounts are the same. In one embodiment, the first and second dose amounts are different. The administration times can independently be any described throughout the specification.

In one embodiment, the method of treatment can comprise a first phase and a second phase, where during the first phase the antibody is provided three times a week for a duration of one week in a dose amount described within any one of Paragraph F or Paragraph G or Paragraph H or Paragraph I or Paragraph J or Paragraph K or Paragraph L or Paragraph M for a first administration time, and where during the second phase the antibody is provided once a week in a dose amount described within Paragraph I or Paragraph J or Paragraph K or Paragraph L or Paragraph M or Paragraph N or Paragraph O or Paragraph P or Paragraph Q. In some embodiments, the second phase continues until remission. In some embodiment, the first and second dose amounts are the same. In one embodiment, the first and second dose amounts are different. The administration times can independently be any described throughout the specification.

In one embodiment, the method of treatment can comprise a first phase and a second phase, where during the first phase the antibody is provided three times a week for a duration of one week where the first dose amount in the first phase is described in Paragraph I or Paragraph J, and the subsequent two dose amounts are greater than the first dose amount and are described herein, for a first administration time, and where during the second phase the antibody is provided once a week in a dose amount described within Paragraph I or Paragraph J or Paragraph K or Paragraph L or Paragraph M or Paragraph N or Paragraph O or Paragraph P or Paragraph Q. In one embodiment, the method of treatment can comprise a first phase and a second phase, where during the first phase the antibody is provided three times a week for a duration of one week where the first dose amount is between about 100 ng/kg and about 750 ng/kg, and the subsequent two dose amounts are greater than the first dose amount and are described herein, for a first administration time, and where during the second phase the antibody is provided once a week in a dose amount described within Paragraph I or Paragraph J or Paragraph K or Paragraph L or Paragraph M or Paragraph N or Paragraph O or Paragraph P or Paragraph Q. In one embodiment, the method of treatment can comprise a first phase and a second phase, where during the first phase the antibody is provided three times a week for a duration of one week where the first dose amount is between about 600 ng/kg and about 750 ng/kg, and the subsequent two dose amounts are greater than the first dose amount and are described herein, for a first administration time, and where during the second phase the antibody is provided once a week in a dose amount described within Paragraph I or Paragraph J or Paragraph K or Paragraph L or Paragraph M or Paragraph N or Paragraph O or Paragraph P or Paragraph Q. In some embodiments, the second phase continues until remission. In some embodiment, the first and second dose amounts are the same. In one embodiment, the first and second dose amounts are different. The administration times can independently be any described throughout the specification.

The method of treatment disclosed herein can comprise a first phase and a second phase and a third phase, where during the first phase the antibody is provided three times a week for a duration of one week in a first dose amount, where during the second phase the antibody is provided three times a week for a duration of one week in a second dose amount, and where during the third phase the antibody is provided once a week in a third dose amount. In some embodiments, the third phase continues until remission. In one embodiment, the first, second, and third dose amounts are not the same. In one embodiment, the first, second, and third dose amounts are not the same (i.e., two are the same and one is different). The administration times can independently be any described throughout the specification.

The method of treatment disclosed herein can comprise a first phase and a second phase and a third phase, where during the first phase the antibody is provided three times a week for a duration of one week where the first dose amount in the first phase is described in Paragraph I or Paragraph J, and the subsequent two dose amounts are greater than the first dose amount and are described herein, where during the second phase the antibody is provided three times a week for a duration of one week in a second dose amount, and where during the third phase the antibody is provided once a week in a third dose amount. The method of treatment disclosed herein can comprise a first phase and a second phase and a third phase, where during the first phase the antibody is provided three times a week for a duration of one week, where the first dose amount is between about 100 ng/kg and about 750 ng/kg, and the subsequent two dose amounts are greater than the first dose amount and are described herein, where during the second phase the antibody is provided three times a week for a duration of one week in a second dose amount, and where during the third phase the antibody is provided once a week in a third dose amount. The method of treatment disclosed herein can comprise a first phase and a second phase and a third phase, where during the first phase the antibody is provided three times a week for a duration of one week where during the first phase the antibody is provided three times a week for a duration of one week, where the first dose amount is between about 600 ng/kg and about 750 ng/kg, and the subsequent two dose amounts are greater than the first dose amount and are described herein, where during the second phase the antibody is provided three times a week for a duration of one week in a second dose amount, and where during the third phase the antibody is provided once a week in a third dose amount. In some embodiments, the third phase continues until remission. In one embodiment, the first, second, and third dose amounts are not the same. In one embodiment, the first, second, and third dose amounts are not the same (i.e., two are the same and one is different). The administration times can independently be any described throughout the specification.

The method of treatment disclosed herein can comprise a first phase and a second phase and a third phase, where during the first phase the antibody is provided three times a week for a duration of one week, where during the first phase the antibody is provided three times a week for a duration of one week, where the first dose amount in the first phase is smaller than the subsequent two dose amounts in the first phase, where during the second phase the antibody is provided three times a week for a duration of one week in a second dose amount, and where during the third phase the antibody is provided once a week in a third dose amount. In some embodiments, the third phase continues until remission. In one embodiment, the first, second, and third dose amounts are not the same. In one embodiment, the first, second, and third dose amounts are not the same (i.e., two are the same and one is different). The administration times can independently be any described throughout the specification.

The method of treatment disclosed herein can comprise a first phase and a second phase and a third phase, where during the first phase the antibody is provided three times a week for a duration of one week, where during the first phase the antibody is provided three times a week for a duration of one week, where the first dose amount in the first phase is smaller than the subsequent two dose amounts in the first phase, where during the second phase the antibody is provided three times a week for a duration of one week in a second dose amount, and where during the third phase the antibody is provided once a week in a third dose amount. The method of treatment disclosed herein can comprise a first phase and a second phase and a third phase, where during the first phase the antibody is provided three times a week for a duration of one week, where the first dose amount is between about 100 ng/kg and about 750 ng/kg, and the subsequent two dose amounts are greater than the first dose amount and are described herein, where during the second phase the antibody is provided three times a week for a duration of one week in a second dose amount, and where during the third phase the antibody is provided once a week in a third dose amount.

The method of treatment disclosed herein can comprise a first phase and a second phase and a third phase, where during the first phase the antibody is provided three times a week for a duration of one week, where the first dose amount is between about 600 ng/kg and about 750 ng/kg, and the subsequent two dose amounts are greater than the first dose amount and are described herein, where during the second phase the antibody is provided three times a week for a duration of one week in a second dose amount, and where during the third phase the antibody is provided once a week in a third dose amount. In some embodiments, the third phase continues until remission. In one embodiment, the first, second, and third dose amounts are not the same. In one embodiment, the first, second, and third dose amounts are not the same (i.e., two are the same and one is different). The administration times can independently be any described throughout the specification.

In one embodiment, the method of treatment can comprise a first phase and a second phase and a third phase, where during the first phase the antibody is provided three times a week for a duration of one week, where the first dose amount in the first phase is disclosed in Paragraph J, and the subsequent two dose amounts in the first phase are disclosed in any one of Paragraph K or Paragraph L or Paragraph M or Paragraph N or Paragraph O or Paragraph P or Paragraph Q, or any combination thereof, where during the second phase the antibody is provided three times a week for a duration of one week in a dose amount described within any one of Paragraph F or Paragraph G or Paragraph H or Paragraph I or Paragraph J or Paragraph K or Paragraph L or Paragraph M for a second administration time, and where during the third phase the antibody is provided once a week in a dose amount described within Paragraph I or Paragraph J or Paragraph K or Paragraph L or Paragraph M or Paragraph N or Paragraph O or Paragraph P or Paragraph Q. In some embodiments, the third phase continues until remission. In one embodiment, the first, second, and third dose amounts are not the same. In one embodiment, the first, second, and third dose amounts are not the same (i.e., two are the same and one is different). The administration times can independently be any described throughout the specification.

In one embodiment, the method of treatment can comprise a first phase and a second phase and a third phase, where during the first phase the antibody is provided three times a week for a duration of one week, where the first dose amount in the first phase is about 750 ng/kg, and the subsequent two dose amounts in the first phase are disclosed in any one of Paragraph K or Paragraph L or Paragraph M or Paragraph N or Paragraph O or Paragraph P or Paragraph Q, or any combination thereof, where during the second phase the antibody is provided three times a week for a duration of one week in a dose amount described within any one of Paragraph F or Paragraph G or Paragraph H or Paragraph I or Paragraph J or Paragraph K or Paragraph L or Paragraph M for a second administration time, and where during the third phase the antibody is provided once a week in a dose amount described within Paragraph I or Paragraph J or Paragraph K or Paragraph L or Paragraph M or Paragraph N or Paragraph O or Paragraph P or Paragraph Q. In some embodiments, the third phase continues until remission. In one embodiment, the first, second, and third dose amounts are not the same. In one embodiment, the first, second, and third dose amounts are not the same (i.e., two are the same and one is different). The administration times can independently be any described throughout the specification.

In one embodiment, the method of treatment can comprise a first phase and a second phase and a third phase, where during the first phase the antibody is provided three times a week for a duration of one week, where the first dose amount in the first phase is between about 700 ng/kg and about 800 ng/kg, and the subsequent two dose amounts in the first phase are in Paragraphs K or L or M or N or O or P or Q for a first administration time, where during the second phase the antibody is provided three times a week for a duration of one week in a dose amount described within any one of Paragraph F or Paragraph G or Paragraph H or Paragraph I or Paragraph J or Paragraph K or Paragraph L or Paragraph M for a second administration time, and where during the third phase the antibody is provided once a week in a dose amount described within Paragraph I or Paragraph J or Paragraph K or Paragraph L or Paragraph M or Paragraph N or Paragraph O or Paragraph P or Paragraph Q. In one embodiment, the method of treatment can comprise a first phase and a second phase and a third phase, where during the first phase the antibody is provided three times a week for a duration of one week, where the first dose amount in the first phase is about 750 ng/kg, and the subsequent two dose amounts in the first phase are in Paragraphs K or L or M or N or O or P or Q for a first administration time, where during the second phase the antibody is provided three times a week for a duration of one week in a dose amount described within any one of Paragraph F or Paragraph G or Paragraph H or Paragraph I or Paragraph J or Paragraph K or Paragraph L or Paragraph M for a second administration time, and where during the third phase the antibody is provided once a week in a dose amount described within Paragraph I or Paragraph J or Paragraph K or Paragraph L or Paragraph M or Paragraph N or Paragraph O or Paragraph P or Paragraph Q. In some embodiments, the third phase continues until remission. In one embodiment, the first, second, and third dose amounts are not the same. In one embodiment, the first, second, and third dose amounts are not the same (i.e., two are the same and one is different). The administration times can independently be any described throughout the specification.

### VII. More Embodiments

In one embodiment, the method comprises providing the antibody once a week in a dose amount that is between about 1,150 ng/kg and about 1,450 ng/kg. In some embodiments, this method continues until the cancer (e.g., CD123-expressing cancer) is in remission (e.g., partial or complete). In one embodiment, the antibody is provided once a week in a dose amount of about 1,300 ng/kg.

In one embodiment, the method comprises providing the antibody once a week in a dose amount that is between about 2,200 ng/kg and about 2,400 ng/kg. In some embodiments, this method continues until the cancer (e.g., CD123-expressing cancer) is in remission (e.g., partial or complete). In one embodiment, the antibody is provided once a week in a dose amount of about 2,300 ng/kg.

In one embodiment, the method comprises providing the antibody once a week with a dose amount that is between about 3,750 ng/kg and about 4,250 ng/kg. In some embodiments, this method continues until the cancer (e.g., CD123-expressing cancer) is in remission (e.g., partial or complete). In one embodiment, the antibody is provided once a week in a dose amount of about 4,000 ng/kg.

In one embodiment, the method comprises providing the antibody once a week with a dose amount that is between about 6,500 ng/kg and about 7,500 ng/kg. In some embodiments, this method continues until the cancer (e.g., CD123-expressing cancer) is in remission (e.g., partial or complete). In one embodiment, the antibody is provided once a week in a dose amount of about 7,000 ng/kg.

In one embodiment, the method comprises providing the antibody once a week with a dose amount that is between about 11,000 ng/kg and about 13,000 ng/kg. In some embodiments, this method continues until the cancer (e.g., CD123-expressing cancer) is in remission (e.g., partial or complete). In one embodiment, the antibody is provided once a week in a dose amount of about 12,000 ng/kg.

In one embodiment, the method comprises providing the antibody once a week with a dose amount that is between about 19,000 ng/kg and about 21,000 ng/kg. In some embodiments, this method continues until the cancer (e.g., CD123-expressing cancer) is in remission (e.g., partial or complete). In one embodiment, the antibody is provided once a week in a dose amount of about 20,000 ng/kg.

In one embodiment, the method comprises providing the antibody once a week with a dose amount that is between about 34,000 ng/kg and about 36,000 ng/kg. In some embodiments, this method continues until the cancer (e.g., CD123-expressing cancer) is in remission (e.g., partial or complete). In one embodiment, the antibody is provided once a week in a dose amount of about 35,000 ng/kg.

In one embodiment, the method of treatment comprises a first phase and a second phase, where during the first phase the antibody is provided once a week for a duration of up to four weeks (e.g., one, two, three, or four weeks), with a first dose amount, and where during the second phase the antibody is provided once a week until remission in a second dose amount. The first dose amount and the second dose amount can include any one of doses referenced in the paragraphs and rows of Table A. For example, the dose amounts in row 1 of Table A include a first dose amount that can be any dose amount in Paragraph K and a second dose amount can be any dose amount in Paragraph L. The administration times can independently be any described throughout the specification.

TABLE A

| | First Dose | Second Dose |
|---|---|---|
| | amount | amount |
| 1 | Paragraph K | Paragraph L |
| 2 | Paragraph K | Paragraph M |
| 3 | Paragraph K | Paragraph N |
| 4 | Paragraph K | Paragraph O |
| 5 | Paragraph K | Paragraph P |
| 6 | Paragraph K | Paragraph Q |
| 7 | Paragraph L | Paragraph M |
| 8 | Paragraph L | Paragraph N |
| 9 | Paragraph L | Paragraph O |
| 10 | Paragraph L | Paragraph P |
| 11 | Paragraph L | Paragraph Q |
| 12 | Paragraph M | Paragraph N |
| 13 | Paragraph M | Paragraph O |
| 14 | Paragraph M | Paragraph P |
| 15 | Paragraph M | Paragraph Q |
| 16 | Paragraph N | Paragraph O |
| 17 | Paragraph N | Paragraph P |
| 18 | Paragraph N | Paragraph Q |
| 19 | Paragraph O | Paragraph P |
| 20 | Paragraph O | Paragraph Q |
| 21 | Paragraph P | Paragraph Q |
| 22 | Paragraph J | Paragraph K |
| 23 | Paragraph J | Paragraph L |
| 24 | Paragraph J | Paragraph M |
| 25 | Paragraph J | Paragraph N |

In one embodiment, the method comprises a first phase and a second phase, where during the first phase the antibody is provided once a week, for a duration of one, two, three, or four weeks in a first dose amount of between about 1,150 ng/kg and about 1,450 ng/kg, and where during the second phase the antibody is provided once a week until remission in a second dose amount of between about 2,200 ng/kg and about 2,400 ng/kg. The administration times can independently be any described throughout the specification.

In one embodiment, the method comprises a first phase and a second phase, where during the first phase the antibody is provided once a week for one week in a first dose amount of between about 1,150 ng/kg and about 1,450 ng/kg, and where during the second phase the antibody is provided once a week until remission in a second dose amount of between about 2,200 ng/kg and about 2,400 ng/kg. The administration times can independently be any described throughout the specification.

In one embodiment, the method comprises a first phase and a second phase, where during the first phase the antibody is provided once a week for one week, in a first dose amount of about 1,300 ng/kg, and where during the second phase the antibody is provided once a week until remission in a second dose amount of about 2,300 ng/kg. The administration times can independently be any described throughout the specification.

In one embodiment, the method comprises a first phase and a second phase, where during the first phase the antibody is provided once a week, for a duration of one, two, three, or four weeks in a first dose amount of between about 600 ng/kg and about 750 ng/kg, and where during the second phase the antibody is provided once a week until remission in a second dose amount of between about 1,150 ng/kg and about 1,450 ng/kg. The administration times can independently be any described throughout the specification.

In one embodiment, the method comprises a first phase and a second phase, where during the first phase the antibody is provided once a week for one week in a first dose amount of between about 600 ng/kg and about 750 ng/kg, and where during the second phase the antibody is provided once a week until remission in a second dose amount of between about 1,150 ng/kg and about 1,450 ng/kg. The administration times can independently be any described throughout the specification.

In one embodiment, the method comprises a first phase and a second phase, where during the first phase the antibody is provided once a week for one week, in a first dose amount of about 750 ng/kg, and where during the second phase the antibody is provided once a week until remission in a second dose amount of about 1,300 ng/kg. The administration times can independently be any described throughout the specification.

In one embodiment, the method comprises a first phase and a second phase, where during the first phase the antibody is provided once a week, for a duration of one, two, three, or four weeks in a first dose amount of between about 600 ng/kg and about 750 ng/kg, and where during the second phase the antibody is provided once a week until remission in a second dose amount of between about 2,200 ng/kg and about 2,400 ng/kg. The administration times can independently be any described throughout the specification.

In one embodiment, the method comprises a first phase and a second phase, where during the first phase the antibody is provided once a week for one week in a first dose amount of between about 600 ng/kg and about 750 ng/kg, and where during the second phase the antibody is provided once a week until remission in a second dose amount of between about 2,200 ng/kg and about 2,400 ng/kg. The administration times can independently be any described throughout the specification.

In one embodiment, the method comprises a first phase and a second phase, where during the first phase the antibody is provided once a week for one week, in a first dose amount of about 750 ng/kg, and where during the second phase the antibody is provided once a week until remission in a second dose amount of about 2,300 ng/kg. The administration times can independently be any described throughout the specification.

In one embodiment, the method comprises a first phase and a second phase, where during the first phase the antibody is provided once a week, for a duration of one, two, three, or four weeks in a first dose amount of between about 600 ng/kg and about 750 ng/kg, and where during the second phase the antibody is provided once a week until remission in a second dose amount of between about 3,750 ng/kg and about 4,250 ng/kg. The administration times can independently be any described throughout the specification.

In one embodiment, the method comprises a first phase and a second phase, where during the first phase the antibody is provided once a week for one week in a first dose amount of between about 600 ng/kg and about 750 ng/kg, and where during the second phase the antibody is provided once a week until remission in a second dose amount of between about 3,750 ng/kg and about 4,250 ng/kg. The administration times can independently be any described throughout the specification.

In one embodiment, the method comprises a first phase and a second phase, where during the first phase the antibody is provided once a week for one week, in a first dose amount of about 750 ng/kg, and where during the second phase the antibody is provided once a week until remission in a second dose amount of about 4,000 ng/kg. The administration times can independently be any described throughout the specification.

In one embodiment, the method comprises a first phase and a second phase, where during the first phase the antibody is provided once a week, for a duration of one, two, three, or four weeks in a first dose amount of between about 600 ng/kg and about 750 ng/kg, and where during the second phase the antibody is provided once a week until remission in a second dose amount of between about 6,000 ng/kg and about 8,000 ng/kg. The administration times can independently be any described throughout the specification.

In one embodiment, the method comprises a first phase and a second phase, where during the first phase the antibody is provided once a week for one week in a first dose amount of between about 600 ng/kg and about 750 ng/kg, and where during the second phase the antibody is provided once a week until remission in a second dose amount of between about 6,000 ng/kg and about 8,000 ng/kg. The administration times can independently be any described throughout the specification.

In one embodiment, the method comprises a first phase and a second phase, where during the first phase the antibody is provided once a week for one week, in a first dose amount of about 750 ng/kg, and where during the second phase the antibody is provided once a week until remission in a second dose amount of about 7,000 ng/kg. The administration times can independently be any described throughout the specification.

In one embodiment, the method comprises a first phase and a second phase, where during the first phase the antibody is provided once a week, for a duration of one, two, three, or four weeks in a first dose amount of between about 600 ng/kg and about 750 ng/kg, and where during the second phase the antibody is provided once a week until remission in a second dose amount of between about 11,000 ng/kg and about 13,000 ng/kg. The administration times can independently be any described throughout the specification.

In one embodiment, the method comprises a first phase and a second phase, where during the first phase the antibody is provided once a week for one week in a first dose amount of between about 600 ng/kg and about 750 ng/kg, and where during the second phase the antibody is provided once a week until remission in a second dose amount of between about 11,000 ng/kg and about 13,000 ng/kg. The administration times can independently be any described throughout the specification.

In one embodiment, the method comprises a first phase and a second phase, where during the first phase the antibody is provided once a week for one week, in a first dose amount of about 750 ng/kg, and where during the second phase the antibody is provided once a week until remission in a second dose amount of about 12,000 ng/kg. The administration times can independently be any described throughout the specification.

### Three Phases

The methods of treatment described herein comprises a first phase, second phase, and a third phase, where during the first phase the antibody is provided once a week for a duration of up to four weeks (e.g., one, two, three, or four weeks) with a first dose amount, where during the second phase, the antibody is provided once a week for a duration of up to four weeks (e.g., one, two, three, or four weeks) with a second dose amount, and where during the third phase the antibody is provided once a week until remission with a third dose amount. The first, second, and third dose amounts can include any one of the doses referenced in the paragraphs and rows of Table B. For example, the doses referring to row 1 of Table B includes a first dose amount that can be any dose amount in Paragraph K, a second dose amount can be any dose amount in Paragraph L, and a third dose amount can be any dose amount in Paragraph M. The administration times can independently be any described throughout the specification.

TABLE B

| | First Dose | Second Dose | Third Dose |
|---|---|---|---|
| | amount | amount | amount |
| 1 | Paragraph K | Paragraph L | Paragraph M |
| 2 | Paragraph K | Paragraph L | Paragraph N |
| 3 | Paragraph K | Paragraph L | Paragraph O |
| 4 | Paragraph K | Paragraph L | Paragraph P |
| 5 | Paragraph K | Paragraph L | Paragraph Q |
| 6 | Paragraph K | Paragraph M | Paragraph N |
| 7 | Paragraph K | Paragraph M | Paragraph O |
| 8 | Paragraph K | Paragraph M | Paragraph P |
| 9 | Paragraph K | Paragraph M | Paragraph Q |
| 10 | Paragraph K | Paragraph N | Paragraph O |
| 11 | Paragraph K | Paragraph N | Paragraph P |
| 12 | Paragraph K | Paragraph N | Paragraph Q |
| 13 | Paragraph K | Paragraph O | Paragraph P |
| 14 | Paragraph K | Paragraph O | Paragraph Q |
| 15 | Paragraph K | Paragraph P | Paragraph Q |
| 16 | Paragraph L | Paragraph M | Paragraph N |
| 17 | Paragraph L | Paragraph M | Paragraph O |
| 18 | Paragraph L | Paragraph M | Paragraph P |
| 19 | Paragraph L | Paragraph M | Paragraph Q |
| 20 | Paragraph L | Paragraph N | Paragraph O |
| 21 | Paragraph L | Paragraph N | Paragraph P |
| 22 | Paragraph L | Paragraph N | Paragraph Q |
| 23 | Paragraph L | Paragraph O | Paragraph P |
| 24 | Paragraph L | Paragraph O | Paragraph Q |
| 25 | Paragraph L | Paragraph P | Paragraph Q |
| 26 | Paragraph M | Paragraph N | Paragraph O |
| 27 | Paragraph M | Paragraph N | Paragraph P |
| 28 | Paragraph M | Paragraph N | Paragraph Q |
| 29 | Paragraph M | Paragraph O | Paragraph P |
| 30 | Paragraph M | Paragraph O | Paragraph Q |
| 31 | Paragraph M | Paragraph P | Paragraph Q |
| 32 | Paragraph N | Paragraph O | Paragraph P |
| 33 | Paragraph N | Paragraph O | Paragraph Q |
| 34 | Paragraph N | Paragraph P | Paragraph Q |
| 35 | Paragraph O | Paragraph P | Paragraph Q |
| 36 | Paragraph I | Paragraph I | Paragraph J |
| 37 | Paragraph I | Paragraph J | Paragraph K |
| 38 | Paragraph I | Paragraph J | Paragraph L |
| 39 | Paragraph J | Paragraph J | Paragraph K |
| 40 | Paragraph J | Paragraph J | Paragraph L |
| 41 | Paragraph J | Paragraph J | Paragraph M |
| 42 | Paragraph J | Paragraph K | Paragraph L |
| 43 | Paragraph J | Paragraph K | Paragraph M |
| 44 | Paragraph J | Paragraph L | Paragraph M |
| 45 | Paragraph J | Paragraph L | Paragraph N |

In one embodiment, the method comprises a first phase, a second phase, and a third phase, where during the first phase the antibody is provided once a week for a duration of up to four weeks (e.g., one, two, three, or four weeks) in a first dose amount of between about 1,150 ng/kg and about 1,450 ng/kg, where during the second phase the antibody is provided once a week for a duration of up to four weeks (e.g., one, two, three, or four weeks) in a second dose amount of between about 2,200 ng/kg and about 2,400 ng/kg, and where during the third phase the antibody is provided once a week until remission in a third dose amount of between about 3,750 ng/kg and about 4,250 ng/kg. The administration times can independently be any described throughout the specification.

In one embodiment, the method comprises a first phase, a second phase, and a third phase, where during the first phase the antibody is provided once a week for one week in a first dose amount of between about 1,150 ng/kg and about 1,450 ng/kg, where during the second phase the antibody is provided once a week for two weeks in a second dose amount of between about 2,200 ng/kg and about 2,400 ng/kg, and where during the third phase the antibody is provided once a week until remission in a third dose amount of between about 3,750 ng/kg and about 4,250 ng/kg. The administration times can independently be any described throughout the specification.

In one embodiment, the method comprises a first phase, a second phase, and a third phase, where during the first phase the antibody is provided once a week for one week in a first dose amount of about 1,300 ng/kg, where during the second phase the antibody is provided once a week for a duration of two weeks in a second dose amount of about 2,300 ng/kg, and where during the third phase the antibody is provided once a week until remission in a third dose amount of about 4,000 ng/kg. The administration times can independently be any described throughout the specification.

In one embodiment, the method comprises a first phase, a second phase, and a third phase, where during the first phase the antibody is provided once a week for a duration of up to four weeks (e.g., one, two, three, or four weeks) in a first dose amount is about 750 ng/kg, where during the second phase the antibody is provided once a week for a duration of up to four weeks (e.g., one, two, three, or four weeks) in a second dose amount of between about 1,150 ng/kg and about 1,450 ng/kg, and where during the third phase the antibody is provided once a week until remission in a third dose amount of between about 3,750 ng/kg and about 4,250 ng/kg. The administration times can independently be any described throughout the specification.

In one embodiment, the method comprises a first phase, a second phase, and a third phase, where during the first phase the antibody is provided once a week for one week in a first dose amount is about 750 ng/kg, where during the second phase the antibody is provided once a week for two weeks in a second dose amount of between about 1,000 ng/kg and about 1,400 ng/kg, and where during the third phase the antibody is provided once a week until remission in a third dose amount of between about 1,150 ng/kg and about 1,450 ng/kg. The administration times can independently be any described throughout the specification.

In one embodiment, the method comprises a first phase, a second phase, and a third phase, where during the first phase the antibody is provided once a week for one week in a first dose amount of about 750 ng/kg, where during the second phase the antibody is provided once a week for a duration of two weeks in a second dose amount of about 1,125 ng/kg, and where during the third phase the antibody is provided once a week until remission in a third dose amount of about 1,300 ng/kg. The administration times can independently be any described throughout the specification.

### Four Phases

The methods of treatment described herein comprises a first phase, second phase, a third phase, and fourth phase where during the first phase the antibody is provided once a week for a duration of up to four weeks (e.g., one, two, three, or four weeks) with a first dose amount, where during the second phase, the antibody is provided once a week for a duration of up to four weeks (e.g., one, two, three, or four weeks) with a second dose amount, where during the third phase the antibody is provided once a week for a duration of up to four weeks (e.g., one, two, three, or four weeks) with a third dose amount, and where during the fourth phase the antibody is provided once a week until remission with a fourth dose amount. The first, second, third, and fourth dose amounts can include any one of the doses referenced in the paragraphs and rows of Table C. For example, the doses referring to row 1 of Table C includes a first dose amount that can be any dose amount in Paragraph K, a second dose amount can be any dose amount in Paragraph L, a third dose amount can be any dose amount in Paragraph M, and a fourth dose amount can be any dose amount in Paragraph N. The administration times can independently be any described throughout the specification.

TABLE C

| | First Dose | Second Dose | Third Dose | Fourth Dose |
|---|---|---|---|---|
| | amount | amount | amount | amount |
| 1 | Paragraph K | Paragraph L | Paragraph M | Paragraph N |
| 2 | Paragraph K | Paragraph L | Paragraph M | Paragraph O |
| 3 | Paragraph K | Paragraph L | Paragraph M | Paragraph P |
| 4 | Paragraph K | Paragraph L | Paragraph M | Paragraph Q |
| 5 | Paragraph K | Paragraph L | Paragraph N | Paragraph O |
| 6 | Paragraph K | Paragraph L | Paragraph N | Paragraph P |
| 7 | Paragraph K | Paragraph L | Paragraph N | Paragraph Q |
| 8 | Paragraph K | Paragraph L | Paragraph O | Paragraph P |
| 9 | Paragraph K | Paragraph L | Paragraph O | Paragraph Q |
| 10 | Paragraph K | Paragraph L | Paragraph P | Paragraph Q |
| 11 | Paragraph K | Paragraph M | Paragraph N | Paragraph O |
| 12 | Paragraph K | Paragraph M | Paragraph N | Paragraph P |
| 13 | Paragraph K | Paragraph M | Paragraph N | Paragraph Q |
| 14 | Paragraph K | Paragraph M | Paragraph O | Paragraph P |
| 15 | Paragraph K | Paragraph M | Paragraph O | Paragraph Q |
| 16 | Paragraph K | Paragraph M | Paragraph P | Paragraph Q |
| 17 | Paragraph K | Paragraph N | Paragraph O | Paragraph P |
| 18 | Paragraph K | Paragraph N | Paragraph O | Paragraph Q |
| 19 | Paragraph K | Paragraph N | Paragraph P | Paragraph Q |
| 20 | Paragraph K | Paragraph O | Paragraph P | Paragraph Q |
| 21 | Paragraph L | Paragraph M | Paragraph N | Paragraph O |
| 22 | Paragraph L | Paragraph M | Paragraph N | Paragraph P |
| 23 | Paragraph L | Paragraph M | Paragraph N | Paragraph Q |
| 24 | Paragraph L | Paragraph M | Paragraph O | Paragraph P |
| 25 | Paragraph L | Paragraph M | Paragraph O | Paragraph Q |
| 26 | Paragraph L | Paragraph M | Paragraph P | Paragraph Q |
| 27 | Paragraph M | Paragraph N | Paragraph O | Paragraph P |
| 28 | Paragraph M | Paragraph N | Paragraph O | Paragraph Q |
| 29 | Paragraph M | Paragraph N | Paragraph P | Paragraph Q |
| 30 | Paragraph M | Paragraph O | Paragraph P | Paragraph Q |
| 31 | Paragraph N | Paragraph O | Paragraph P | Paragraph Q |
| 32 | Paragraph I | Paragraph J | Paragraph J | Paragraph K |
| 33 | Paragraph I | Paragraph J | Paragraph K | Paragraph L |
| 34 | Paragraph J | Paragraph J | Paragraph J | Paragraph K |
| 35 | Paragraph J | Paragraph J | Paragraph K | Paragraph K |
| 36 | Paragraph J | Paragraph K | Paragraph L | Paragraph M |
| 37 | Paragraph J | Paragraph K | Paragraph L | Paragraph N |
| 38 | Paragraph J | Paragraph K | Paragraph L | Paragraph O |
| 39 | Paragraph J | Paragraph K | Paragraph L | Paragraph O |
| 40 | Paragraph J | Paragraph K | Paragraph M | Paragraph N |
| 41 | Paragraph J | Paragraph K | Paragraph M | Paragraph O |
| 42 | Paragraph J | Paragraph L | Paragraph M | Paragraph N |
| 43 | Paragraph J | Paragraph L | Paragraph M | Paragraph O |
| 44 | Paragraph J | Paragraph L | Paragraph N | Paragraph O |

In one embodiment, the method comprises a first phase, a second phase, a third phase, and a fourth phase, where during the first phase the antibody is provided once a week for a duration of up to four weeks (e.g., one, two, three, or four weeks), with a first dose amount of between about 1,150 ng/kg and about 1,450 ng/kg, where during the second phase the antibody is provided once a week for a duration of up to four weeks (e.g., one, two, three, or four weeks) with a second dose amount of between about 2,200 ng/kg and about 2,400 ng/kg, where during the third phase the antibody is provided once a week for a duration of up to four weeks (e.g., one, two, three, or four weeks), with a third dose amount is between about 3,750 ng/kg and about 4,250 ng/kg, and where during the fourth phase the antibody is provided once a week until remission, with a fourth dose amount of between about 6,500 ng/kg and about 7,500 ng/kg. The administration times can independently be any described throughout the specification.

In one embodiment, the method comprises a first phase, a second phase, a third phase, and a fourth phase, where during the first phase the antibody is provided once a week for one week, with a first dose amount of between about 1,150 ng/kg and about 1,450 ng/kg, where during the second phase the antibody is provided once a week for one week with a second dose amount of between about 2,200 ng/kg and about 2,400 ng/kg, where during the third phase the antibody is provided once a week for two weeks, with a third dose amount is between about 3,750 ng/kg and about 4,250 ng/kg, and where during the fourth phase the antibody is provided once a week until remission, with a fourth dose amount of between about 6,500 ng/kg and about 7,500 ng/kg. The administration times can independently be any described throughout the specification.

In one embodiment, the method comprises a first phase, a second phase, a third phase, and a fourth phase, where during the first phase the antibody is provided once a week for one week, where the first dose amount is about 1,300 ng/kg, where during the second phase the antibody is provided once a week for one week, where the second dose amount is about 2,300 ng/kg, where during the third phase the antibody is provided once a week for two weeks, where the third dose amount is about 4,000 ng/kg, where during the fourth phase the antibody is provided until remission, where the fourth dose amount is about 7,000 ng/kg. The administration times can independently be any described throughout the specification.

In one embodiment, the method comprises a first phase, a second phase, a third phase, and a fourth phase, where during the first phase the antibody is provided once a week for a duration of up to four weeks (e.g., one, two, three, or four weeks), where the first dose amount is between about 1,150 ng/kg and about 1,450 ng/kg, where during the second phase the antibody is provided once a week for a duration of up to four weeks (e.g., one, two, three, \ where the second dose amount is between about 3,750 ng/kg and about 4,250 ng/kg, where during the third phase the antibody is provided once a week for a duration of up to four weeks (e.g., one, two, three, or four weeks), where the third dose amount is between about 6,500 ng/kg and about 7,500 ng/kg, where during the fourth phase the antibody is provided once a week until remission, where the fourth dose amount is between about 11,000 ng/kg and about 13,000 ng/kg. The administration times can independently be any described throughout the specification.

In one embodiment, the method comprises a first phase, a second phase, a third phase, and a fourth phase, where during the first phase the antibody is provided once a week for one week, where the first dose amount is between about 1,150 ng/kg and about 1,450 ng/kg, where during the second phase the antibody is provided once a week for one week, where the second dose amount is between about 3,750 ng/kg and about 4,250 ng/kg, where during the third phase the antibody is provided once a week for one week, where the third dose amount is between about 6,500 ng/kg and about 7,500 ng/kg, where during the fourth phase the antibody is provided once a week until remission, where the fourth dose amount is between about 11,000 ng/kg and about 13,000 ng/kg. The administration times can independently be any described throughout the specification.

In one embodiment, the method comprises a first phase, a second phase, a third phase, and a fourth phase, where during the first phase the antibody is provided once a week for one week, where the first dose amount is about 1,300 ng/kg, where during the second phase the antibody is provided once a week for one week, where the second dose amount is about 4,000 ng/kg, where during the third phase the antibody is provided once a week for one week, where the third dose amount is about 7,000 ng/kg, where during the fourth phase the antibody is provided once a week until remission, where the fourth dose amount is about 12,000 ng/kg. The administration times can independently be any described throughout the specification.

In one embodiment, the method comprises a first phase, a second phase, a third phase, and a fourth phase, where during the first phase the antibody is provided once a week for a duration of up to four weeks (e.g., one, two, three, or four weeks), with a first dose amount of about 750 ng/kg, where during the second phase the antibody is provided once a week for a duration of up to four weeks (e.g., one, two, three, or four weeks) with a second dose amount of between about 1,000 ng/kg and about 1,400 ng/kg, where during the third phase the antibody is provided once a week for a duration of up to four weeks (e.g., one, two, three, or four weeks), with a third dose amount is between about 1,500 ng/kg and about 1,900 ng/kg, and where during the fourth phase the antibody is provided once a week until remission, with a fourth dose amount of between about 2,200 ng/kg and about 2,400 ng/kg. The administration times can independently be any described throughout the specification.

In one embodiment, the method comprises a first phase, a second phase, a third phase, and a fourth phase, where during the first phase the antibody is provided once a week for one week, with a first dose amount of about 750 ng/kg, where during the second phase the antibody is provided once a week for one week with a second dose amount of between about 1,000 ng/kg and about 1,400 ng/kg, where during the third phase the antibody is provided once a week for two weeks, with a third dose amount is between about 1,500 ng/kg and about 1,900 ng/kg, and where during the fourth phase the antibody is provided once a week until remission, with a fourth dose amount of between about 2,200 ng/kg and about 2,400 ng/kg. The administration times can independently be any described throughout the specification.

In one embodiment, the method comprises a first phase, a second phase, a third phase, and a fourth phase, where during the first phase the antibody is provided once a week for one week, where the first dose amount is about 750 ng/kg, where during the second phase the antibody is provided once a week for one week, where the second dose amount is about 1,125 ng/kg, where during the third phase the antibody is provided once a week for two weeks, where the third dose amount is about 1,725 ng/kg, where during the fourth phase the antibody is provided until remission, where the fourth dose amount is about 2,300 ng/kg. The administration times can independently be any described throughout the specification.

In one embodiment, the method comprises a first phase and a second phase, where during the first phase the antibody is provided two times a week or three times a week or four times a week, for a duration of up to four weeks (e.g., one, two, three, or four weeks) with a first dose amount, where during the second phase, the antibody is provided once a week until remission with a second dose amount. Combinations of the first phase dosage regimen and the first dosing amount, with the second dose amounts are referenced in the paragraphs and rows of Table D. For example, one method of treatment can comprise a first phase and a second phase, where during the first phase the antibody is provided according to row 5, column ii) (where the first dose amount can be any dose amount in Paragraph J), for a duration of up to four weeks (e.g., one, two, three, or four weeks), where during the second phase, the antibody is provided once a week until remission with a second dose amount according to row 5, column iv) (where the first dose amount can be any dose amount in Paragraph K). In another example, one method of treatment can comprise a first phase and a second phase, where during the first phase the antibody is provided according to row 2, column i) (where the first dose amount can be any dose amount in Paragraph H), for a duration of up to four weeks (e.g., one, two, three, or four weeks), where during the second phase, the antibody is provided once a week until remission with a second dose amount according to row 2, column iv) (where the first dose amount can be any dose amount in Paragraph K). The administration times can independently be any described throughout the specification.

TABLE D

| | i) | ii) | iii) | |
|---|---|---|---|---|
| | Two Times | Three Times | Four Times | iv) |
| | A Week | A Week | A Week | Second |
| | Dosage | Dosage | Dosage | Dose |
| | Regimen | Regimen | Regimen | amount |
| 1 | Paragraph H | Paragraph H | Paragraph G | Paragraph J |
| 2 | Paragraph H | Paragraph H | Paragraph G | Paragraph K |
| 3 | Paragraph H | Paragraph H | Paragraph G | Paragraph L |
| 4 | Paragraph H | Paragraph H | Paragraph G | Paragraph M |
| 5 | Paragraph J | Paragraph H | Paragraph H | Paragraph K |
| 6 | Paragraph J | Paragraph H | Paragraph H | Paragraph L |
| 7 | Paragraph J | Paragraph H | Paragraph H | Paragraph M |
| 8 | Paragraph K | Paragraph J | Paragraph I | Paragraph K |
| 9 | Paragraph K | Paragraph J | Paragraph I | Paragraph L |
| 10 | Paragraph K | Paragraph J | Paragraph I | Paragraph M |
| 11 | Paragraph K | Paragraph J | Paragraph I | Paragraph N |
| 12 | Paragraph K | Paragraph J | Paragraph I | Paragraph O |
| 13 | Paragraph L | Paragraph K | Paragraph K | Paragraph K |
| 14 | Paragraph L | Paragraph K | Paragraph K | Paragraph L |
| 15 | Paragraph L | Paragraph K | Paragraph K | Paragraph M |
| 16 | Paragraph M | Paragraph L | Paragraph L | Paragraph K |
| 17 | Paragraph M | Paragraph L | Paragraph L | Paragraph L |
| 18 | Paragraph M | Paragraph L | Paragraph L | Paragraph M |

In one embodiment, the method comprises a first phase and a second phase, where the combination of the first phase dosage regimen and the first dosing amount, with the second dose amount are according to a row in Table D, where the first dosing regimen occurs two times in the first phase, and where the second phase is provided once a week until remission. The administration times can independently be any described throughout the specification.

In one embodiment, the method comprises a first phase and a second phase, where the first phase is provided three times a week for two weeks, where the first dose amount is between about 225 ng/kg and about 275 ng/kg, where the second phase is provided once a week until remission, where the second dose amount is between about 700 ng/kg and about 800 ng/kg. The administration times can independently be any described throughout the specification.

In one embodiment, the method comprises a first phase and a second phase, where the first phase is provided three times a week for two weeks, where the first dose amount is between about 225 ng/kg and about 275 ng/kg, where the second phase is provided once a week until remission, where the second dose amount is between about 740 ng/kg and about 780 ng/kg. The administration times can independently be any described throughout the specification.

In one embodiment, the method comprises a first phase and a second phase, where the first phase is provided three times a week for two weeks, where the first dose amount is about 250 ng/kg, where the second phase is provided a once a week until remission, where the second dose amount is about 750 ng/kg. The administration times can independently be any described throughout the specification.

In one embodiment, the method comprises a first phase and a second phase, where the first phase is provided three times a week for two weeks, where the first dose amount is between about 400 ng/kg and about 450 ng/kg, where the second phase is provided once a week until remission, where the second dose amount is between about 1,150 ng/kg and about 1,450 ng/kg. The administration times can independently be any described throughout the specification.

In one embodiment, the method comprises a first phase and a second phase, where the first phase is provided three times a week for two weeks, where the first dose amount is about 430 ng/kg, where the second phase is provided a once a week until remission, where the second dose amount is about 1,300 ng/kg. The administration times can independently be any described throughout the specification.

In one embodiment, the method comprises a first phase and a second phase, where the first phase is provided three times a week for two weeks, where the first dose amount is between about 700 ng/kg and about 800 ng/kg, where the second phase is provided once a week until remission, where the second dose amount is between about 2,200 ng/kg and about 2,400 ng/kg. The administration times can independently be any described throughout the specification.

In one embodiment, the method comprises a first phase and a second phase, where the first phase is provided three times a week for two weeks, where the first dose amount is about 766 ng/kg, where the second phase is provided a once a week until remission, where the second dose amount is about 2,300 ng/kg. The administration times can independently be any described throughout the specification.

In one embodiment, the method comprises a first phase and a second phase, where the first phase is provided three times a week for two weeks, where the first dose amount is between about 1,150 ng/kg and about 1,450 ng/kg, where the second phase is provided once a week until remission, where the second dose amount is between about 3,750 ng/kg and about 4,250 ng/kg. The administration times can independently be any described throughout the specification.

In one embodiment, the method comprises a first phase and a second phase, where the first phase is provided three times a week for two weeks, where the first dose amount is about 1,133 ng/kg, where the second phase is provided a once a week until remission, where the second dose amount is about 4,000 ng/kg. The administration times can independently be any described throughout the specification.

In one embodiment, the method comprises a first phase and a second phase, where the first phase is provided three times a week for two weeks, where the first dose amount is between about 2,000 ng/kg and about 2,600 ng/kg, where the second phase is provided once a week until remission, where the second dose amount is between about 6,000 ng/kg and about 8,000 ng/kg. The administration times can independently be any described throughout the specification.

In one embodiment, the method comprises a first phase and a second phase, where the first phase is provided three times a week for two weeks, where the first dose amount is about 2,300 ng/kg, where the second phase is provided a once a week until remission, where the second dose amount is about 7,000 ng/kg. The administration times can independently be any described throughout the specification.

In one embodiment, the method comprises a first phase and a second phase, where the first phase is provided three times a week for two weeks, where the first dose amount is between about 3,000 ng/kg and about 5,000 ng/kg, where the second phase is provided once a week until remission, where the second dose amount is between about 11,000 ng/kg and about 13,000 ng/kg. The administration times can independently be any described throughout the specification.

In one embodiment, the method comprises a first phase and a second phase, where the first phase is provided three times a week for two weeks, where the first dose amount is about 4,000 ng/kg, where the second phase is provided a once a week until remission, where the second dose amount is about 12,000 ng/kg. The administration times can independently be any described throughout the specification.

In one embodiment, the method comprises a first phase and a second phase, where the first phase is provided three times a week for two weeks, where the first dose amount is between about 6,000 ng/kg and about 7,000 ng/kg, where the second phase is provided once a week until remission, where the second dose amount is between about 19,000 ng/kg and about 21,000 ng/kg. The administration times can independently be any described throughout the specification.

In one embodiment, the method comprises a first phase and a second phase, where the first phase is provided three times a week for two weeks, where the first dose amount is about 6,777 ng/kg, where the second phase is provided a once a week until remission, where the second dose amount is about 20,000 ng/kg. The administration times can independently be any described throughout the specification.

In one embodiment, the method comprises a first phase and a second phase, where the first phase is provided three times a week for two weeks, where the first dose amount is between about 11,250 ng/kg and about 12,000 ng/kg, where the second phase is provided once a week until remission, where the second dose amount is between about 31,000 ng/kg and about 38,000 ng/kg. The administration times can independently be any described throughout the specification.

In one embodiment, the method comprises a first phase and a second phase, where the first phase is provided three times a week for two weeks, where the first dose amount is about 11,667 ng/kg, where the second phase is provided a once a week until remission, where the second dose amount is about 35,000 ng/kg. The administration times can independently be any described throughout the specification.

In one embodiment, the method of treatment comprises a first phase and a second phase and a third phase, where during the first phase the antibody is provided three times a week for a duration of one week, where the first dose amount in the first phase is between about 700 ng/kg and about 800 ng/kg, and the subsequent two dose amounts in the first phase are between about 700 ng/kg and about 800 ng/kg, for a first administration time, where during the second phase the antibody is provided three times a week for a duration of one week in a dose amount of between about 700 ng/kg and about 800 ng/kg for a second administration time, and where during the third phase the antibody is provided once a week until remission in a dose amount of between about 2,200 ng/kg and 2,400 ng/kg.

In one embodiment, the method of treatment comprises a first phase and a second phase and a third phase, where during the first phase the antibody is provided three times a week for a duration of one week, where the first dose amount in the first phase is between about 740 ng/kg and about 760 ng/kg, and the subsequent two dose amounts in the first phase are between about 760 ng/kg and about 780 ng/kg, for a first administration time, where during the second phase the antibody is provided three times a week for a duration of one week in a dose amount of between about 760 ng/kg and about 780 ng/kg for a second administration time, and where during the third phase the antibody is provided once a week until remission in a dose amount of between about 2,200 ng/kg and 2,400 ng/kg.

In one embodiment, the method of treatment comprises a first phase and a second phase and a third phase, where during the first phase the antibody is provided three times a week for a duration of one week, where the first dose amount in the first phase is about 750 ng/kg, and the subsequent two dose amounts in the first phase are about 770 ng/kg, for a first administration time, where during the second phase the antibody is provided three times a week for a duration of one week in a dose amount about 770 ng/kg for a second administration time, and where during the third phase the antibody is provided once a week until remission in a dose amount of about 2,300 ng/kg.

In one embodiment, the method of treatment comprises a first phase and a second phase and a third phase, where during the first phase the antibody is provided three times a week for a duration of one week, where the first dose amount in the first phase is between about 700 ng/kg and about 800 ng/kg, and the subsequent two dose amounts in the first phase are between about 1,150 ng/kg and about 1,450 ng/kg, for a first administration time, where during the second phase the antibody is provided three times a week for a duration of one week in a dose amount of between about 1,150 ng/kg and about 1,450 ng/kg for a second administration time, and where during the third phase the antibody is provided once a week until remission in a dose amount of between about 3,000 ng/kg and 5,000 ng/kg.

In one embodiment, the method of treatment comprises a first phase and a second phase and a third phase, where during the first phase the antibody is provided three times a week for a duration of one week, where the first dose amount in the first phase is about 750 ng/kg, and the subsequent two dose amounts in the first phase are about 1,300 ng/kg, for a first administration time, where during the second phase the antibody is provided three times a week for a duration of one week in a dose amount about 1,300 ng/kg for a second administration time, and where during the third phase the antibody is provided once a week until remission in a dose amount of about 4,000 ng/kg.

In one embodiment, the method of treatment comprises a first phase and a second phase and a third phase, where during the first phase the antibody is provided three times a week for a duration of one week, where the first dose amount in the first phase is between about 700 ng/kg and about 800 ng/kg, and the subsequent two dose amounts in the first phase are between about 2,200 ng/kg and about 2,400 ng/kg, for a first administration time, where during the second phase the antibody is provided three times a week for a duration of one week in a dose amount of between about 2,200 ng/kg and about 2,400 ng/kg for a second administration time, and where during the third phase the antibody is provided once a week until remission in a dose amount of between about 6,000 ng/kg and 8,000 ng/kg.

In one embodiment, the method of treatment comprises a first phase and a second phase and a third phase, where during the first phase the antibody is provided three times a week for a duration of one week, where the first dose amount in the first phase is about 750 ng/kg and the subsequent two dose amounts in the first phase are about 2,300 ng/kg, for a first administration time, where during the second phase the antibody is provided three times a week for a duration of one week in a dose amount about 2,300 ng/kg for a second administration time, and where during the third phase the antibody is provided once a week until remission in a dose amount of about 7,000 ng/kg.

In one embodiment, the method of treatment comprises a first phase and a second phase and a third phase, where during the first phase the antibody is provided three times a week for a duration of one week, where the first dose amount in the first phase is between about 700 ng/kg and about 800 ng/kg, and the subsequent two dose amounts in the first phase are between about 3,000 ng/kg and about 5,000 ng/kg, for a first administration time, where during the second phase the antibody is provided three times a week for a duration of one week in a dose amount of between about 3,000 ng/kg and about 5,000 ng/kg for a second administration time, and where during the third phase the antibody is provided once a week until remission in a dose amount of between about 11,000 ng/kg and 13,000 ng/kg.

In one embodiment, the method of treatment comprises a first phase and a second phase and a third phase, where during the first phase the antibody is provided three times a week for a duration of one week, where the first dose amount in the first phase is about 750 ng/kg, and the subsequent two dose amounts in the first phase are about 4,000 ng/kg, for a first administration time, where during the second phase the antibody is provided three times a week for a duration of one week in a dose amount about 4,000 ng/kg for a second administration time, and where during the third phase the antibody is provided once a week until remission in a dose amount of about 12,000 ng/kg.

In one embodiment, the method of treatment comprises a first phase and a second phase and a third phase, where during the first phase the antibody is provided three times a week for a duration of one week, where the first dose amount in the first phase is between about 700 ng/kg and about 800 ng/kg, and the subsequent two dose amounts in the first phase are between about 6,000 ng/kg and about 7,000 ng/kg, for a first administration time, where during the second phase the antibody is provided three times a week for a duration of one week in a dose amount of between about 6,000 ng/kg and about 7,000 ng/kg for a second administration time, and where during the third phase the antibody is provided once a week until remission in a dose amount of between about 19,000 ng/kg and 21,000 ng/kg.

In one embodiment, the method of treatment comprises a first phase and a second phase and a third phase, where during the first phase the antibody is provided three times a week for a duration of one week, where the first dose amount in the first phase is about 750 ng/kg, and the subsequent two dose amounts in the first phase are about 6,700 ng/kg, for a first administration time, where during the second phase the antibody is provided three times a week for a duration of one week in a dose amount about 6,700 ng/kg for a second administration time, and where during the third phase the antibody is provided once a week until remission in a dose amount of about 20,000 ng/kg.

In one embodiment, the method of treatment comprises a first phase and a second phase and a third phase, where during the first phase the antibody is provided three times a week for a duration of one week, where the first dose amount in the first phase is between about 700 ng/kg and about 800 ng/kg, and the subsequent two dose amounts in the first phase are between about 11,000 ng/kg and about 13,000 ng/kg, for a first administration time, where during the second phase the antibody is provided three times a week for a duration of one week in a dose amount of between about 11,000 ng/kg and about 13,000 ng/kg for a second administration time, and where during the third phase the antibody is provided once a week until remission in a dose amount of between about 31,000 ng/kg and 38,000 ng/kg.

In one embodiment, the method of treatment comprises a first phase and a second phase and a third phase, where during the first phase the antibody is provided three times a week for a duration of one week, where the first dose amount in the first phase is about 750 ng/kg, and the subsequent two dose amounts in the first phase are about 11,700 ng/kg, for a first administration time, where during the second phase the antibody is provided three times a week for a duration of one week in a dose amount about 11,700 ng/kg for a second administration time, and where during the third phase the antibody is provided once a week until remission in a dose amount of about 35,000 ng/kg.

In one embodiment, the method of treatment comprises a first phase and a second phase and a third phase, where during the first phase, the bispecific anti-CD123×anti-CD3 antibody is administered to the human subject in a first dose amount found in Paragraph J, such as between about 600 ng/kg and about 900 ng/kg, such as about 750 ng/kg, where during the second phase, the bispecific anti-CD123×anti-CD3 antibody is administered to the human subject in a second dose amount of between about 120% and about 150% of the first dose amount, and where during the third phase, the bispecific anti-CD123×anti-CD3 antibody is administered to the human subject in a third dose amount of between about 120% and about 150% of the second dose amount, until remission.

In one embodiment, the method of treatment comprises a first phase and a second phase and a third phase and a fourth phase, where during the first phase, the bispecific anti-CD123×anti-CD3 antibody is administered to the human subject in a first dose amount found in Paragraph J, such as between about 600 ng/kg and about 900 ng/kg, such as about 750 ng/kg, where during the second phase, the bispecific anti-CD123×anti-CD3 antibody is administered to the human subject in a second dose amount of between about 120% and about 150% of the first dose amount, where during the third phase, the bispecific anti-CD123×anti-CD3 antibody is administered to the human subject in a third dose amount of between about 120% and about 150% of the second dose amount, and where during the fourth phase, the bispecific anti-CD123×anti-CD3 antibody is administered to the human subject in a fourth dose amount of between about 120% and about 150% of the third dose amount, until remission.

In one embodiment, the method of treatment comprises a first phase and a second phase and a third phase and a fourth phase and a fifth phase, where during the first phase, the bispecific anti-CD123×anti-CD3 antibody is administered to the human subject in a first dose amount found in Paragraph J, such as between about 600 ng/kg and about 900 ng/kg, such as about 750 ng/kg, where during the second phase, the bispecific anti-CD123×anti-CD3 antibody is administered to the human subject in a second dose amount of between about 120% and about 150% of the first dose amount, where during the third phase, the bispecific anti-CD123×anti-CD3 antibody is administered to the human subject in a third dose amount of between about 120% and about 150% of the second dose amount, where during the fourth phase, the bispecific anti-CD123×anti-CD3 antibody is administered to the human subject in a fourth dose amount of between about 120% and about 150% of the third dose amount, where during the fifth phase, the bispecific anti-CD123×anti-CD3 antibody is administered to the human subject in a fifth dose amount of between about 120% and about 150% of the fourth dose amount, until remission.

In one embodiment, the method of treatment comprises a first phase and a second phase and a third phase and a fourth phase and a fifth phase and a sixth phase, where during the first phase, the bispecific anti-CD123×anti-CD3 antibody is administered to the human subject in a first dose amount found in Paragraph J, such as between about 600 ng/kg and about 900 ng/kg, such as about 750 ng/kg, where during the second phase, the bispecific anti-CD123×anti-CD3 antibody is administered to the human subject in a second dose amount of between about 120% and about 150% of the first dose amount, where during the third phase, the bispecific anti-CD123×anti-CD3 antibody is administered to the human subject in a third dose amount of between about 120% and about 150% of the second dose amount, where during the fourth phase, the bispecific anti-CD123×anti-CD3 antibody is administered to the human subject in a fourth dose amount of between about 120% and about 150% of the third dose amount, where during the fifth phase, the bispecific anti-CD123×anti-CD3 antibody is administered to the human subject in a fifth dose amount of between about 120% and about 150% of the fourth dose amount, and where during the sixth phase, the bispecific anti-CD123×anti-CD3 antibody is administered to the human subject in a sixth dose amount of between about 120% and about 150% of the fifth dose amount, until remission.

In one embodiment, the method of treatment comprises a first phase and a second phase and a third phase and a fourth phase and a fifth phase and a sixth phase and a seventh phase, where during the first phase, the bispecific anti-CD123×anti-CD3 antibody is administered to the human subject in a first dose amount found in Paragraph J, such as between about 600 ng/kg and about 900 ng/kg, such as about 750 ng/kg, where during the second phase, the bispecific anti-CD123×anti-CD3 antibody is administered to the human subject in a second dose amount of between about 120% and about 150% of the first dose amount, where during the third phase, the bispecific anti-CD123×anti-CD3 antibody is administered to the human subject in a third dose amount of between about 120% and about 150% of the second dose amount, where during the fourth phase, the bispecific anti-CD123×anti-CD3 antibody is administered to the human subject in a fourth dose amount of between about 120% and about 150% of the third dose amount, where during the fifth phase, the bispecific anti-CD123×anti-CD3 antibody is administered to the human subject in a fifth dose amount of between about 120% and about 150% of the fourth dose amount, where during the sixth phase, the bispecific anti-CD123×anti-CD3 antibody is administered to the human subject in a sixth dose amount of between about 120% and about 150% of the fifth dose amount, and where during the seventh phase, the bispecific anti-CD123×anti-CD3 antibody is administered to the human subject in a seventh dose amount of between about 120% and about 150% of the sixth dose amount, until remission.

In one embodiment, the method of treatment comprises a first phase and a second phase and a third phase, where during the first phase, the bispecific anti-CD123×anti-CD3 antibody is administered to the human subject once a week for one week in a first dose amount found in Paragraph J, such as between about 600 ng/kg and about 900 ng/kg, such as about 750 ng/kg, where during the second phase, the bispecific anti-CD123×anti-CD3 antibody is administered to the human subject once a week for one week in a second dose amount of between about 120% and about 150% of the first dose amount, and where during the third phase, the bispecific anti-CD123×anti-CD3 antibody is administered to the human subject once a week until remission, in a third dose amount of between about 120% and about 150% of the second dose amount.

In one embodiment, the method of treatment comprises a first phase and a second phase and a third phase, where during the first phase, the bispecific anti-CD123×anti-CD3 antibody is administered to the human subject three times a week for one week, where the first dose amount in the first phase is found in Paragraph J, such as between about 600 ng/kg and about 900 ng/kg, such as about 750 ng/kg, and the second dose amount and third dose amount in the first phase are each between about 120% and about 150% of the first dose amount in the first phase, where during the second phase, the bispecific anti-CD123×anti-CD3 antibody is administered to the human subject once a week for one week in a second dose amount of between about 120% and about 150% of the third dose amount of the first phase, and where during the third phase, the bispecific anti-CD123×anti-CD3 antibody is administered to the human subject once a week until remission, in a third dose amount of between about 120% and about 150% of the second dose amount.

In one embodiment, the method of treatment comprises a first phase and a second phase and a third phase, where during the first phase, the bispecific anti-CD123×anti-CD3 antibody is administered to the human subject three times a week for one week, where the first dose amount in the first phase is found in Paragraph J, such as between about 600 ng/kg and about 900 ng/kg, such as about 750 ng/kg, and the second dose amount in the first phase is between about 120% and about 150% of the first dose amount in the first phase, and the third dose amount in the first phase is between about 120% and about 150% of the second dose amount in the first phase, where during the second phase, the bispecific anti-CD123×anti-CD3 antibody is administered to the human subject once a week for one week in a second dose amount of between about 120% and about 150% of the third dose amount of the first phase, and where during the third phase, the bispecific anti-CD123×anti-CD3 antibody is administered to the human subject once a week until remission, in a third dose amount of between about 120% and about 150% of the second dose amount.

In one embodiment, the method of treatment comprises a first phase and a second phase and a third phase, where during the first phase, the bispecific anti-CD123×anti-CD3 antibody is administered to the human subject three times a week for one week, where the first dose amount in the first phase is about 750 ng/kg, and the second dose amount in the first phase is between about 1,100 ng/kg and about 1,200 ng/kg, and the third dose amount in the first phase is between about 1,700 ng/kg and about 1,800 ng/kg, where during the second phase, the bispecific anti-CD123×anti-CD3 antibody is administered to the human subject once a week for one week in a second dose amount of between about 120% and about 150% of the third dose amount of the first phase, and where during the third phase, the bispecific anti-CD123×anti-CD3 antibody is administered to the human subject once a week until remission, in a third dose amount of between about 120% and about 150% of the second dose amount.

In one embodiment, the method of treatment comprises a first phase and a second phase and a third phase, where during the first phase, the bispecific anti-CD123×anti-CD3 antibody is administered to the human subject three times a week for one week, where the first dose amount in the first phase is about 750 ng/kg, and the second dose amount in the first phase is between about 1,100 ng/kg and about 1,200 ng/kg, and the third dose amount in the first phase is between about 1,700 ng/kg and about 1,800 ng/kg, where during the second phase, the bispecific anti-CD123×anti-CD3 antibody is administered to the human subject once a week for one week in a second dose amount of between about 2,500 ng/kg and about 2,700 ng/kg, and where during the third phase, the bispecific anti-CD123×anti-CD3 antibody is administered to the human subject once a week until remission, in a third dose amount of between about 3,750 ng/kg and about 4,250 ng/kg.

In one embodiment, the method of treatment comprises a first phase and a second phase and a third phase and a fourth phase, where during the first phase, the bispecific anti-CD123×anti-CD3 antibody is administered to the human subject once a week for one week in a first dose amount found in Paragraph J, such as between about 600 ng/kg and about 900 ng/kg, such as about 750 ng/kg, where during the second phase, the bispecific anti-CD123×anti-CD3 antibody is administered to the human subject once a week for one week in a second dose amount of between about 120% and about 150% of the first dose amount, where during the third phase, the bispecific anti-CD123×anti-CD3 antibody is administered to the human subject once a week for one week, in a third dose amount of between about 120% and about 150% of the second dose amount, and where during the fourth phase, the bispecific anti-CD123×anti-CD3 antibody is administered to the human subject once a week until remission, in a fourth dose amount of between about 120% and about 150% of the third dose amount.

In one embodiment, the method of treatment comprises a first phase and a second phase and a third phase and a fourth phase, where during the first phase, the bispecific anti-CD123×anti-CD3 antibody is administered to the human subject three times a week for one week, where the first dose amount in the first phase is found in Paragraph J, such as between about 600 ng/kg and about 900 ng/kg, such as about 750 ng/kg, and the second dose amount and third dose amount in the first phase are each between about 120% and about 150% of the first dose amount in the first phase, where during the second phase, the bispecific anti-CD123×anti-CD3 antibody is administered to the human subject once a week for one week in a second dose amount of between about 120% and about 150% of the third dose amount of the first phase, where during the third phase, the bispecific anti-CD123×anti-CD3 antibody is administered to the human subject once a week for one week, in a third dose amount of between about 120% and about 150% of the second dose amount, and where during the fourth phase, the bispecific anti-CD123×anti-CD3 antibody is administered to the human subject once a week until remission, in a fourth dose amount of between about 120% and about 150% of the third dose amount.

In one embodiment, the method of treatment comprises a first phase and a second phase and a third phase and a fourth phase, where during the first phase, the bispecific anti-CD123×anti-CD3 antibody is administered to the human subject three times a week for one week, where the first dose amount in the first phase is found in Paragraph J, such as between about 600 ng/kg and about 900 ng/kg, such as about 750 ng/kg, and the second dose amount in the first phase is between about 120% and about 150% of the first dose amount in the first phase, and the third dose amount in the first phase is between about 120% and about 150% of the second dose amount in the first phase, where during the second phase, the bispecific anti-CD123×anti-CD3 antibody is administered to the human subject once a week for one week in a second dose amount of between about 120% and about 150% of the third dose amount of the first phase, where during the third phase, the bispecific anti-CD123×anti-CD3 antibody is administered to the human subject once a week for one week, in a third dose amount of between about 120% and about 150% of the second dose amount, and where during the fourth phase, the bispecific anti-CD123×anti-CD3 antibody is administered to the human subject once a week until remission, in a fourth dose amount of between about 120% and about 150% of the third dose amount.

In one embodiment, the method of treatment comprises a first phase and a second phase and a third phase and a fourth phase, where during the first phase, the bispecific anti-CD123×anti-CD3 antibody is administered to the human subject three times a week for one week, where the first dose amount in the first phase is about 750 ng/kg, and the second dose amount in the first phase is between about 1,100 ng/kg and about 1,200 ng/kg, and the third dose amount in the first phase is between about 1,700 ng/kg and about 1,800 ng/kg, where during the second phase, the bispecific anti-CD123×anti-CD3 antibody is administered to the human subject once a week for one week in a second dose amount of between about 120% and about 150% of the third dose amount of the first phase, where during the third phase, the bispecific anti-CD123×anti-CD3 antibody is administered to the human subject once a week for one week, in a third dose amount of between about 120% and about 150% of the second dose amount and where during the fourth phase, the bispecific anti-CD123×anti-CD3 antibody is administered to the human subject once a week until remission, in a fourth dose amount of between about 120% and about 150% of the third dose amount.

In one embodiment, the method of treatment comprises a first phase and a second phase and a third phase and a fourth phase, where during the first phase, the bispecific anti-CD123×anti-CD3 antibody is administered to the human subject three times a week for one week, where the first dose amount in the first phase is about 750 ng/kg, and the second dose amount in the first phase is between about 1,100 ng/kg and about 1,200 ng/kg, and the third dose amount in the first phase is between about 1,700 ng/kg and about 1,800 ng/kg, where during the second phase, the bispecific anti-CD123×anti-CD3 antibody is administered to the human subject once a week for one week in a second dose amount of between about 2,500 ng/kg and about 2,700 ng/kg, and where during the third phase, the bispecific anti-CD123×anti-CD3 antibody is administered to the human subject once a week for one week, in a third dose amount of between about 3,750 ng/kg and about 4,250 ng/kg, and where during the fourth phase, the bispecific anti-CD123×anti-CD3 antibody is administered to the human subject once a week until remission, in a fourth dose amount of between about 5,000 ng/kg and about 7,000 ng/kg.

In one embodiment, the method of treatment comprises a first phase and a second phase and a third phase and a fourth phase and a fifth phase, where during the first phase, the bispecific anti-CD123×anti-CD3 antibody is administered to the human subject once a week for one week in a first dose amount found in Paragraph J, such as between about 600 ng/kg and about 900 ng/kg, such as about 750 ng/kg, where during the second phase, the bispecific anti-CD123×anti-CD3 antibody is administered to the human subject once a week for one week in a second dose amount of between about 120% and about 150% of the first dose amount, where during the third phase, the bispecific anti-CD123×anti-CD3 antibody is administered to the human subject once a week for one week, in a third dose amount of between about 120% and about 150% of the second dose amount, where during the fourth phase, the bispecific anti-CD123×anti-CD3 antibody is administered to the human subject once a week for one week, in a fourth dose amount of between about 120% and about 150% of the third dose amount, and where during the fifth phase, the bispecific anti-CD123×anti-CD3 antibody is administered to the human subject once a week until remission, in a fifth dose amount of between about 120% and about 150% of the fourth dose amount.

In one embodiment, the method of treatment comprises a first phase and a second phase and a third phase and a fourth phase and a fifth phase, where during the first phase, the bispecific anti-CD123×anti-CD3 antibody is administered to the human subject three times a week for one week, where the first dose amount in the first phase is found in Paragraph J, such as between about 600 ng/kg and about 900 ng/kg, such as about 750 ng/kg, and the second dose amount and third dose amount in the first phase are each between about 120% and about 150% of the first dose amount in the first phase, where during the second phase, the bispecific anti-CD123×anti-CD3 antibody is administered to the human subject once a week for one week in a second dose amount of between about 120% and about 150% of the third dose amount of the first phase, where during the third phase, the bispecific anti-CD123×anti-CD3 antibody is administered to the human subject once a week for one week, in a third dose amount of between about 120% and about 150% of the second dose amount, where during the fourth phase, the bispecific anti-CD123×anti-CD3 antibody is administered to the human subject once a week for one week, in a fourth dose amount of between about 120% and about 150% of the third dose amount, and where during the fifth phase, the bispecific anti-CD123×anti-CD3 antibody is administered to the human subject once a week until remission, in a fifth dose amount of between about 120% and about 150% of the fourth dose amount.

In one embodiment, the method of treatment comprises a first phase and a second phase and a third phase and a fourth phase and a fifth phase, where during the first phase, the bispecific anti-CD123×anti-CD3 antibody is administered to the human subject three times a week for one week, where the first dose amount in the first phase is found in Paragraph J, such as between about 600 ng/kg and about 900 ng/kg, such as about 750 ng/kg, and the second dose amount in the first phase is between about 120% and about 150% of the first dose amount in the first phase, and the third dose amount in the first phase is between about 120% and about 150% of the second dose amount in the first phase, where during the second phase, the bispecific anti-CD123×anti-CD3 antibody is administered to the human subject once a week for one week in a second dose amount of between about 120% and about 150% of the third dose amount of the first phase, where during the third phase, the bispecific anti-CD123×anti-CD3 antibody is administered to the human subject once a week for one week, in a third dose amount of between about 120% and about 150% of the second dose amount, where during the fourth phase, the bispecific anti-CD123×anti-CD3 antibody is administered to the human subject once a week for one week, in a fourth dose amount of between about 120% and about 150% of the third dose amount, and where during the fifth phase, the bispecific anti-CD123×anti-CD3 antibody is administered to the human subject once a week until remission, in a fifth dose amount of between about 120% and about 150% of the fourth dose amount.

In one embodiment, the method of treatment comprises a first phase and a second phase and a third phase and a fourth phase and a fifth phase, where during the first phase, the bispecific anti-CD123×anti-CD3 antibody is administered to the human subject three times a week for one week, where the first dose amount in the first phase is about 750 ng/kg, and the second dose amount in the first phase is between about 1,100 ng/kg and about 1,200 ng/kg, and the third dose amount in the first phase is between about 1,700 ng/kg and about 1,800 ng/kg, where during the second phase, the bispecific anti-CD123×anti-CD3 antibody is administered to the human subject once a week for one week in a second dose amount of between about 120% and about 150% of the third dose amount of the first phase, where during the third phase, the bispecific anti-CD123×anti-CD3 antibody is administered to the human subject once a week for one week, in a third dose amount of between about 120% and about 150% of the second dose amount and where during the fourth phase, the bispecific anti-CD123×anti-CD3 antibody is administered to the human subject once a week for one week, in a fourth dose amount of between about 120% and about 150% of the third dose amount, and where during the fifth phase, the bispecific anti-CD123×anti-CD3 antibody is administered to the human subject once a week until remission, in a fifth dose amount of between about 120% and about 150% of the fourth dose amount.

In one embodiment, the method of treatment comprises a first phase and a second phase and a third phase and a fourth phase and a fifth phase, where during the first phase, the bispecific anti-CD123×anti-CD3 antibody is administered to the human subject three times a week for one week, where the first dose amount in the first phase is about 750 ng/kg, and the second dose amount in the first phase is between about 1,100 ng/kg and about 1,200 ng/kg, and the third dose amount in the first phase is between about 1,700 ng/kg and about 1,800 ng/kg, where during the second phase, the bispecific anti-CD123×anti-CD3 antibody is administered to the human subject once a week for one week in a second dose amount of between about 2,500 ng/kg and about 2,700 ng/kg, and where during the third phase, the bispecific anti-CD123×anti-CD3 antibody is administered to the human subject once a week for one week, in a third dose amount of between about 3,750 ng/kg and about 4,250 ng/kg, and where during the fourth phase, the bispecific anti-CD123×anti-CD3 antibody is administered to the human subject once a week for one week, in a fourth dose amount of between about 5,000 ng/kg and about 7,000 ng/kg and where during the fifth phase, the bispecific anti-CD123×anti-CD3 antibody is administered to the human subject once a week until remission, in a fifth dose amount of between about 8,000 ng/kg and about 10,000 ng/kg.

In one embodiment, the method of treatment comprises a first phase and a second phase and a third phase and a fourth phase and a fifth phase and a sixth phase, where during the first phase, the bispecific anti-CD123×anti-CD3 antibody is administered to the human subject once a week for one week in a first dose amount found in Paragraph J, such as between about 600 ng/kg and about 900 ng/kg, such as about 750 ng/kg, where during the second phase, the bispecific anti-CD123×anti-CD3 antibody is administered to the human subject once a week for one week in a second dose amount of between about 120% and about 150% of the first dose amount, where during the third phase, the bispecific anti-CD123×anti-CD3 antibody is administered to the human subject once a week for one week, in a third dose amount of between about 120% and about 150% of the second dose amount, where during the fourth phase, the bispecific anti-CD123×anti-CD3 antibody is administered to the human subject once a week for one week, in a fourth dose amount of between about 120% and about 150% of the third dose amount, where during the fifth phase, the bispecific anti-CD123×anti-CD3 antibody is administered to the human subject once a week for one week, in a fifth dose amount of between about 120% and about 150% of the fourth dose amount, and where during the sixth phase, the bispecific anti-CD123×anti-CD3 antibody is administered to the human subject once a week until remission, in a sixth dose amount of between about 120% and about 150% of the fifth dose amount.

In one embodiment, the method of treatment comprises a first phase and a second phase and a third phase and a fourth phase and a fifth phase and a sixth phase, where during the first phase, the bispecific anti-CD123×anti-CD3 antibody is administered to the human subject three times a week for one week, where the first dose amount in the first phase is found in Paragraph J, such as between about 600 ng/kg and about 900 ng/kg, such as about 750 ng/kg, and the second dose amount and third dose amount in the first phase are each between about 120% and about 150% of the first dose amount in the first phase, where during the second phase, the bispecific anti-CD123×anti-CD3 antibody is administered to the human subject once a week for one week in a second dose amount of between about 120% and about 150% of the third dose amount of the first phase, where during the third phase, the bispecific anti-CD123×anti-CD3 antibody is administered to the human subject once a week for one week, in a third dose amount of between about 120% and about 150% of the second dose amount, where during the fourth phase, the bispecific anti-CD123×anti-CD3 antibody is administered to the human subject once a week for one week, in a fourth dose amount of between about 120% and about 150% of the third dose amount, where during the fifth phase, the bispecific anti-CD123×anti-CD3 antibody is administered to the human subject once a week for one week, in a fifth dose amount of between about 120% and about 150% of the fourth dose amount, and where during the sixth phase, the bispecific anti-CD123×anti-CD3 antibody is administered to the human subject once a week until remission, in a sixth dose amount of between about 120% and about 150% of the fifth dose amount.

In one embodiment, the method of treatment comprises a first phase and a second phase and a third phase and a fourth phase and a fifth phase and a sixth phase, where during the first phase, the bispecific anti-CD123×anti-CD3 antibody is administered to the human subject three times a week for one week, where the first dose amount in the first phase is found in Paragraph J, such as between about 600 ng/kg and about 900 ng/kg, such as about 750 ng/kg, and the second dose amount in the first phase is between about 120% and about 150% of the first dose amount in the first phase, and the third dose amount in the first phase is between about 120% and about 150% of the second dose amount in the first phase, where during the second phase, the bispecific anti-CD123×anti-CD3 antibody is administered to the human subject once a week for one week in a second dose amount of between about 120% and about 150% of the third dose amount of the first phase, where during the third phase, the bispecific anti-CD123×anti-CD3 antibody is administered to the human subject once a week for one week, in a third dose amount of between about 120% and about 150% of the second dose amount, where during the fourth phase, the bispecific anti-CD123×anti-CD3 antibody is administered to the human subject once a week for one week, in a fourth dose amount of between about 120% and about 150% of the third dose amount, where during the fifth phase, the bispecific anti-CD123×anti-CD3 antibody is administered to the human subject once a week for one week, in a fifth dose amount of between about 120% and about 150% of the fourth dose amount, and where during the sixth phase, the bispecific anti-CD123×anti-CD3 antibody is administered to the human subject once a week until remission, in a sixth dose amount of between about 120% and about 150% of the fifth dose amount.

In one embodiment, the method of treatment comprises a first phase and a second phase and a third phase and a fourth phase and a fifth phase and a sixth phase, where during the first phase, the bispecific anti-CD123×anti-CD3 antibody is administered to the human subject three times a week for one week, where the first dose amount in the first phase is about 750 ng/kg, and the second dose amount in the first phase is between about 1,100 ng/kg and about 1,200 ng/kg, and the third dose amount in the first phase is between about 1,700 ng/kg and about 1,800 ng/kg, where during the second phase, the bispecific anti-CD123×anti-CD3 antibody is administered to the human subject once a week for one week in a second dose amount of between about 120% and about 150% of the third dose amount of the first phase, where during the third phase, the bispecific anti-CD123×anti-CD3 antibody is administered to the human subject once a week for one week, in a third dose amount of between about 120% and about 150% of the second dose amount, where during the fourth phase, the bispecific anti-CD123×anti-CD3 antibody is administered to the human subject once a week for one week, in a fourth dose amount of between about 120% and about 150% of the third dose amount, and where during the fifth phase, the bispecific anti-CD123×anti-CD3 antibody is administered to the human subject once a week for one week, in a fifth dose amount of between about 120% and about 150% of the fourth dose amount, and where during the sixth phase, the bispecific anti-CD123×anti-CD3 antibody is administered to the human subject once a week until remission, in a sixth dose amount of between about 120% and about 150% of the fifth dose amount.

In one embodiment, the method of treatment comprises a first phase and a second phase and a third phase and a fourth phase and a fifth phase and a sixth phase, where during the first phase, the bispecific anti-CD123×anti-CD3 antibody is administered to the human subject three times a week for one week, where the first dose amount in the first phase is about 750 ng/kg, and the second dose amount in the first phase is between about 1,100 ng/kg and about 1,200 ng/kg, and the third dose amount in the first phase is between about 1,700 ng/kg and about 1,800 ng/kg, where during the second phase, the bispecific anti-CD123×anti-CD3 antibody is administered to the human subject once a week for one week in a second dose amount of between about 2,500 ng/kg and about 2,700 ng/kg, and where during the third phase, the bispecific anti-CD123×anti-CD3 antibody is administered to the human subject once a week for one week, in a third dose amount of between about 3,750 ng/kg and about 4,250 ng/kg and where during the fourth phase, the bispecific anti-CD123×anti-CD3 antibody is administered to the human subject once a week for one week, in a fourth dose amount of between about 5,000 ng/kg and about 7,000 ng/kg and where during the fifth phase, the bispecific anti-CD123×anti-CD3 antibody is administered to the human subject once a week for one week, in a fifth dose amount of between about 8,000 ng/kg and about 10,000 ng/kg, and where during the sixth phase, the bispecific anti-CD123×anti-CD3 antibody is administered to the human subject once a week until remission, in a sixth dose amount of between about 11,000 ng/kg and about 13,000 ng/kg.

In one embodiment, the method of treatment comprises a first phase and a second phase and a third phase and a fourth phase and a fifth phase and a sixth phase and a seventh phase, where during the first phase, the bispecific anti-CD123×anti-CD3 antibody is administered to the human subject once a week for one week in a first dose amount found in Paragraph J, such as between about 600 ng/kg and about 900 ng/kg, such as about 750 ng/kg, where during the second phase, the bispecific anti-CD123×anti-CD3 antibody is administered to the human subject once a week for one week in a second dose amount of between about 120% and about 150% of the first dose amount, where during the third phase, the bispecific anti-CD123×anti-CD3 antibody is administered to the human subject once a week for one week, in a third dose amount of between about 120% and about 150% of the second dose amount, where during the fourth phase, the bispecific anti-CD123×anti-CD3 antibody is administered to the human subject once a week for one week, in a fourth dose amount of between about 120% and about 150% of the third dose amount, where during the fifth phase, the bispecific anti-CD123×anti-CD3 antibody is administered to the human subject once a week for one week, in a fifth dose amount of between about 120% and about 150% of the fourth dose amount, where during the sixth phase, the bispecific anti-CD123×anti-CD3 antibody is administered to the human subject once a week for one week, in a sixth dose amount of between about 120% and about 150% of the fifth dose amount, and where during the seventh phase, the bispecific anti-CD123×anti-CD3 antibody is administered to the human subject once a week until remission, in a seventh dose amount of between about 120% and about 150% of the sixth dose amount.

In one embodiment, the method of treatment comprises a first phase and a second phase and a third phase and a fourth phase and a fifth phase and a sixth phase and a seventh phase, where during the first phase, the bispecific anti-CD123×anti-CD3 antibody is administered to the human subject three times a week for one week, where the first dose amount in the first phase is found in Paragraph J, such as between about 600 ng/kg and about 900 ng/kg, such as about 750 ng/kg, and the second dose amount and third dose amount in the first phase are each between about 120% and about 150% of the first dose amount in the first phase, where during the second phase, the bispecific anti-CD123×anti-CD3 antibody is administered to the human subject once a week for one week in a second dose amount of between about 120% and about 150% of the third dose amount of the first phase, where during the third phase, the bispecific anti-CD123×anti-CD3 antibody is administered to the human subject once a week for one week, in a third dose amount of between about 120% and about 150% of the second dose amount, where during the fourth phase, the bispecific anti-CD123×anti-CD3 antibody is administered to the human subject once a week for one week, in a fourth dose amount of between about 120% and about 150% of the third dose amount, where during the fifth phase, the bispecific anti-CD123×anti-CD3 antibody is administered to the human subject once a week for one week, in a fifth dose amount of between about 120% and about 150% of the fourth dose amount, where during the sixth phase, the bispecific anti-CD123×anti-CD3 antibody is administered to the human subject once a week for one week, in a sixth dose amount of between about 120% and about 150% of the fifth dose amount, and where during the seventh phase, the bispecific anti-CD123×anti-CD3 antibody is administered to the human subject once a week until remission, in a seventh dose amount of between about 120% and about 150% of the sixth dose amount.

In one embodiment, the method of treatment comprises a first phase and a second phase and a third phase and a fourth phase and a fifth phase and a sixth phase and a seventh phase, where during the first phase, the bispecific anti-CD123×anti-CD3 antibody is administered to the human subject three times a week for one week, where the first dose amount in the first phase is found in Paragraph J, such as between about 600 ng/kg and about 900 ng/kg, such as about 750 ng/kg, and the second dose amount in the first phase is between about 120% and about 150% of the first dose amount in the first phase, and the third dose amount in the first phase is between about 120% and about 150% of the second dose amount in the first phase, where during the second phase, the bispecific anti-CD123×anti-CD3 antibody is administered to the human subject once a week for one week in a second dose amount of between about 120% and about 150% of the third dose amount of the first phase, where during the third phase, the bispecific anti-CD123×anti-CD3 antibody is administered to the human subject once a week for one week, in a third dose amount of between about 120% and about 150% of the second dose amount, where during the fourth phase, the bispecific anti-CD123×anti-CD3 antibody is administered to the human subject once a week for one week, in a fourth dose amount of between about 120% and about 150% of the third dose amount, where during the fifth phase, the bispecific anti-CD123×anti-CD3 antibody is administered to the human subject once a week for one week, in a fifth dose amount of between about 120% and about 150% of the fourth dose amount, where during the sixth phase, the bispecific anti-CD123×anti-CD3 antibody is administered to the human subject once a week for one week, in a sixth dose amount of between about 120% and about 150% of the fifth dose amount, and where during the seventh phase, the bispecific anti-CD123×anti-CD3 antibody is administered to the human subject once a week until remission, in a seventh dose amount of between about 120% and about 150% of the sixth dose amount.

In one embodiment, the method of treatment comprises a first phase and a second phase and a third phase, where during the first phase, the bispecific anti-CD123×anti-CD3 antibody is administered to the human subject three times a week for one week, where the first dose amount in the first phase is found in Paragraph J, such as between about 600 ng/kg and about 900 ng/kg, such as about 750 ng/kg, and the second dose amount and third dose amount in the first phase are each between about 120% and about 150% of the first dose amount in the first phase, where during the second phase, the bispecific anti-CD123×anti-CD3 antibody is administered to the human subject three times a week for one week, where the first dose amount in the second phase is between about 120% and about 150% of the third dose amount in the first phase, and the second dose amount and third dose amount in the second phase are each between about 120% and about 150% of the first dose amount in the second phase, and where during the third phase, the bispecific anti-CD123×anti-CD3 antibody is administered to the human subject once a week until remission, in a third dose amount of between about 120% and about 150% of the third dose amount in the second phase.

In one embodiment, the method of treatment comprises a first phase and a second phase and a third phase, where during the first phase, the bispecific anti-CD123×anti-CD3 antibody is administered to the human subject three times a week for one week, where the first dose amount in the first phase is found in Paragraph J, such as between about 600 ng/kg and about 900 ng/kg, such as about 750 ng/kg, and the second dose amount in the first phase is between about 120% and about 150% of the first dose amount in the first phase, and the third dose amount in the first phase is between about 120% and about 150% of the second dose amount in the first phase, where during the second phase, the bispecific anti-CD123×anti-CD3 antibody is administered to the human subject three times a week for one week, where the first dose amount in the second phase is between about 120% and about 150% of the third dose amount in the first phase, and the second dose amount in the second phase is between about 120% and about 150% of the first dose amount in the second phase, and the third dose amount in the second phase is between about 120% and about 150% of the second dose amount in the second phase, and where during the third phase, the bispecific anti-CD123×anti-CD3 antibody is administered to the human subject once a week until remission, in a third dose amount of between about 120% and about 150% of the third dose amount in the second phase.

In one embodiment, the method of treatment comprises a first phase and a second phase and a third phase, where during the first phase, the bispecific anti-CD123×anti-CD3 antibody is administered to the human subject three times a week for one week, where the first dose amount in the first phase is about 750 ng/kg, and the second dose amount in the first phase is between about 1,100 ng/kg and about 1,200 ng/kg, and the third dose amount in the first phase is between about 1,700 ng/kg and about 1,800 ng/kg, where during the second phase, the bispecific anti-CD123×anti-CD3 antibody is administered to the human subject three times a week for one week, where the second dose amount in the second phase is between about 1,000 ng/kg and about 1,400 ng/kg, and where during the third phase, the bispecific anti-CD123×anti-CD3 antibody is administered to the human subject once a week until remission, in a third dose amount of between about 3,750 ng/kg and about 4,250 ng/kg.

In one embodiment, the method of treatment comprises a first phase and a second phase and a third phase, where during the first phase, the bispecific anti-CD123×anti-CD3 antibody is administered to the human subject three times a week for one week, where the first dose amount in the first phase is about 750 ng/kg, and the second dose amount in the first phase is between about 1,100 ng/kg and about 1,200 ng/kg, and the third dose amount in the first phase is between about 1,700 ng/kg and about 1,800 ng/kg, where during the second phase, the bispecific anti-CD123×anti-CD3 antibody is administered to the human subject three times a week for one week, where the second dose amount in the second phase is between about 2,000 ng/kg and about 2,500 ng/kg, and where during the third phase, the bispecific anti-CD123×anti-CD3 antibody is administered to the human subject once a week until remission, in a third dose amount of between about 6,000 ng/kg and about 8,000 ng/kg.

In one embodiment, the method of treatment comprises a first phase and a second phase and a third phase, where during the first phase, the bispecific anti-CD123×anti-CD3 antibody is administered to the human subject three times a week for one week, where the first dose amount in the first phase is about 750 ng/kg, and the second dose amount in the first phase is between about 1,100 ng/kg and about 1,200 ng/kg, and the third dose amount in the first phase is between about 1,700 ng/kg and about 1,800 ng/kg, where during the second phase, the bispecific anti-CD123×anti-CD3 antibody is administered to the human subject three times a week for one week, where the second dose amount in the second phase is between about 3,750 ng/kg and about 4,250 ng/kg, and where during the third phase, the bispecific anti-CD123×anti-CD3 antibody is administered to the human subject once a week until remission, in a third dose amount of between about 11,000 ng/kg and about 13,000 ng/kg.

In one embodiment, the method of treatment comprises a first phase and a second phase and a third phase, where during the first phase, the bispecific anti-CD123×anti-CD3 antibody is administered to the human subject three times a week for one week, where the first dose amount in the first phase is about 750 ng/kg, and the second dose amount in the first phase is between about 1,100 ng/kg and about 1,200 ng/kg, and the third dose amount in the first phase is between about 1,700 ng/kg and about 1,800 ng/kg, where during the second phase, the bispecific anti-CD123×anti-CD3 antibody is administered to the human subject three times a week for one week, where the first dose amount in the second phase is between about 2,000 ng/kg and about 2,500 ng/kg, and the second dose amount in the second phase is between about 3,500 ng/kg and about 4,500 ng/kg, and the third dose amount in the second phase is between about 5,500 ng/kg and about 6,500 ng/kg, and where during the third phase, the bispecific anti-CD123×anti-CD3 antibody is administered to the human subject once a week until remission, in a third dose amount of between about 11,000 ng/kg and about 13,000 ng/kg.

In one embodiment, the method of treatment comprises a first phase and a second phase and a third phase, where during the first phase, the bispecific anti-CD123×anti-CD3 antibody is administered to the human subject three times a week for one week, where the first dose amount in the first phase is about 750 ng/kg, and the second dose amount in the first phase is between about 1,100 ng/kg and about 1,200 ng/kg, and the third dose amount in the first phase is between about 1,700 ng/kg and about 1,800 ng/kg, where during the second phase, the bispecific anti-CD123×anti-CD3 antibody is administered to the human subject three times a week for one week, where the first dose amount in the second phase is between about 2,000 ng/kg and about 2,500 ng/kg, and the second dose amount in the second phase is between about 3,500 ng/kg and about 4,500 ng/kg, and the third dose amount in the second phase is between about 5,500 ng/kg and about 6,500 ng/kg, and where during the third phase, the bispecific anti-CD123×anti-CD3 antibody is administered to the human subject once a week until remission, in a third dose amount of between about 19,000 ng/kg and about 21,000 ng/kg.

In some embodiments, the antibody comprises a bispecific anti-CD123×anti-CD3 antibody (e.g., XmAb14045) that is administered intravenously. In some embodiments, the bispecific anti-CD123×anti-CD3 antibody is administered via continuous infusion. In some embodiments, the bispecific anti-CD123×anti-CD3 antibody is administered intravenously, continuous infusion, or both. Should there be more than two treatments, any combination of intravenous administration or continuous infusion can be used. In some embodiments, the bispecific anti-CD123×anti-CD3 antibody (e.g., XmAb14045) is administered until non-efficacy is determined, an unacceptable level of toxicity is observed, or is voluntary terminated by the human subject.

In some embodiments, the bispecific anti-CD123×anti-CD3 antibody (e.g., XmAb14045) is a front line therapy, second line therapy, third line therapy, fourth line therapy, fifth line therapy, or sixth line therapy.

In some embodiments, the bispecific anti-CD123×anti-CD3 antibody (e.g., XmAb14045) treats a refractory leukemia. In some embodiments, the bispecific anti-CD123×anti-CD3 antibody (e.g., XmAb14045) is a maintenance therapy. In one embodiment, for any method described herein, when the CD123-expressing cancer is in remission, such as partial remission and/or complete remission, the method further includes providing the bispecific anti-CD123×anti-CD3 antibody (e.g., XmAb14045) according to an every other week dosage regimen described herein, at the same dose amount for remission, such as partial remission and/or complete remission, until non-efficacy is determined, an unacceptable level of toxicity is observed, or is voluntary terminated by the human subject. In one embodiment, for any method described herein, when the CD123-expressing cancer is in remission, such as partial remission and/or complete remission, the method further includes providing the bispecific anti-CD123×anti-CD3 antibody (e.g., XmAb14045) according to a once a week dosage regimen described herein or a once every three weeks dosage regimen described herein or a once every four weeks dosage regimen described herein or a two times a month dosage regimen described herein or a three times a month dosage regimen described herein or a monthly dosage regimen described herein, at the same dose amount for remission, such as partial remission and/or complete remission, or within about 10% of the dose amount (plus or minus), or within about 20% of the dose amount (plus or minus), of within about 30% of the dose amount (plus or minus), until non-efficacy is determined, an unacceptable level of toxicity is observed, or is voluntary terminated by the human subject.

A medical professional can readily determine and prescribe the effective amount of the antibody composition required. For example, a physician could start doses of the medicament employed in the antibody composition at levels lower than that required in order to achieve the desired therapeutic effect and gradually increase the dosage until the desired effect is achieved.

### Combination Therapy

In certain instances, a bispecific anti-CD123×anti-CD3 antibody (e.g., XmAb14045) described herein can be used in combination with another therapeutic agent. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces one or more symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The bispecific anti-CD123×anti-CD3 antibody (e.g., XmAb14045) and at least one additional therapeutic agent can be administered simultaneously, in the same or in separate compositions, or sequentially. For sequential administration, the bispecific anti-CD123×anti-CD3 antibody (e.g., XmAb14045) described herein can be administered first, and the additional agent can be administered second, or vice versa.

The bispecific anti-CD123×anti-CD3 antibody (e.g., XmAb14045) and/or one or more additional therapeutic agents, procedures or modalities can be administered during periods of active disorder, or during a period of remission or less active disease. The bispecific anti-CD123×anti-CD3 antibody (e.g., XmAb14045) can be administered before the other treatment, concurrently with the treatment, post-treatment, or during remission of the disorder.

When administered in combination, the bispecific anti-CD123×anti-CD3 antibody (e.g., XmAb14045) and the one or more additional agents (e.g., second or third agent) can be administered in an amount or dose that is higher, lower or the same than the amount or dosage of each agent used individually, e.g., as a monotherapy. In some embodiments, the administered amount or dosage of the bispecific anti-CD123×anti-CD3 antibody (e.g., XmAb14045) and the one or more additional agents (e.g., second or third agent), is lower (e.g., at least about 10%, at least about 20%, at least about 30%, at least about 40%, or at least about 50%) than the amount or dosage of each agent used individually, e.g., as a monotherapy. In other embodiments, the amount or dosage of the bispecific anti-CD123×anti-CD3 antibody (e.g., XmAb14045) and the one or more additional agents (e.g., second or third agent, that results in a desired effect (e.g., treatment of cancer) is lower (e.g., at least about 10%, at least about 20%, at least about 30%, at least about 40%, or at least about 50%) than the amount or dosage of each agent used individually, e.g., as a monotherapy, required to achieve the same therapeutic effect.

In some embodiments, the antibodies are combined with other therapeutic agents, such as anti-allergic agents, anti-nausea agents (or anti-emetics), pain relievers, cytoprotective agents, or any combination thereof.

In some embodiments, a bispecific anti-CD123×anti-CD3 antibody (e.g., XmAb14045) is administered to a human subject in combination with one or more of the following: an anti-TNF or anti-IL6 receptor antibody, a steroid, or an antihistamine (e.g., Benadryl).

In some embodiments, a bispecific anti-CD123×anti-CD3 antibody (e.g., XmAb14045) is administered to a human subject in combination with an antihistamine. In one embodiment, the antihistamine is an H₁ antagonist. In one embodiment, the antihistamine is a first generation H₁ antagonist. In one embodiment, the antihistamine is an ethanolamine. In one embodiment, the ethanolamine is diphenhydramine or carbinoxamine or doxylamine or orphenadrine or bromazine or clemastine or dimenhydrinate. In one embodiment, the antihistamine is diphenhydramine. In one embodiment, the antihistamine is an H₁ antagonist. In one embodiment, the H₁ antagonist is acrivastine, azelastine, bilastine, bromodiphenhydramine, brompheniramine, buclizine, carbinoxamine, cetirizine (Zyrtec®), chlorodiphenhydramine, chlorphenamine, clemastine, cyclizine, cyproheptadine, dexbrompheniramine, dexchlorpheniramine, dimenhydrinate, dimetindene, diphenhydramine, doxylamine, ebastine, embramine, fexofenadine (Allegra®), hydroxyzine (Vistaril®), loratadine (Claritin®), meclizine, mirtazapine, olopatadine, orphenadrine, phenindamine, pheniramine, phenyltoloxamine, promethazine, quetiapine (Seroquel®), rupatadine (Alergoliber®), tripelennamine, and triprolidine, or any combination thereof. In one embodiment, the antihistamine is acrivastine. In one embodiment, the antihistamine is cetirizine. In one embodiment, the antihistamine is diphenhydramine. In one embodiment, the antihistamine is Benadryl®. In one embodiment, the antihistamine is an H₁ inverse agonist. In one embodiment, the H₁ inverse agonist is acrivastine, cetirizine, levocetirizine, desloratadine, pyrilamine, or any combination thereof. In one embodiment, the antihistamine is an H₂ antihistamine. In one embodiment, the H₂ antihistamine is an H₂ antagonist. In one embodiment, the H₂ antihistamine is an H₂ inverse agonist. In one embodiment, the H₂ antihistamine is cimetidine, famotidine, lafutidine, nizatidine, ranitidine, roxatidine, and tiotidine, or any combination thereof.

In some embodiments, a bispecific anti-CD123×anti-CD3 antibody (e.g., XmAb14045) is administered to a human subject who has ALL, in combination with one or more of: Methotrexate (e.g., Abitrexate, Methotrexate LPF, Mexate, Mexate-AQ, Folex, Folex PFS), Nelarabine (e.g., Arranon), Doxorubicin Hydrochloride, Daunorubicin Hydrochloride (e.g., Cerubidine, Rubidomycin) (in combination with cytarabine and anthracycline (daunorubicin or idararubicin), mitoxantrone, fludarabine, cladribine, Clofarabine (e.g., Clofarex or Clolar), Cyclophosphamide (e.g., Cytoxan, Neosar, Clafen), Cytarabine (e.g., Cytosar-U, Tarabine PFS), Dasatinib (e.g., Sprycel), other Brc tyrosine kinase inhibitor, Erwinaze (e.g., Asparaginase Erwinia Chrysanthemi), Imatinib Mesylate (e.g., Gleevec), Ponatinib Hydrochloride (e.g., Iclusig), inotuzumab ozogamicin, https://www.cancer.gov/about-cancer/treatment/drugs/vincristine-sulfate-liposome, Mercaptopurine (e.g., Purinethol, Purixan), Pegaspargase (e.g., Oncaspar), Ponatinib Hydrochloride, Prednisone, Vincristine Sulfate, Vincristine Sulfate Liposome (e.g., Marqibo), Vincasar PFS, Hyper-CVAD, or any combination thereof. The bispecific antibody can be given to human subjects who are undergoing tisagenlecleucel therapy, or additional donor lymphocyte infusions in human subjects that have previously had an allogeneic stem cell transplantation.

In some embodiments, a bispecific anti-CD123×anti-CD3 antibody (e.g., XmAb14045) is administered to a human subject who has AML, in combination with one or more of: Daunorubicin Hydrochloride (e.g., Cerubidine or Rubidomycin) (optionally in combination with cytarabine and anthracycline-daunorubicin or idararubicin), Vyxeos, Idarubicin Hydrochloride (e.g., Idamycin), BCL2 inhibitor (e.g., Venclextra), Cyclophosphamide (e.g., Cytoxan, Clafen, Neosar), Cytarabine (e.g., Cytosar-U, Tarabine PFS), Doxorubicin Hydrochloride, Decitabine (hypomethylating agent), Fludarabine (fludara), Flt3 inhibitors (e.g., sunitinib, sorafenib, midostaurin, lestaurtinib, quizartinib, crenolanib, PLX3397), GCSF (Granulocyte-colony stimulating factor), IDH inhibitors (e.g., IDH1 inhibitors, e.g., AG120 or IDH305); IDH2 inhibitors, e.g., AG221; pan IGH1/IGH2 inhibitors, e.g., AG881), Mitoxantrone Hydrochloride, Thioguanine (e.g., Tabloid), azacitidine (e.g., Vidaza, hypomethylating agent), Vincristine Sulfate (e.g., Vincasar PFS), gemtuzumab ozogamicin, etoposide, enasidenib, or any combination thereof.

In some embodiments, a bispecific anti-CD123×anti-CD3 antibody (e.g., XmAb14045) is administered to a human subject who has CML, in combination with one or more of: Bosutinib (e.g., Bosulif), Busulfan (e.g., Busulfex), Cyclophosphamide (e.g., Clafen, Cytoxan, Neosar), Cytarabine (e.g., Cytosar-U, Tarabine PFS), Dasatinib (e.g., Sprycel), Imatinib Mesylate (e.g., Gleevec), Hydroxyurea (e.g., Hydrea), Ponatinib Hydrochloride (e.g., Iclusig), Mechlorethamine Hydrochloride (e.g., Mustargen), Busulfan (e.g., Myleran), Nilotinib, Omacetaxine Mepesuccinate (e.g., Synribo), and interferon-alpha, or any combination thereof.

### Combination Therapy: Side-Effect Ameliorating Agent

In some embodiments, a bispecific antibody is administered to a human subject in combination with one or more side-effect ameliorating agent(s). Side effects associated with the administration of the CD123×CD3 bispecific antibody, include, but are not limited to cytokine release syndrome (“CRS”). Other possible side effects include hemophagocytic lymphohistiocytosis (HLH), also termed Macrophage Activation Syndrome (MAS). Symptoms of CRS can include high fevers, nausea, transient hypotension, hypoxia, and the like. CRS can include clinical constitutional signs and symptoms such as fever, fatigue, anorexia, myalgias, arthalgias, nausea, vomiting, and headache. CRS can include clinical skin signs and symptoms such as rash. CRS can include clinical gastrointestinal signs and symptoms such as nausea, vomiting and diarrhea. CRS can include clinical respiratory signs and symptoms such as tachypnea and hypoxemia. CRS can include clinical cardiovascular signs and symptoms such as tachycardia, widened pulse pressure, hypotension, increased cardiac output (early) and potentially diminished cardiac output. CRS can include clinical coagulation signs and symptoms such as elevated d-dimer, hypofibrinogenemia with or without bleeding. CRS can include clinical renal signs and symptoms such as azotemia. CRS can include clinical hepatic signs and symptoms such as transaminitis and hyperbilirubinemia. CRS can include clinical neurologic signs and symptoms such as headache, mental status changes, confusion, delirium, word finding difficulty or frank aphasia, hallucinations, tremor, dymetria, altered gait, and seizures.

In one embodiment, the one or more side-effect ameliorating agent(s) include steroids, antihistamines, anti-allergic agents, antinausea agents (or anti-emetics), analgesic agents, antipyretic agents, cytoprotective agents, vasopressor agents, anticonvulsant agents, antiinflammatories, or any combination thereof.

### Combination Therapy: Side-Effect Ameliorating Agent, Steroid

In one embodiment, the side-effect ameliorating agent is a steroid. In one embodiment, the steroid is a corticosteroid. In one embodiment, the corticosteroid is a glucocorticoid. In one embodiment, the corticosteroid is betamethasone, dexamethasone, prednisone, prednisolone, methylprednisolone, triamcinolone, or any combination thereof. In one embodiment, the corticosteroid is hydrocortisone, cortisone, ethamethasoneb, or any combination thereof. In one embodiment, the steroid is fludrocortisone. In one embodiment, the steroid is dexamethasone.

### Combination Therapy: Side-Effect Ameliorating Agent, Antihistamine

In one embodiment, the side-effect ameliorating agent is an antihistamine. In one embodiment, the antihistamine is an H₁ antagonist. In one embodiment, the H₁ antagonist is acrivastine, azelastine, bilastine, bromodiphenhydramine, brompheniramine, buclizine, carbinoxamine, cetirizine (Zyrtec®), chlorodiphenhydramine, chlorphenamine, clemastine, cyclizine, cyproheptadine, dexbrompheniramine, dexchlorpheniramine, dimenhydrinate, dimetindene, diphenhydramine, doxylamine, ebastine, embramine, fexofenadine (Allegra®), hydroxyzine (Vistaril®), loratadine (Claritin®), meclizine, mirtazapine, olopatadine, orphenadrine, phenindamine, pheniramine, phenyltoloxamine, promethazine, quetiapine (Seroquel®), rupatadine (Alergoliber®), tripelennamine, triprolidine, or any combination thereof.

In one embodiment, the antihistamine is acrivastine. In one embodiment, the antihistamine is cetirizine. In one embodiment, the antihistamine is diphenhydramine. In one embodiment, the antihistamine is Benadryl®.

In one embodiment, the antihistamine is an H₁ inverse agonist. In one embodiment, the H₁ inverse agonist is acrivastine, cetirizine, levocetirizine, desloratadine, pyrilamine, or any combination thereof.

In one embodiment, the antihistamine is an H2 antihistamine. In one embodiment, the H2 antihistamine is an H2 antagonist. In one embodiment, the H2 antihistamine is an H2 inverse agonist. In one embodiment, the H2 antihistamine is cimetidine, famotidine, lafutidine, nizatidine, ranitidine, roxatidine, tiotidine, or any combination thereof.

### Combination Therapy: Side-Effect Ameliorating Agent, Anti-Allergy Agent

In one embodiment, the side-effect ameliorating agent is an antiallergy agent. In one embodiment, the side-effect ameliorating agent is antihistamines, glucocorticoids, epinephrine (adrenaline), mast cell stabilizers, antileukotriene agents, anti-cholinergics, decongestants, or any combination thereof. In one embodiment, the side-effect ameliorating agent is a decongestant. In one embodiment, the side-effect ameliorating agent is an adrenaline releasing agent. In one embodiment, the side-effect ameliorating agent is levomethamphetamine, phenylpropanolamine, propylhexedrine (Benzedrex®), loratadine, or any combination thereof. In one embodiment, the side-effect ameliorating agent is an α-adrenergic receptor agonist. In one embodiment, the side-effect ameliorating agent is naphazoline, oxymetazoline, phenylephrine, synephrine, tetryzoline, tramazoline, xylometazoline, or any combination thereof.

### Combination Therapy: Side-Effect Ameliorating Agent, Antinausea Agents (or Anti-Emetic)

In one embodiment, the side-effect ameliorating agent is an antinausea agent. In one embodiment, the side-effect ameliorating agent is an antiemetic agent. In one embodiment, the side-effect ameliorating agent is a 5-HT₃ receptor antagonist. In one embodiment, the side-effect ameliorating agent is a dolasetron (Anzemet®), granisetron (Kytril®, Sancuso®), ondansetron (Zofran®), tropisetron (Setrovel®, Navoban®), palonosetron (Aloxi®), mirtazapine (Remeron®), or any combination thereof. In one embodiment, the side-effect ameliorating agent is a dopamine antagonist. In one embodiment, the side-effect ameliorating agent is a 5-HT₃ receptor antagonist. In one embodiment, the side-effect ameliorating agent is domperidone (Motilium®), olanzapine (Zyprexa®), droperidol, haloperidol, chlorpromazine, prochlorperazine, alizapride, prochlorperazine (Compazine®, Stemzine®, Buccastem®, Stemetil®, Phenotil®), metoclopramide (Reglan®), or any combination thereof. In one embodiment, the side-effect ameliorating agent is a NK1 receptor antagonist. In one embodiment, the side-effect ameliorating agent is aprepitant or fosaprepitant (Emend®), casopitant, rolapitant (Varubi®), or any combination thereof. In one embodiment, the side-effect ameliorating agent is an anticholinergic. In one embodiment, the side-effect ameliorating agent is scopolamine.

Combination Therapy: Side-Effect Ameliorating Agent, Analgesic and/or Antipyretic Agent

In one embodiment, the side-effect ameliorating agent is an analgesic agent. In one embodiment, the side-effect ameliorating agent is an antipyretic agent. In one embodiment, the side-effect ameliorating agent is a salicylate, any derivative thereof, or any combination thereof. In one embodiment, the salicylate is selected from the group consisting of aspirin, diflunisal, salsalate, salicylic acid, any derivative thereof, or any combination thereof. In one embodiment, the salicylate is choline salicylate, magnesium salicylate, sodium salicylate, or any combination thereof. In one embodiment, the side-effect ameliorating agent is aspirin. In one embodiment, the side-effect ameliorating agent is acetaminophen, any derivative thereof. In one embodiment, the side-effect ameliorating agent is an NSAID, any derivative thereof. In one embodiment, the NSAID is a propionic acid derivative. In one embodiment, the NSAID is ibuprofen, dexibuprofen, naproxen, fenoprofen, ketoprofen, dexketoprofen, flurbiprofen, oxaprozin, loxoprofen, any derivative thereof, or any combination thereof. In one embodiment, the NSAID is ibuprofen. In one embodiment, the NSAID is naproxen. In one embodiment, the NSAID is an acetic acid derivative. In one embodiment, the NSAID is indomethacin, tolmetin, sulindac, etodolac, ketorolac, diclofenac, aceclofenac, nabumetone, any derivative thereof, or any combination thereof. In one embodiment, the NSAID is an enolic acid derivative. In one embodiment, the NSAID is piroxicam, meloxicam, tenoxicam, droxicam, lomoxicam, phenylbutazone, any derivative thereof, or any combination thereof. In one embodiment, the NSAID is an anthranilic acid derivative. In one embodiment, the NSAID is mefenamic acid, meclofenamic acid, flufenamic acid, tolfenamic acid, any derivative thereof, or any combination thereof. In one embodiment, the side-effect ameliorating agent is phenazone, metamizole, nabumetone, any derivative thereof, or any combination thereof. In one embodiment, the side-effect ameliorating agent is an opiate. In one embodiment, the side-effect ameliorating agent is codeine, morphine, thebaine, fentanyl, or any combination thereof. In one embodiment, the side-effect ameliorating agent is dihydrocodeine, oxymorphol, oxycodone, oxymorphone, metopon, or any combination thereof.

### Combination Therapy: Side-Effect Ameliorating Agent, Cytoprotective Agent

In one embodiment, the side-effect ameliorating agent is a cytoprotective agent. In one embodiment, the side-effect ameliorating agent is an aminothiol compound. In one embodiment, the side-effect ameliorating agent is amifostine. In one embodiment, the side-effect ameliorating agent is bleomycin, dexrazoxane, coenzyme M, or any combination thereof.

### Combination Therapy: Side-Effect Ameliorating Agent, Vasopressor Agent

In one embodiment, the side-effect ameliorating agent is a vasopressor agent. In one embodiment, the vasopressor agent is norepinephrine, phenylephrine, epinephrine, ephedrine, dopamine, vasopressin, or any combination thereof. In one embodiment, the vasopressor agent is dobutamine, midodrine, amezinium, or any combination thereof.

### Combination Therapy: Side-Effect Ameliorating Agent, Anticonvulsant Agent

In one embodiment, the side-effect ameliorating agent is an anticonvulsant agent. In one embodiment, the anticonvulsant is an aldehyde. In one embodiment, the aldehyde is paraldehyde. In one embodiment, the anticonvulsant is an aromatic allylic alcohol. In one embodiment, the aromatic allylic alcohol is stiripentol. In one embodiment, the anticonvulsant is a barbiturate. In one embodiment, the barbiturate is phenobarbital, primidone, methylphenobarbital, barbexaclone, or any combination thereof. In one embodiment, the anticonvulsant is a benzodiazepine. In one embodiment, the benzodiazepine is clobazam, clonazepam, clorazepate, diazepam, midazolam, lorazepam, nitrazepam, temazepam, nimetazepam, or any combination thereof. In one embodiment, the anticonvulsant is a carboxamide. In one embodiment, the carboxamide is carbamazepine, oxcarbazepine, eslicarbazepine acetate or any combination thereof. In one embodiment, the anticonvulsant is a fatty acid. In one embodiment, the fatty acid is a valproate. In one embodiment, the valproate is valproic acid, sodium valproate, divalproex sodium, or any combination thereof. In one embodiment, the valproate is vigabatrin, progabide, and tiagabine. In one embodiment, the anticonvulsant is a fructose derivative. In one embodiment, the fructose derivative is topiramate. In one embodiment, the anticonvulsant is a GABA analog. In one embodiment, the GABA analog is gabapentin, pregabalin, or any combination thereof. In one embodiment, the anticonvulsant is a hydantoin. In one embodiment, the hydantoin is ethotoin, phenytoin, mephenytoin, fosphenytoin, or any combination thereof. In one embodiment, the anticonvulsant is an oxazolidinedione. In one embodiment, the oxazolidinedione is paramethadione, trimethadione, ethadione, or any combination thereof. In one embodiment, the anticonvulsant is a propionate. In one embodiment, the anticonvulsant is a pyrimidinedione. In one embodiment, the anticonvulsant is a pyrrolidine. In one embodiment, the pyrrolidine is brivaracetam, etiracetam, levetiracetam, seletracetam, or any combination thereof. In one embodiment, the anticonvulsant is levetiracetam. In one embodiment, the anticonvulsant is a succinimide. In one embodiment, the succinimide is ethosuximide, phensuximide, mesuximide, or any combination thereof. In one embodiment, the anticonvulsant is a sulfonamide. In one embodiment, the succinimide is acetazolamide, sultiame, methazolamide, zonisamide, or any combination thereof. In one embodiment, the anticonvulsant is a triazine. In one embodiment, the triazine is lamotrigine. In one embodiment, the anticonvulsant is a urea. In one embodiment, the urea is pheneturide, phenacemide, or any combination thereof. In one embodiment, the anticonvulsant is a valproylamide. In one embodiment, the anticonvulsant is a valproylamide. In one embodiment, the valproylamide is valpromide, valnoctamide, or any combination thereof. In one embodiment, the anticonvulsant is perampanel, stiripentol, pyridoxine, or any combination thereof.

### Combination Therapy: Side-Effect Ameliorating Agent, TNFα Inhibitor

In one embodiment, the side-effect ameliorating agent is an anti-inflammatory agent. In one embodiment, the side-effect ameliorating agent is a TNF-α inhibitor. In one embodiment, the TNF-α inhibitor is an antibody. Examples of an anti-TNFα antibody molecule such as, infliximab (Remicade®), adalimumab (Humira®), certolizumab pegol (Cimzia®), golimumab (Simponi®), or any combination thereof. Another example of a TNFα inhibitor is a fusion protein such as entanercept (Enbrel®). In one embodiment, the TNF-α inhibitor is a small molecule. Small molecule inhibitor of TNFα include, but are not limited to, xanthine derivatives (e.g. pentoxifylline), bupropion, or any combination thereof.

### Combination Therapy: Side-Effect Ameliorating Agent, IL6 Inhibitor

In one embodiment, the side-effect ameliorating agent is an anti-inflammatory agent. In one embodiment, the side-effect ameliorating agent is a IL-6 inhibitor. An example of an IL-6 inhibitor is an anti-IL-6 antibody molecule such as tocilizumab (toc), sarilumab, elsilimomab, CNTO 328, ALD518/BMS-945429, CNTO 136, CPSI-2364, CDP6038, VX30, ARGX-109, FE301, FM101, or any combination thereof. In one embodiment, the anti-IL-6 antibody molecule is tocilizumab.

The methods described herein can comprise administering a bispecific antibody described herein to a human subject and further administering one or more agents to manage elevated levels of a soluble factor resulting from treatment with a bispecific antibody. In one embodiment, the soluble factor elevated in the human subject is one or more of IFN-γ, TNFα, IL-2 and IL-6. In an embodiment, the factor elevated in the human subject is one or more of IL-1, GM-CSF, IL-10, IL-8, IL-5 and fraktalkine. Therefore, an agent administered to treat this side effect can be an agent that neutralizes one or more of these soluble factors. In one embodiment, the agent that neutralizes one or more of these soluble forms is an antibody or antigen binding fragment thereof. Examples of such agents include, but are not limited to a steroid (e.g., corticosteroid), an inhibitor of TNFα, and inhibitor of IL-1R, and an inhibitor of IL-6. An example of an IL-1R based inhibitor is anakinra.

In one embodiment, the side-effect ameliorating agent is one that reduces an immune-mediated side effect. Exemplary immune-mediated side effects include, but are not limited to pneumonitis, colitis, hepatitis, nephritis and renal dysfunction, hypothyroidism, hyperthyroidism, and endocrinopathies (e.g., hypophysitis, Type 1 diabetes mellitus and thyroid disorders such as hypothyroidism and hyperthyroidism). In one embodiment, the side-effect ameliorating agent reduces embryofetal toxicity.

In an embodiment, the human subject can be administered an antipyretic agent. In an embodiment, the human subject can be administered an analgesic agent.

### Side-Effect Combinations and Amounts

In one embodiment, a steroid is administered prior to the bispecific antibody. In one embodiment, the steroid is administered in an amount of between about 5 mg and 30 mg. In one embodiment, the steroid described herein is administered in an amount of between about 5 mg and 25 mg. In one embodiment, the steroid is administered in an amount of between about 5 mg and 15 mg. In one embodiment, the steroid is administered in an amount of between about 8 mg and 12 mg. In one embodiment, the steroid is administered in an amount of about 10 mg. In one embodiment, the steroid is administered in an amount of 10 mg. In one embodiment, the steroid is administered in an amount of between about 18 mg and 22 mg. In one embodiment, the steroid is administered in an amount of about 20 mg. In one embodiment, the steroid is administered in an amount of 20 mg. In one embodiment, the steroid is dexamethasone. In one embodiment, the steroid is dexamethasone and is administered in an amount of about 10 mg. In one embodiment, the steroid is dexamethasone. In one embodiment, the steroid is dexamethasone and is administered in an amount of about 20 mg.

In one embodiment, an antihistamine is administered prior to the bispecific antibody. In one embodiment, the antihistamine is an H₁ antagonist. In one embodiment, the H₁ antagonist is a first generation H₁ antagonist. In one embodiment, the antihistamine is an ethanolamine. In one embodiment, the ethanolamine is diphenhydramine, carbinoxamine, doxylamine, orphenadrine, bromazine, clemastine, dimenhydrinate, or any combination thereof. In one embodiment, the antihistamine is diphenhydramine. In one embodiment, the antihistamine is diphenhydramine. In one embodiment, the antihistamine is administered in an amount of between about 20 mg and 60 mg. In one embodiment, the antihistamine is administered in an amount of between about 20 mg and 30 mg. In one embodiment, the antihistamine is administered in an amount of about 25 mg. In one embodiment, the antihistamine is administered in an amount of 25 mg. In one embodiment, the antihistamine is administered in an amount of between about 40 mg and 60 mg. In one embodiment, the antihistamine is administered in an amount of between about 45 mg and 55 mg. In one embodiment, the antihistamine is administered in an amount of about 50 mg. In one embodiment, the antihistamine is administered in an amount of 50 mg. In one embodiment, the antihistamine is diphenhydramine and the amount of between about 20 mg and about 30 mg. In one embodiment, the antihistamine is diphenhydramine and the amount is about 25 mg.

In one embodiment, acetaminophen is administered prior to the bispecific antibody. In one embodiment, acetaminophen is administered in an amount of between about 100 mg and 1000 mg. In one embodiment, acetaminophen is administered in an amount of between about 400 mg and 600 mg. In one embodiment, acetaminophen is administered in an amount of about 500 mg. In one embodiment, acetaminophen is administered in an amount of 500 mg. In one embodiment, acetaminophen is administered in an amount of between about 500 mg and 800 mg. In one embodiment, acetaminophen is administered in an amount of between about 550 mg and 750 mg. In one embodiment, acetaminophen is administered in an amount of between about 600 mg and 700 mg. In one embodiment, acetaminophen is administered in an amount of about 650 mg. In one embodiment, acetaminophen is administered in an amount of 650 mg. In one embodiment, the acetaminophen described herein is administered in an amount of 650 mg.

In one embodiment, a steroid, an H₁ antagonist, and acetaminophen are administered prior to the bispecific antibody. In one embodiment, dexamethasone, an H₁ antagonist, and acetaminophen are administered prior to the bispecific antibody. In one embodiment, a steroid, diphenhydramine, and acetaminophen are administered prior to the bispecific antibody. In one embodiment, dexamethasone, diphenhydramine, and acetaminophen are administered prior to the bispecific antibody. In one embodiment, dexamethasone is administered in an amount of about 10 mg or about 20 mg, diphenhydramine is administered in an amount of about 25 mg, and acetaminophen is administered in an amount of about 650 mg prior to the bispecific antibody.

In one embodiment, an antinausea agent is administered prior to the bispecific antibody. In one embodiment, the antinausea agent is a 5-HT₃ receptor antagonist. In one embodiment, the 5-HT₃ receptor antagonist is administered in an amount of between about 5 mg and 30 mg. In one embodiment, the 5-HT₃ receptor antagonist is administered in an amount of between about 5 mg and 15 mg. In one embodiment, the 5-HT₃ receptor antagonist is administered in an amount of between about 5 mg and 10 mg. In one embodiment, the 5-HT₃ receptor antagonist is administered in an amount of about 8 mg. In one embodiment, the 5-HT₃ receptor antagonist is administered in an amount of 8 mg. In one embodiment, the 5-HT₃ receptor antagonist is ondansetron.

In one embodiment, an NK1 receptor antagonist is administered prior to the bispecific antibody. In one embodiment, the NK1 receptor antagonist is administered in an amount of between about 100 mg and 300 mg. In one embodiment, the NK1 receptor antagonist is administered in an amount of between about 125 mg and 200 mg. In one embodiment, the NK1 receptor antagonist is administered in an amount of between about 125 mg and 175 mg. In one embodiment, the NK1 receptor antagonist is administered in an amount of about 150 mg. In one embodiment, the NK1 receptor antagonist is administered in an amount of 150 mg. In one embodiment, the NK1 receptor antagonist is aprepitant, fosaprepitant, or combination thereof. In one embodiment, the NK1 receptor antagonist is fosaprepitant dimeglumine.

Whereas particular embodiments of the invention have been described above for purposes of illustration, it will be appreciated by those skilled in the art that numerous variations of the details can be made without departing from the invention as described in the appended claims.

Whereas particular embodiments of the invention have been described above for purposes of illustration, it will be appreciated by those skilled in the art that numerous variations of the details can be made without departing from the invention as described in the appended claims.

### EXAMPLES

Examples are provided below to illustrate the methods described throughout. These examples are not meant to constrain the methods to any particular application or theory of operation. For all constant region positions discussed, numbering is according to the EU index as in Kabat (Kabat et al., 1991, Sequences of Proteins of Immunological Interest, 5th Ed., United States Public Health Service, National Institutes of Health, Bethesda, entirely incorporated by reference). A skilled artisan will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are outlined in U.S. Pat. Appl. Pubs. 2015/0307629, 2014/0288275, 2014/294823, 2016/0229924, and 2016/0355608.

### Example 1

### XmAb14045 Treatment Plan

This is a multicenter, open-label, multi-dose, single-arm, Phase 1, dose-escalation study of XmAb14045. The dose of XmAb14045 will be administered IV over a 2-hr infusion period. Modifications of the dose infusion period can occur based on any observed infusion toxicity.

XmAb14045 is a humanized bsAb that binds both CD123 and CD3. The XmAb14045 pharmaceutical composition is a sterile liquid supplied in single-use glass vials. Each vial is filled with 1.1 mL of pharmaceutical composition that contains 1.0 mg/mL (±5%) of XmAb14045, in 10 mM sodium citrate, 150 mM sodium chloride, and 0.04% (w/v) polysorbate-80 at pH 5.5. Each product vial is intended to deliver 1.0 mL of drug solution.

IV Solution Stabilizer will be supplied in single-use glass vials. Each vial is filled with 10.5 mL of a solution containing 250 mM sodium citrate, and 1.0% (w/v) polysorbate-80 at pH 5.5. Each product vial is intended to deliver 10.0 mL of drug solution.

Prior to administration, XmAb14045 will be diluted to the required final concentration in one or more ethylene/polypropylene copolymer infusion bags (Excel™, B. Braun) containing 250 mL 0.9% Sodium Chloride Injection, USP after replacement of 10 mL with 10.0 mL IV Solution Stabilizer. After dilution, the bag containing XmAb14045 should be gently inverted 2 to 3 times to mix the solution. The bag should not be shaken.

Prior to each dose of XmAb14045, human subjects should receive:
Dexamethasone 10-20 mg IV, approximately 1 hour prior to each weekly dose of XmAb14045 in Parts A and B. In Part C, dexamethasone should be administered before the C1D1 and C1D15 dose and may be omitted on subsequent doses, unless significant CRS symptoms occur.
Acetaminophen 650 mg orally, approximately 30 min before infusion
Diphenhydramine 25 mg PO or IV, approximately 30-60 min before infusion

This study will be conducted in 3 parts: Part A, dosing cohorts that establish a MTD/RD for the first infusion; followed by Part B, dosing cohorts that establish a MTD/RD for the second (and subsequent infusions) after human subjects receive their first infusion at the dose determined in Part A; and Part C (enrolled concurrently with Parts A and B), dosing cohorts that establish a MTD/RD for a dosing schedule of 3 times per week dosing for the 1st 2 weeks of therapy (Induction), followed by once a week dosing (Consolidation).

Part A: Human subjects will be enrolled in up to 15 consecutive dose cohorts (0.003, 0.01, 0.03, 0.075, 0.15, 0.3, 0.5, 0.75, 1.3, 2.3, 4.0, 7.0, 12.0, 20.0, and 35.0 μg/kg) with initial accelerated titration for the first 3 cohorts. The first 3 cohorts will consist of 1 human subject each until there is evidence of a ≥Grade 2 toxicity, and the remaining cohorts will enroll at least 3 human subjects each in a classic 3+3 dose escalation scheme. Human subjects will be admitted for 3 days for the first and fourth doses (and 2 days for the second dose, if admission is necessary to collect cytokine/inflammatory factors for the 8 hr post-infusion timepoint) for observation, PK, PD, and laboratory assessment. Within each ascending dose cohort (Cohorts 1A-8A), human subjects will be given XmAb14045 IV over 2 hr, once every 7 days, for a total of 4 doses in each 28-day cycle. The initial treatment period will include 2 cycles. Disease assessments occurred at the end of odd-numbered cycles. After the MTD and/or RD dose is reached, the cohort can be expanded by up to an additional 12 human subjects to obtain additional safety data.

Part B: An attempt will be made to escalate to higher doses for the second and subsequent drug infusions. Human subjects will be admitted for 3 days for the first and fourth dose as in Part A, but also for the escalated second dose (Day 8) for observation, PK, PD, and cytokine assessment.

Part C: Human subjects will be enrolled in up to 8 consecutive dose cohorts, with the initial dose level based on the highest tolerable dose level achieved in Part A or B at that point in time. Administration of XmAb14045 will be divided into Induction (C1D1-C1D14) and Consolidation phases (C1D15 and after). Induction will consist of 6 2-hour infusions (Days 1, 3, 5, 8, 10, and 12) starting at a dose one-third of the highest once a week dose level from Part A, that has been assessed as tolerable/safe by the DERC (Dose Escalation Review Committee, a group of study investigators as well as the study medical monitor) and Consolidation will consist of once a week 2-hour infusions (C1D15 and C1D22, as well as all subsequent infusions) at the full highest once a week dose level from Part A, that has been assessed as tolerable/safe by the DERC.

Part C cohorts will enroll at least 3 human subjects each in a classic 3+3 dose escalation scheme. Human subjects will be admitted for 3 days during the first through second doses, as well as for the eighth dose for observation, PK, PD, and laboratory assessment.

If all 3 human subjects tolerate the initial Part C dosing cohort without experiencing DLT (and the DERC agrees), enrollment will begin on the next higher cohort, as defined in Table 4. The initial treatment period will include 2 cycles. After the MTD and/or RD dose is reached, the cohort can be expanded by up to an additional 12 human subjects to obtain additional safety data. If a MTD/RD is identified in the Consolidation phase, escalation in the Induction phase can continue until an MTD/RD is also identified.

The dose to be administered to the human subject for all cohorts will be calculated based on baseline (Day −1) weight measurement in kg. Following the first dose, subsequent doses will only be modified if the human subject's weight changes by more than 10% from the Day −1 weight at which point it will be recalculated for that infusion day using the current weight. For human subjects whose weight exceeds 100 kg, the dose of XmAb14045 will be calculated based on a weight of 100 kg and will not be calculated based upon the human subject's actual body weight.

A dose escalation schema will be employed in single dose level cohorts for Part A and sequentially increasing second and subsequent infusion dosing cohorts for Part B. Dose escalation will continue in both Parts A and B until the MTD and/or RD for further study has been identified or until a dose of 35.0 μg/kg has been reached, whichever comes first. Intrapatient dose escalation was allowed.

Human subjects will receive two 28-day cycles of therapy (8 once a week doses in Part A and B; and 3 doses per week×2 weeks followed by 6 once a week doses for Part C). In the absence of unacceptable study drug-related toxicity, human subjects can receive additional cycles of therapy if there is clinical benefit (as assessed by the investigator). Doses will be administered on Days 1, 8, 15, and 22 of each cycle, except as noted for Part C. Dosing can be delayed in the presence of drug-related toxicities. Human subjects who complete 4 doses for Parts A and B (8 doses for Part C) of XmAb14045 and undergo the planned safety evaluations through Day 22 (+up to 2 days to allow for minor scheduling changes and dosing delays) will be considered to have sufficient safety data/follow-up for identification of DLTs. If the MTD and/or RD are not reached, dose escalation to the next dose cohort will occur following review by the DERC. Human subjects will be followed for at least 4 weeks after treatment is discontinued or until disease progression requiring therapy, stem cell transplantation or the occurrence of death, whichever comes first. Following the last study visit, information regarding disease status and survival will be collected by the investigational sites by either clinic visit or telephone contact for an additional 6 months, or until the occurrence of death.

### Dose Escalation Scheme Part A

In Part A, dose level increases will initially proceed according to an accelerated titration design (see Table 1). This design allows for more efficient dose escalation while maintaining safety standards by implementing conservative triggers for cohort expansion during the accelerated escalation phase, and can limit the number of human subjects exposed to potentially sub-therapeutic doses of XmAb14045.

TABLE 1
Study Cohorts - Part A

| | | | Human |
|---|---|---|---|
| | Cohort | Planned Dose | subjects |
| Part A | 1A | 3 ng/kg (0.003 μg/kg) | 1 (+2 + 3) |
| | 2A | 10 ng/kg (0.01 μg/kg) | 1 (+2 + 3) |
| | 3A | 30 ng/kg (0.03 μg/kg) | 1 (+2 + 3) |
| | 4A | 75 ng/kg (0.075 μg/kg) | 3 (+3) |
| | 5A | 150 ng/kg (0.150 μg/kg) | 3 (+3) |
| | 6A | 300 ng/kg (0.3 μg/kg) | 3 (+3) |
| | 7A | 500 ng/kg (0.5 μg/kg) | 3 (+3) |
| | 8A | 750 ng/kg (0.75 μg/kg) | 3 (+3) |
| | 9A | 1.3 μg/kg | 3 (+3) |
| | 10A | 2.3 μg/kg | 3 (+3) |
| | 11A | 4.0 μg/kg | 3 (+3) |
| | 12A | 7.0 μg/kg | 3 (+3) |
| | 13A | 12.0 μg/kg | 3 (+3) |
| | 14A | 20.0 μg/kg | 3 (+3) |
| | 15A | 35.0 μg/kg | 3 (+3) |
| | Expansion-A | At MTD or recommended | Up to 12 |
| | | first infusion dose | |

MTD = maximum tolerated dose.

During the initial accelerated dose escalation phase (Cohorts 1A, 2A, and 3A), dose escalation can occur after treatment of 1 human subject per cohort provided that there is no ≥Grade 2 toxicity during Cycle 1 and the human subject has met minimum safety assessment requirements (see Table 2). When a human subject experiences a ≥Grade 2 toxicity during the dose escalation safety assessment period, the accelerated escalation phase will end, the standard dose escalation phase will begin, and the cohort in which the event(s) occurred will be expanded to a total of at least 3 human subjects (2 additional human subjects will be enrolled).

TABLE 2
Dose Escalation Scheme
Accelerated Dose Escalation Phase

| | Number of Human | |
|---|---|---|
| | Subjects Enrolled and | |
| Number of Human | Assessable for Safety | |
| Subjects with at Least | Following Four Doses | |
| One Event ≥ Grade 2 | of XmAb14045 | Escalation Decision |
| 0 | 1 | Escalate to the next |
| | | higher dose level |
| 1 | 1 | Enroll 2 additional |
| | | human subjects on the |
| | | same dose level and |
| | | revert to Standard Dose |
| | | Escalation (3 + 3) design |
| | | below. |

Standard Dose Escalation Phrase

| | Number of Human | |
|---|---|---|
| | Subjects Enrolled and | |
| Number of Human | Assessable for Safety | |
| Subjects with at Least | Following Four Doses | |
| One DLT | of XmAb14045 | Escalation Decision |
| 0 | 3 | Escalate to the next |
| | | higher dose level |
| 1 | 3 | Enroll 3 additional |
| | | human subjects on the |
| | | same dose level |
| 1 | 6 | Escalate to the next |
| | | higher dose level |
| 2 | 3 or 6 | No dose escalation can |
| | | occur; MTD has been |
| | | surpassed. The next |
| | | lower dose level should |
| | | be expanded. |

DLT = dose-limiting toxicity;
MTD = maximum tolerated dose

From this cohort forward (or beginning with Cohort 4A [0.075 μg/kg], whichever comes first) the standard 3+3 dose escalation rules will apply:

If zero of 3 human subjects have a DLT, then dose escalation to the next level will occur.

If 1 of 3 human subjects has a DLT, then the cohort will be further expanded to a total of 6 human subjects or until a second human subject in the cohort experiences a DLT. If there are no additional human subjects with a DLT, then dose escalation to the next higher dose level will occur.

The MTD is defined as the highest dose level at which no more than 1 human subject experiences DLT out of 6 human subjects assessable for toxicity at that dose level. Any cohort with 2 or more human subjects experiencing a DLT will have exceeded the MTD and there will be no further dose escalation. The dose level below the cohort at which 2 or more human subjects with DLT occurred will be expanded to at least 6 to delineate the MTD.

Before a dose-escalation decision can be reached, at least 1 human subject (in the accelerated dose escalation phase of the study) or 3 human subjects (in the standard escalation phase of the study) must meet all requirements for dose escalation safety assessment.

For the purpose of determining the incidence of DLT and defining the MTD and/or recommended dosing of XmAb14045 for future study, only human subjects who experience DLT and those with sufficient safety data/follow-up will be evaluated. Human subjects who complete 4 doses of XmAb14045 and undergo the planned safety evaluations through Day 22 (up to +2 days to account for minor scheduling changes and dosing delays) will be considered to have sufficient safety data/follow-up. Human subjects who withdraw from study before completing Day 22 of treatment for reasons unrelated to study drug toxicity will be considered to have inadequate data to support dose escalation. In such cases, replacement human subjects will be enrolled to receive the same dose of XmAb14045 as the human subjects who withdraw prematurely.

The decision to advance dosing to the next cohort level will be made by the DERC after review of all required dose escalation safety assessment data from human subjects in a cohort. PK and ADA data cannot be routinely available during the safety assessment period as these samples can be batched for analysis so that a more uniform drug exposure analysis and ADA analysis can be performed across all study samples. However, if a human subject safety issue arises and the treating physician feels that information around drug exposure and/or ADA analysis would be useful information in determining the treatment plan for the human subjects, PK and ADA analysis can be performed on the human subject samples that have been collected to date.

Once the MTD (or RD for further study) is identified, the MTD/RD dose level can be further expanded up to an additional 12 human subjects (up to a total MTD/RD cohort of 18 human subjects) to further assess safety and PK.

The dose escalation scheme can be modified (e.g., smaller increases or decreases in dose level can be permitted, additional human subjects in a cohort can be enrolled, infusion duration and scheduling can be modified) based on available PK and PD data, and the type and severity of toxicities observed in this trial, upon agreement of the DERC.

### Dose Escalation Scheme—Part B—

In Part B, the Day 1 dose will be fixed at the level determined in Part A. The second dose will be escalated and maintained for subsequent doses. Dosing cohorts will be defined relative to the MTD/RD determined in Part A.

TABLE 3
Study Cohorts - Part B

| | | | | | | Human |
|---|---|---|---|---|---|---|
| | Cohort | Day 1 | Day 8 | Day 15 | Day 22 | Subjects |
| Part B | −1B | X | X + 1 | X + 1 | X + 1 | 3 (+3) |
| | 2B | X | X + 2 | X + 2 | X + 2 | 3 (+3) |
| | 3B | X | X + 3 | X + 3 | X + 3 | 3 (+3) |
| | 4B | X | X + 4 | X + 4 | X + 4 | 3 (+3) |
| | 5B | X | X + 5 | X + 5 | X + 5 | 3 (+3) |
| | 6B | X | X + 6 | X + 6 | X + 6 | 3 (+3) |
| | 7B | X | X + 7 | X + 7 | X + 7 | 3 (+3) |

| | | |
|---|---|---|
| Expansion-B | At MTD or RD cohort | Up to 12 |

MTD = maximum tolerated dose;
RD = recommended dose;
X = Part A MTD/RD

Dose escalation will proceed as described for the standard 3+3 scheme noted in Part A and with the same dosing levels (0.003, 0.01, 0.03, 0.075, 0.15, 0.3, 0.5, 0.75, 1.3, 2.3, 4.0, 7.0, 12.0, 20.0, and 35.0 μg/kg) however the Day 1 infusion dose will always be the MTD/RD determined in Part A (denoted as “X” in Table 3). Dose escalation on each Part B cohort will be based on this starting point. For example, if the MTD/RD from Part A is 0.03 μg/kg, the first infusion in Cohort 1B will be 0.03 μg/kg and the second and subsequent infusions will be at 0.075 μg/kg (i.e. X+1).

A minimum of 3 human subjects will be enrolled in each cohort. As in Part A, no two human subjects will start treatment with XmAb14045 on the same day. If all 3 human subjects tolerate a cohort without experiencing DLT (and the DERC agrees), enrollment will begin on the next higher cohort. If at any time through Day 22 (up to +2 days to account for minor scheduling changes and dosing delays) a DLT occurs, or if the Medical Monitor determines that additional safety data is needed for a given dose cohort, 3 additional human subjects will be added to the cohort. If there is an additional DLT among the 6 human subjects on the cohort, the previous dosing cohort will be expanded to 6 to establish a MTD and/or RD. If this occurs on Cohort 1B, the next 3 human subjects will be enrolled on Cohort-1B. If there are no further DLTs among the 3 additional human subjects, another 3 human subjects will be added to the cohort. If there is an additional DLT, then the MTD/RD and schedule established in Part A will be recommended for further study.

The dose escalation scheme can be modified (e.g., smaller increases or decreases in dose level can be permitted, additional human subjects in a cohort can be enrolled, infusion duration and scheduling can be modified) based on available PK and PD data, and the type and severity of toxicities observed, upon agreement of the DERC.

### Dose Escalation Scheme—Part C

Accrual into Part C cohorts will begin as soon as feasible. Administration of XmAb14045 will be divided into Induction (C1D1-C1D14) and Consolidation phases (C1D15 and after). Induction will be 3 infusions per week (Cycle 1, Days 1, 3, 5, 8, 10 and 12) (Induction dose), given IV over 2 hours. From C1D15 on, administration will be once a week (Consolidation), also administered over 2 hours. The Induction phase of the first Part C cohort will start at a dose of one-third of the highest once a week dose level from Part A (not to exceed a C1D1 dose of 0.75 μg/kg), that has been assessed as tolerable/safe by the DERC (0.43 μg/kg) and Consolidation will consist of once a week 2-hour infusions (C1D15 and C1D22, as well as all subsequent infusions) at the full highest once a week dose level from Part A, that has been assessed as tolerable/safe by the DERC (1.3 μg/kg).

See Table 4 for specific doses and planned cohort dose escalation.

TABLE 4
Study Cohorts-Part C

| | | C1D1 | Induction | | |
|---|---|---|---|---|---|
| | | Dose | Dose | Consolidation | Human |
| | Cohort | (μg/kg) | (μg/kg) | Dose (μg/kg) | Subjects |
| Part C | 8C | 0.25 | 0.25 | 0.75 | 3 (+3) |
| | 9C | 0.43 | 0.43 | 1.3 | 3 (+3) |
| | 10C | 0.75 | 0.77 | 2.3 | 3 (+3) |
| | 11C | 0.75 | 1.3 | 4.0 | 3 (+3) |
| | 12C | 0.75 | 2.3 | 7.0 | 3 (+3) |
| | 13C | 0.75 | 4.0 | 12.0 | 3 (+3) |
| | 14C | 0.75 | 6.7 | 20.0 | 3 (+3) |
| | 15C | 0.75 | 11.7 | 35.0 | 3 (+3) |

| | | |
|---|---|---|
| Expansion-C | At Part C MTD or RD | Up to 12 |

A minimum of 3 human subjects will be enrolled in each cohort. As in Part A and B, no two human subjects will start treatment with XmAb14045 on the same day. If all 3 human subjects tolerate a cohort without experiencing DLT (and the DERC agrees), enrollment will begin on the next higher cohort. If at any time through Day 22 (up to +2 days to account for minor scheduling changes and dosing delays) a DLT occurs, or if the Medical Monitor determines that additional safety data is needed for a given dose cohort, 3 additional human subjects will be added to the cohort. If there is an additional DLT among the 6 human subjects on the cohort, the previous dosing cohort will be expanded to 6 to establish a MTD and/or RD.

The dose escalation scheme can be modified (e.g., smaller increases or decreases in dose level can be permitted, additional human subjects in a cohort can be enrolled, infusion duration and scheduling can be modified) based on available PK and PD data, and the type and severity of toxicities observed, upon agreement of the DERC.

Changes that can only be made with a protocol amendment will include:
Dose escalation in either the Induction or Consolidation phases that are more rapid than shown in Table 4.
Administration of more than 4 infusions per week
Administration of more than 2 weeks of greater than once a week dosing
Exceeding the highest tolerable C1D1 dose achieved in Part A as the C1D1 induction dose in Part C.

### Results:

At data cut-off, 95 human subjects have been treated, 94 with relapsed/refractory AML and 1 with B-ALL. Human subjects had a median age of 62 years (range of 18-85 yrs) and were heavily pretreated (median of 3 prior therapies [range 1-8]). CRS or its component symptoms were the most common treatment-emergent adverse event (TEAE). CRS episodes began within approximately 1-4 hours of the start of drug infusion and occurred in 76 of 95 human subjects (80%). Grade ≥3 CRS was only seen at doses of 1,300 ng/kg or 2,300 ng/kg, and on the first dose, with one exception. No myelosuppression requiring dose modification was observed. Two human subjects had evidence of mild tumor lysis syndrome.

Based on published data from December 2018, from a subset of these human subjects administered according to Cohorts 9A, 10A, 1B and 2B, single agent antileukemic activity was documented with a best response of CR (2) or CRi (3) in 5/18 human subjects (CR/CRi rate 27.8%) treated at the two highest dose levels studied to date (1,300 ng/kg or 2,300 ng/kg once a week); no CR, CRi, or morphologic leukemia-free state (MLFS) responses were seen at lower doses. Antileukemic activity occurred quickly; all responders had achieved at least an MLFS response after 4 doses (1 cycle). Stable disease lasting for greater than 3 months occurred in an additional 3 patients. Reduction of marrow blasts occurred in 56% of patients. Three responders were bridged to stem cell transplantation. Median duration of response is 15.4 weeks (range 9.1-20.3+). Excluding CRS-related events, additional TEAEs occurring in >10% of patients included chills (39%), fever (27%), tachycardia (21%), increased ALT (18%), anemia (17%), hypotension (17%), fatigue (15%), hypertension (14%), increased AST (12%), lymphopenia (11%), nausea (11%), and vomiting (11%). Recurrent infusion-related back or head pain occurred in 4 human subjects and were managed with analgesics. Grade 3 transaminase elevation occurring within 24 hours of XmAb14045 infusion was seen in 5 patients with all resolved within 7 days, and most often occurring with the first dose of XmAb14045. Only one patient developed hyperbilirubinemia (Grade 1). The December 2018 published data subset had 66 patients; the median age was 61 years (range 18-85); 46% were female; 100% had an AML diagnosis; median time since initial diagnosis was 49 weeks (range 3-879); the median number of prior therapies is 3 (range 1-8); 30% of human subjects had a history of hematopoietic stem cell transplantation; 86% were refractory to last therapy; 5% of human subjects had an ELN Risk Category of Favorable; 33% of human subjects had an ELN Risk Category of Intermediate; 53% of human subjects had an ELN Risk Category of Adverse; 9% of human subjects had an ELN Risk Category of Unknown. 11% of human subjects had secondary leukemia. From the data, it can be seen that XmAb14045 at the dose and schedule studied was well tolerated and had clinical activity in relapsed AML. The Antibody construct with full-length Fc region permitted weekly dosing. Cytokine release syndrome was the primary toxicity of XmAb14045; management with premedication and the use of a priming dose and step-up dosing was effective in limiting its severity. No clear evidence of myelosuppression was observed even after prolonged administration. Clinically significant responses were achieved in relapsed/refractory AML allowing allogeneic stem cell transplant.

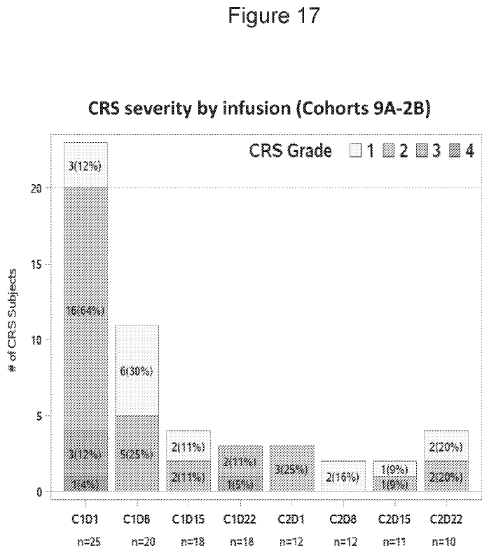

Based on published data from December 2018, from a subset of these human subjects, CRS severity by infusion (Cohorts 9A-2B) is described in FIG. 17. No premedication was given for Cohorts 1A-3A. Standard premedications were added for Cohort 4A (75 ng/kg): Dexamethasone 10-20 mg IV; Diphenhydramine 50 mg po; Acetaminophen 500 mg po. All episodes of CRS began within 1-4 hours of the start of drug infusion and usually resolved within 1-4 hours. CRS was generally more severe on the initial dose, accounting for most ≥Grade 3 episodes.

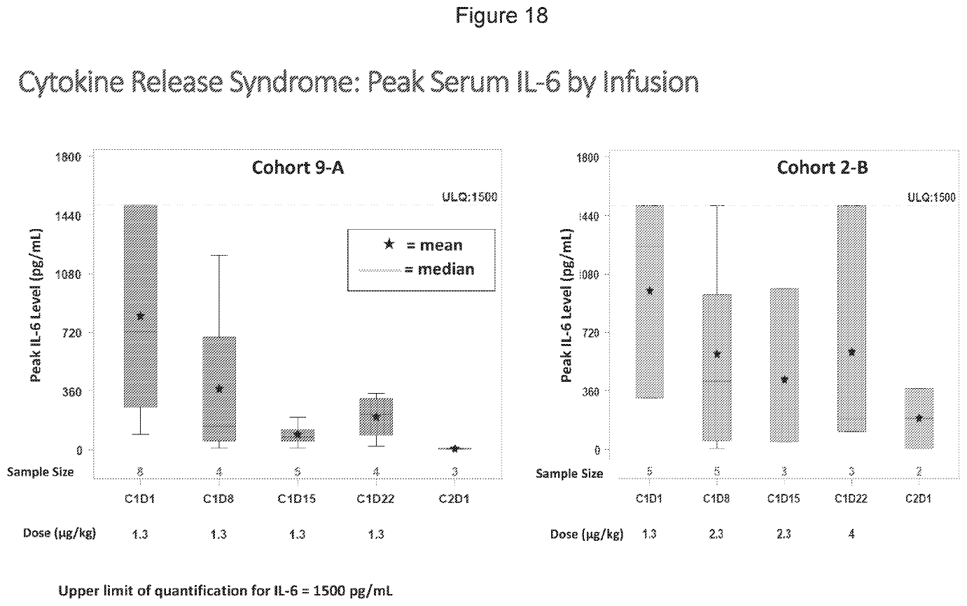

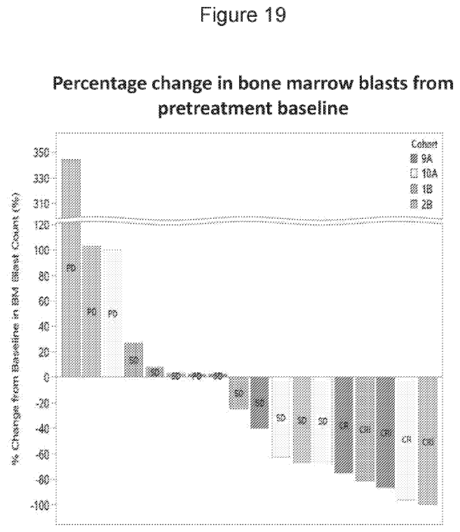

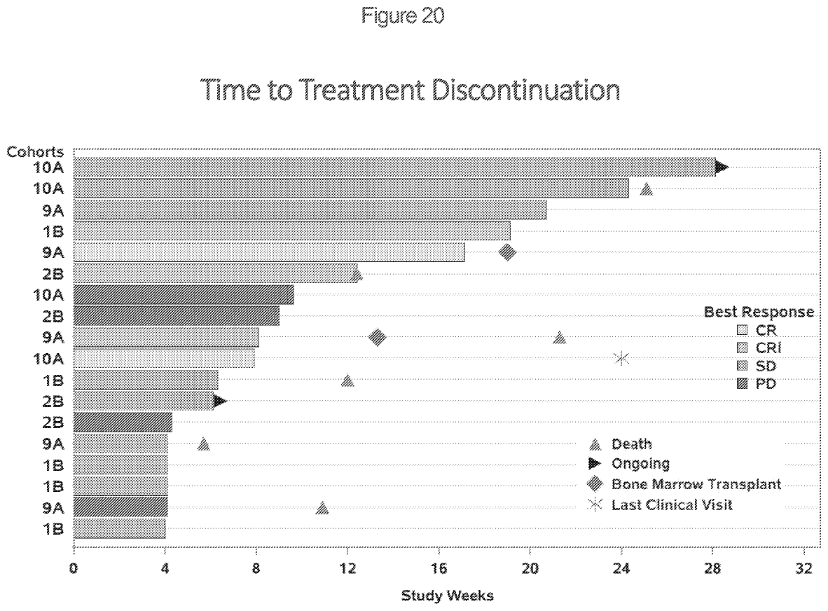

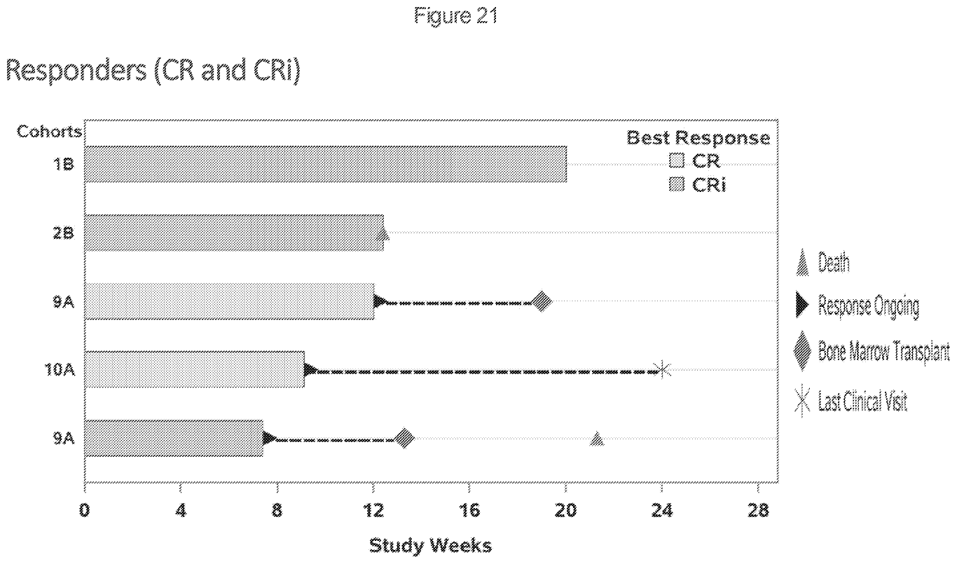

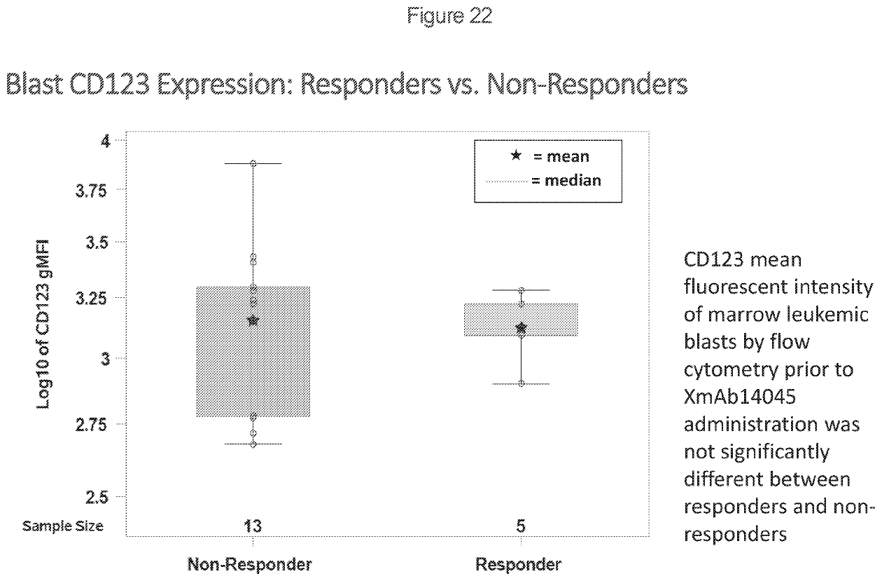

Based on published data from December 2018, from a subset of these human subjects, Peak Serum IL-6 by infusion is described in FIG. 18. Based on published data from December 2018, from a subset of these human subjects, percentage change in bone marrow blasts from pretreatment baseline is described in FIG. 19. Based on published data from December 2018, from a subset of these human subjects, the time to treatment discontinuation is described in FIG. 20. Based on published data from December 2018, from a subset of these human subjects, CR and CRi responder data is described in FIG. 21. Based on published data from December 2018, from a subset of these human subjects, blast CD123 expression, for responders versus non-responders, is described in FIG. 22.

### Example 2

### In Vitro Antitumor Efficacy

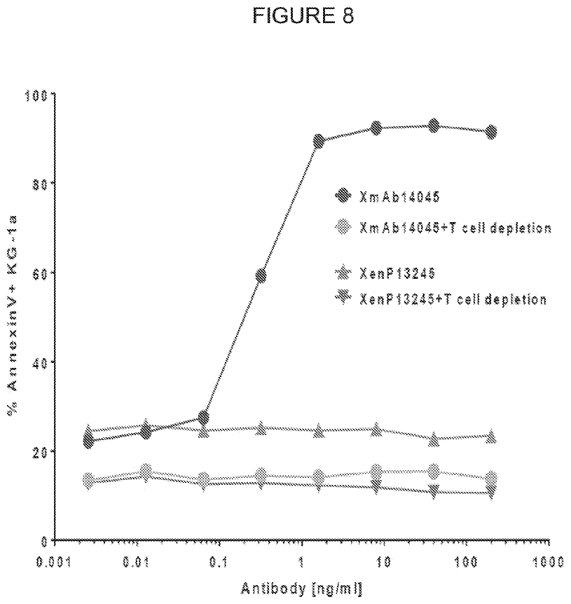

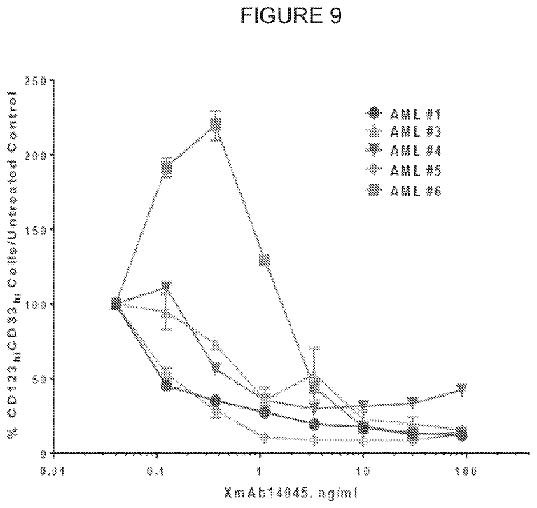

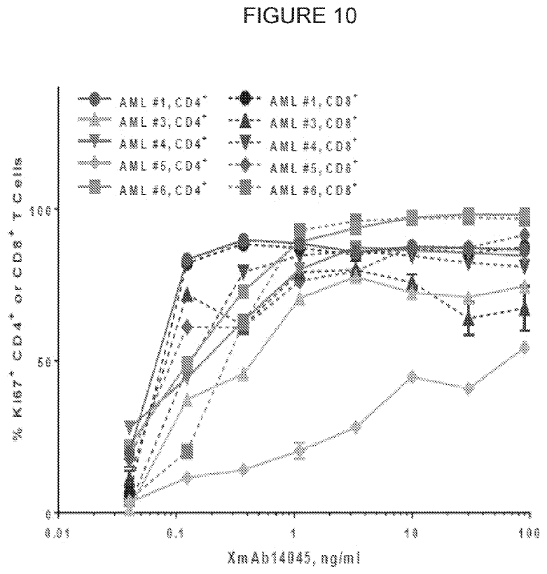

T cell-dependent cytotoxicity of XmAb14045 against CD123-positive (KG1a and Kasumi-3) and CD123-negative (Ramos) cell lines was examined using purified PBMC or T cell-depleted PBMC as effector cells. In addition, T cell activation was assessed by quantifying CD69 induction (a marker of lymphocyte activation) on both CD4+ and CD8+ T cells. XENP13245, an anti-RSV×anti-CD3 bsAb, was used as a control. XmAb14045, but not XENP13245, showed robust and potent killing of the CD123⁺ KG-1a (EC₅₀ of 0.28 ng/mL; see FIG. 8) and Kasumi-3 (EC₅₀ of 0.01 ng/mL) cell lines when supplied with human PBMC as an effector population along with robust CD69 induction in both CD4⁺ and CD8⁺ T cells. However, when T cells were depleted from PBMC (FIG. 8), XmAb14045 failed to induce killing or induce CD69 expression on T cells. XmAb14045 did not induce cytotoxicity of the CD123⁻ Ramos B cell line or induce T cell activation as measured by CD69 expression.

A series of studies was performed to evaluate the functionality of T-cells derived from AML human subject-derived PBMC. In particular, the ability of XmAb14045 to mediate RTCC towards various target populations found within, or added to, the AML samples was investigated. The target populations included: 1) a CD123ₕᵢCD33ₕᵢ population that arises in both AML PBMC and healthy PBMC upon incubation in culture for several days; 2) putative AML blast cells identified in the samples by flow cytometry; and 3) added KG1a AML cells. CD123-dependent T cell activation was measured by CD25 and Ki-67 upregulation on T cells. CD123-dependent target cell killing was monitored using annexin-V staining and by monitoring the reduction of counted blast cells.

Multiple AML human subject PBMC and normal PBMC samples were tested for XmAb14045-induced target cell killing and T cell activation. Both AML and normal PBMC contained CD123_{high} and CD33_{high} (CD123ₕᵢCD33ₕᵢ) cells; therefore, this population likely does not represent leukemic blast cells, but does serve as a useful surrogate target population. After 6 days incubation of PBMCs with XmAb14045, dose-dependent partial depletion of CD123ₕᵢCD33ₕᵢ cells was induced in AML human subject-derived PBMC, accompanied by CD4⁺ and CD8+ T cell activation and proliferation.

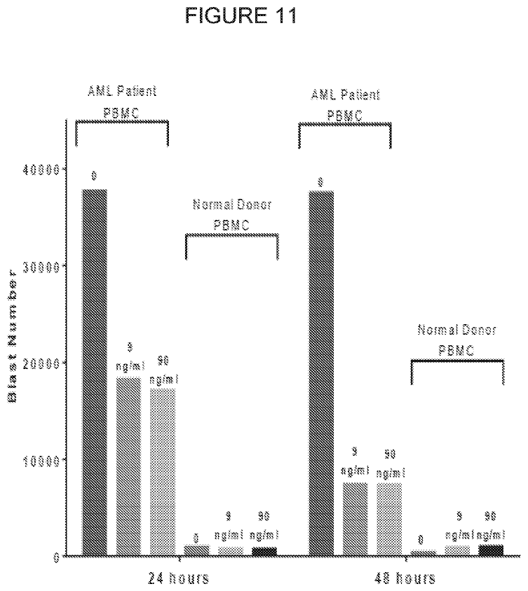

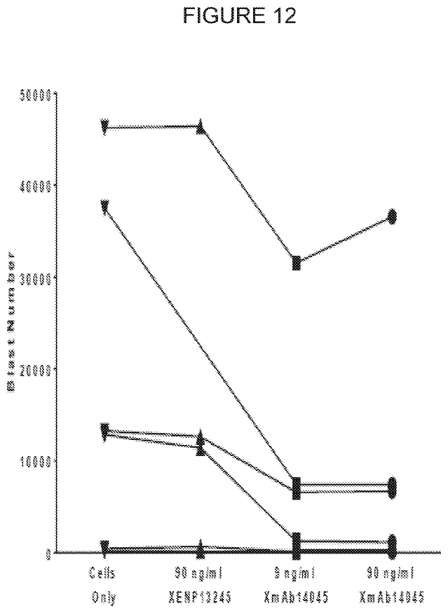

In a second set of studies, a modified staining process was used to detect leukemic blast cells in PBMC from a human subject with AML. AML PBMCs or PBMCs from a normal control donor were incubated for 24 or 48 hours with XmAb14045 at concentrations of 9 or 90 ng/mL and the putative blast cell number was obtained by flow cytometry. XmAb14045 reduced blast number by approximately 80% at 48 hours (FIG. 11). As expected, no blasts were seen in the normal donor PBMCs. This result was extended by assessing a total of 6 AML human subjects. XmAb14045 at concentrations of 9 or 90 ng/mL or XENP13245 (anti-RSV×anti-CD3) as a negative control. XmAb14045 depleted this putative blast cell population in AML PBMC at 48 hours by approximately 20% to 90%, with no apparent dependence on the number of target cells or T cells in the samples (see FIG. 12). The depletion was again associated with activation and proliferation of T cells.

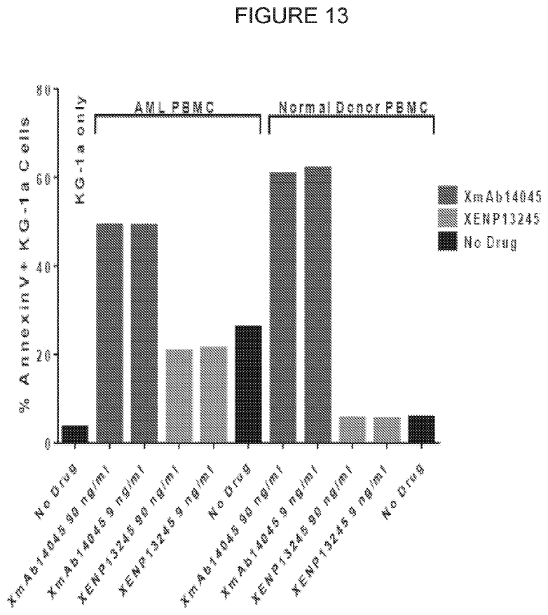

In a third set of studies, killing of an AML tumor cell line by AML human subject T cells was assessed. PBMC from one AML donor was mixed with the CD123-expressing cell line KG-1a in the presence of XmAb14045 for 48 hours (see FIG. 13). At 48 hours, XmAb14045 with AML human subject-derived PBMC induced robust apoptosis (approximately 50% annexin-V positivity), albeit still slightly lower than that induced with normal PBMC. XmAb14045 again induced robust proliferation of both AML human subject and healthy donor CD4⁺ and CD8⁺ T cells.

In summary, XmAb14045 induced allogeneic CD123⁺ KG-1a tumor cell killing by both AML human subject-derived and normal PBMC. More importantly, XmAb14045 induced autologous leukemic blast cell killing in PBMC from multiple AML human subject samples, suggesting that it could also stimulate depletion of leukemic blast cells in AML human subjects. Additionally, XmAb14045 in the presence of CD123⁺ target cells induced both CD4⁺ and CD8⁺ T cell activation in AML human subject and normal PBMC, indicating that AML human subject T cells are fully functional and capable of responding to XmAb14045.

### Example 3

### Antitumor Activity in a Mouse AML Xenograft Model

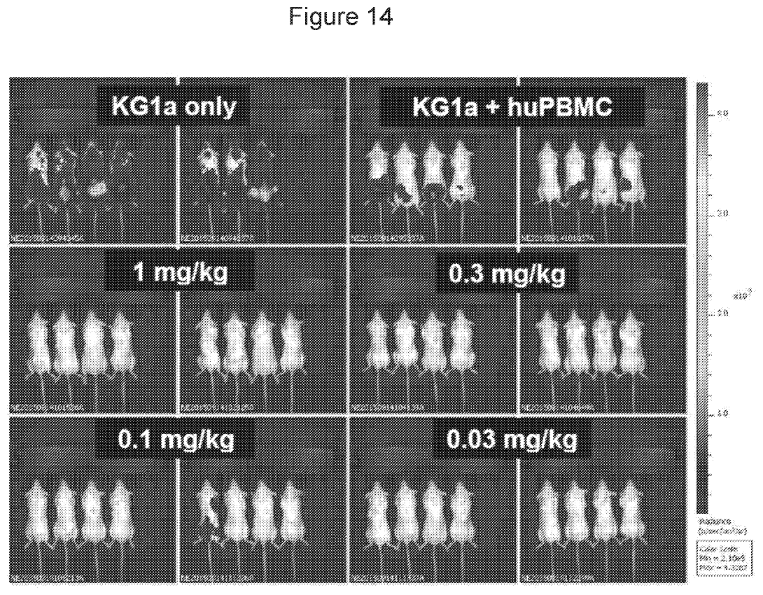

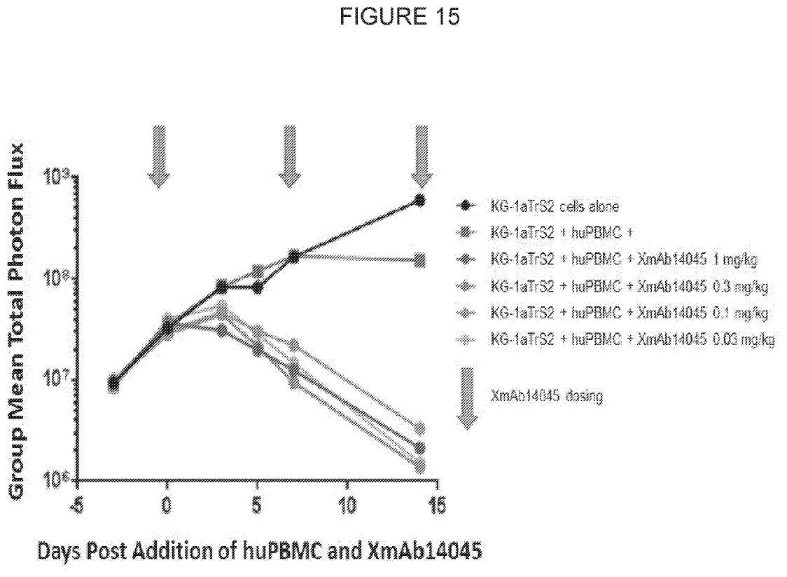

The anti-tumor activity of varying doses of XmAb14045 was examined in NSG mice that were engrafted systemically with KG1aTrS2 cells and normal human PBMCs. KG1aTrS2 cells are derived from the AML cell line KG1a, and have been engineered to express luciferase to allow quantification of tumor burden. Mice received 1×10⁶ KG1aTrS2 cells IV on Day 0. Twenty-two days after injection of KG1aTrS2 cells, mice were engrafted intraperitoneally (IP) with 10×10⁶ PBMC and were treated with 0.03, 0.1, 0.3 or 1.0 mg/kg of XmAb14045 or vehicle once a week for 3 consecutive weeks. Tumor burden was monitored throughout the study by in vivo imaging (FIG. 14). As shown in FIG. 14 and FIG. 15, mice receiving KG1a cells alone or KG1a cells plus PBMC displayed steadily increasing AML burden over time. In contrast, all tested dose levels of XmAb14045 began reducing tumor burden approximately 3 days after the initial dose, ultimately reducing burden by approximately 3 orders of magnitude relative to the KG1a-only control group, and significantly compared to the KG1a-plus-huPBMC group. No significant differences in anti-tumor activity were observed across the XmAb14045 dose range, suggesting that even lower doses would likely still exhibit anti-tumor activity.

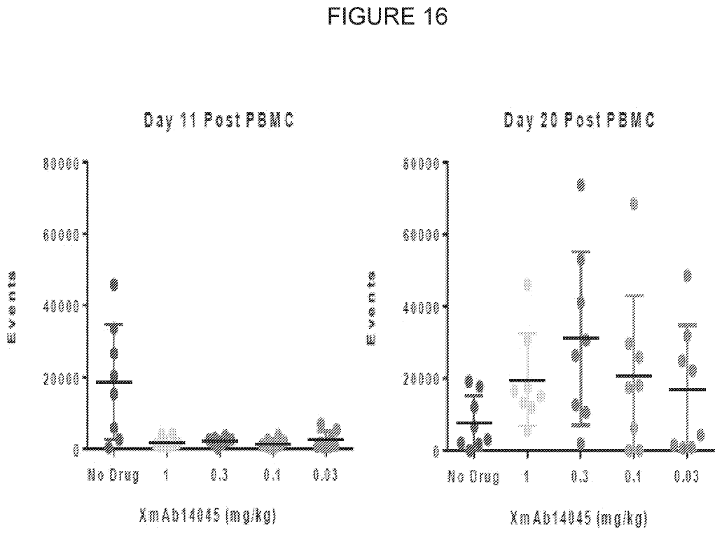

Peripheral blood samples were analyzed by flow cytometry. At Day 11, CD4⁺ and CD8⁺ T cell numbers were decreased in the treated mice compared to control, but by Day 20 this difference was no longer apparent, with a trend toward an increase in T cell counts, suggesting T cell activation and expansion mediated by XmAb14045 (FIG. 16). As another sign of T cell activation, PD1 expression was consistently higher on T cell samples from the XmAb14045-treated groups. However, it is unclear from this study whether the increase in PD1 expression interferes with the activity of XmAb14045.

What is claimed is:

1. A method for treating a CD123-expressing cancer in a human subject in need of treatment thereof, comprising administering to the human subject a bispecific anti-CD123×anti-CD3 antibody in at least a first and a second phase,
wherein during the first phase, the bispecific anti-CD123×anti-CD3 antibody is administered to the human subject in an amount of between about 700 ng/kg and about 1,900 ng/kg, once a week, for one or two weeks, and
wherein during the second phase, the bispecific anti-CD123×anti-CD3 antibody is administered to the human subject in an amount of between about 2,000 ng/kg and about 5,000 ng/kg, once a week, for at least one week.

2. The method of claim 1, wherein during the first and/or second phase, the bispecific anti-CD123×anti-CD3 antibody is administered over about two hours.

3. The method of claim 1 or 2, wherein the second phase has a duration of one or two weeks.

4. The method of claim 1 or 2, wherein the second phase is maintained until remission.

5. The method of claim 4, further comprising administering a maintenance dose.

6. The method of claim 5, wherein the maintenance dose comprises the same amount of the bispecific anti-CD123×anti-CD3 antibody administered in the second phase.

7. The method of claim 5 or 6, wherein the maintenance dose is administered once every two weeks for at least one dose.

8. The method of any one of claims 5 to 7, wherein the maintenance dose is administered once every three or four weeks or once a month for at least one dose.

9. The method of claim 4, further comprising a third phase wherein the bispecific anti-CD123×anti-CD3 antibody is administered to the human subject in an amount of between about 3,000 ng/kg and about 11,000 ng/kg, once a week for at least one week.

10. The method of claim 9, wherein during the third phase, the bispecific anti-CD123×anti-CD3 antibody is administered over about two hours.

11. The method of claim 9 or 10, wherein the third phase has a duration of one or two weeks.

12. The method of claim 9 or 10, wherein the third phase is maintained until remission.

13. The method of claim 9, further comprising administering a maintenance dose.

14. The method of claim 13, wherein the maintenance dose comprises the same amount of the bispecific anti-CD123×anti-CD3 antibody administered in the third phase.

15. The method of claim 13 or 14, wherein the maintenance dose is administered once every two weeks for at least one dose.

16. The method of any one of claims 13 to 16, wherein the maintenance dose is administered once every three or four weeks or once a month for at least one dose.

17. The method of claim 11, further comprising a fourth phase, wherein the bispecific anti-CD123×anti-CD3 antibody is administered to the human subject in an amount of between about 3,000 ng/kg and about 11,000 ng/kg, once a week for at least one week.

18. The method of claim 17, wherein during the fourth phase, the bispecific anti-CD123×anti-CD3 antibody is administered over about two hours.

19. The method of claim 17 or 18, wherein the fourth phase is maintained until remission.

20. The method of claim 13, further comprising administering a maintenance dose.

21. The method of claim 20, wherein the maintenance dose comprises the same amount of the bispecific anti-CD123×anti-CD3 antibody administered in the fourth phase.

22. The method of claim 20 or 21, wherein the maintenance dose is administered once every two weeks for at least one dose.

23. The method of any one of claims 20 to 22, wherein the maintenance dose is administered once every three or four weeks or once a month for at least one dose.

24. The method of any one of claims 1 to 23, wherein during the first phase, the bispecific anti-CD123×anti-CD3 antibody is administered to the human subject in an amount of between about 1,150 ng/kg and about 1,450 ng/kg.

25. The method of any one of claims 1 to 24, wherein during the first phase, the bispecific anti-CD123×anti-CD3 antibody is administered to the human subject in an amount of between about 700 ng/kg and about 800 ng/kg.

26. The method of any one of claims 1 to 4 and 24 to 35, consisting essentially of a first phase and a second phase,
wherein the first phase is one week, and
wherein during the second phase, the bispecific anti-CD123×anti-CD3 antibody is administered to the human subject in an amount of between about 2,200 ng/kg and about 2,400 ng/kg, once a week, until remission.

27. The method of any one of claims 1 to 3, 9 to 11 and 24 to 25, consisting essentially of a first, second, and third phase,
wherein the first phase is one week,
wherein during the second phase, the bispecific anti-CD123×anti-CD3 antibody is administered to the human subject in an amount of between about 2,200 ng/kg and about 2,400 ng/kg, once a week, for two weeks, and
wherein during the third phase, the bispecific anti-CD123×anti-CD3 antibody is administered to the human subject in an amount of between about 3,750 ng/kg and about 4,250 ng/kg, once a week, until remission.

28. The method of any one of claims 1 to 3, 9 to 11, and 17 to 19, consisting essentially of a first, second, third, and fourth phase,
wherein the first phase is one week,
wherein during the second phase, the bispecific anti-CD123×anti-CD3 antibody is administered to the human subject in an amount of between about 1,200 ng/kg and about 2,400 ng/kg, once a week, for one week,
wherein during the third phase, the bispecific anti-CD123×anti-CD3 antibody is administered to the human subject in an amount of between about 3,750 ng/kg and about 4,250 ng/kg, once a week, for one week, and
wherein during the fourth phase, the bispecific anti-CD123×anti-CD3 antibody is administered to the human subject in an amount of between about 6,500 ng/kg and about 7,500 ng/kg, once a week, until remission.

29. The method of any one of claims 1 to 3, 9 to 11, and 17 to 19, consisting essentially of a first, second, third, and fourth phase,
wherein the first phase is one week,
wherein during the second phase, the bispecific anti-CD123×anti-CD3 antibody is administered to the human subject in an amount of between about 3,750 ng/kg and about 4,250 ng/kg, once a week, for one week,
wherein during the third phase, the bispecific anti-CD123×anti-CD3 antibody is administered to the human subject in an amount of between about 6,500 ng/kg and about 7,500 ng/kg, once a week, for one week, and
wherein during the fourth phase, the bispecific anti-CD123×anti-CD3 antibody is administered to the human subject in an amount of between about 11,000 ng/kg and about 13,000 ng/kg, once a week, until remission.

30. The method of any one of claims 1 to 29, wherein the bispecific anti-CD123×anti-CD3 antibody is administered intravenously.

31. The method of any one of claims 28 to 30, wherein during the third and/or fourth phases, the bispecific anti-CD123×anti-CD3 antibody is administered over about two hours.

32. A method for treating a CD123-expressing cancer in a human subject in need of treatment thereof, comprising administering to the human subject a bispecific anti-CD123×anti-CD3 antibody in at least a first phase and a second phase and a third phase,
wherein during the first phase, the bispecific anti-CD123×anti-CD3 antibody is administered to the human subject in an amount of between about 300 ng/kg and about 1,100 ng/kg, three times a week, for one week, with the proviso that the first dose amount of the first phase is not greater than about 770 ng/kg,
wherein during the second phase, the bispecific anti-CD123×anti-CD3 antibody is administered to the human subject in an amount of between about 300 ng/kg and about 1,100 ng/kg, three times a week, for one week, and
wherein during the third phase, the bispecific anti-CD123×anti-CD3 antibody is administered to the human subject in an amount of between about 900 ng/kg and about 3,400 ng/kg, once a week for at least one week.

33. The method of claim 32, wherein during the first phase, the bispecific anti-CD123×anti-CD3 antibody is administered to the human subject in an amount of between about 400 ng/kg and about 450 ng/kg, three times a week, for one week, and
wherein during the second phase, the bispecific anti-CD123×anti-CD3 antibody is administered to the human subject in an amount of between about 400 ng/kg and about 450 ng/kg, three times a week, for one week
wherein during the third phase, the bispecific anti-CD123×anti-CD3 antibody is administered to the human subject in an amount of between about 1,150 ng/kg and about 1,450 ng/kg, once a week for at least one week.

34. The method of claim 32 or 33, wherein during the first phase, the bispecific anti-CD123×anti-CD3 antibody is administered to the human subject, three times a week, for one week, where the first dose amount in the first phase is about 750 ng/kg, and the subsequent two dose amounts in the first phase are between about 760 ng/kg and about 780 ng/kg and
wherein during the second phase, the bispecific anti-CD123×anti-CD3 antibody is administered to the human subject in an amount of between about 760 ng/kg and about 780 ng/kg, three times a week, for one week, and
wherein during the third phase, the bispecific anti-CD123×anti-CD3 antibody is administered to the human subject in an amount of between about 2,200 ng/kg and about 2,400 ng/kg, once a week for at least one week.

35. The method of any one of claims 32 to 34, wherein during the first phase, the bispecific anti-CD123×anti-CD3 antibody is administered to the human subject, three times a week, for one week, where the first dose amount in the first phase is about 750 ng/kg, and the subsequent two dose amounts in the first phase are between about 1,150 ng/kg and about 1,450 ng/kg
wherein during the second phase, the bispecific anti-CD123×anti-CD3 antibody is administered to the human subject in an amount of between about 1,150 ng/kg and about 1,450 ng/kg, three times a week, for one week, and
wherein during the third phase, the bispecific anti-CD123×anti-CD3 antibody is administered to the human subject in an amount of between about 3,750 ng/kg and 4,250 ng/kg, once a week for at least one week.

36. The method of any one of claims 32 to 35, wherein during the first and/or second and/or third phase, the bispecific anti-CD123×anti-CD3 antibody is administered over about two hours.

37. The method of any one of claims 32 to 36, wherein the bispecific anti-CD123×anti-CD3 antibody is administered intravenously.

38. The method of any one of claims 32 to 37, wherein the second phase is maintained until remission.

39. The method of claim 38, further comprising administering a maintenance dose.

40. The method of claim 39, wherein the maintenance dose comprises the same amount of the bispecific anti-CD123×anti-CD3 antibody administered in the second phase.

41. The method of claim 39 or 40, wherein the maintenance dose is administered once every two weeks for at least one dose.

42. The method of any one of claims 39 to 41, wherein the maintenance dose is administered once every three or four weeks or once a month for at least one dose.

43. A method for treating a CD123-expressing cancer in a human subject in need of treatment thereof, comprising administering to the human subject a bispecific anti-CD123×anti-CD3 antibody in an amount of between about 900 ng/kg and about 3,400 ng/kg, once a week for at least one week.

44. The method of claim 43, wherein the bispecific anti-CD123×anti-CD3 antibody is administered in an amount of between about 1,150 ng/kg and 1,450 ng/kg.

45. The method of claim 43 or 44, wherein the bispecific anti-CD123×anti-CD3 antibody is administered in an amount of between about 2,200 ng/kg and 2,400 ng/kg.

46. The method of any one of claims 1 to 45, wherein the CD123-expressing cancer is a hematologic cancer.

47. The method of any one of claims 1 to 46, wherein the CD123-expressing cancer is a leukemia.

48. The method of any one of claims 1 to 47, wherein the CD123-expressing cancer is selected from the group consisting of acute myeloid leukemia (AML), chronic myeloid leukemia (CML), acute lymphocytic leukemia (ALL), and hairy cell leukemia (HCL).

49. The method of any one of claims 1 to 48, wherein the CD123-expressing cancer is acute myeloid leukemia (AML).

50. The method of claim 49, wherein the acute myeloid leukemia (AML) is blastic plasmacytoid dendritic cell neoplasm (BPDCN).

51. The method of any one of claims 1 to 50, wherein the CD123-expressing cancer is acute lymphocytic leukemia, and the acute lymphocytic leukemia is B-cell acute lymphocytic leukemia (B-ALL).

52. The method of any one of claims 1 to 51, wherein the remission is a reduction in the number of CD123-expressing cancer cells or reduction in the rate of growth of CD123-expressing cancer cells.

53. The method of any one of claims 1 to 52, wherein the remission is an increase in T cell activation or an increase in IFN pathway upregulation.

54. The method of any one of claims 1 to 53, wherein the remission is a partial remission of the CD123-expressing cancer.

55. The method of any one of claims 1 to 54, wherein the bispecific anti-CD123×anti-CD3 antibody comprises a Heavy Chain 1 (HC1) (Fab-Fc) set forth in SEQ ID NO:1, a Heavy Chain 2 (HC2) (scFv-Fc) set forth in SEQ ID NO: 2 and a Light Chain set forth in SEQ ID NO: 3.

56. The method of claim 55, wherein the bispecific anti-CD123×anti-CD3 antibody consists of a Heavy Chain 1 (HC1) (Fab-Fc) set forth in SEQ ID NO:1, a Heavy Chain 2 (HC2) (scFv-Fc) set forth in SEQ ID NO: 2 and a Light Chain set forth in SEQ ID NO: 3.

57. The method of any one of claims 1 to 56, further comprising assessing the weight of the human subject prior to the administering of the first phase of the bispecific anti-CD123×anti-CD3 antibody.

58. The method of any one of claims 1 to 57, further comprising administering to the human subject one or more therapeutic agents prior to the administering of the first phase of the bispecific anti-CD123×anti-CD3 antibody.

59. The method of claim 58, wherein the one or more therapeutic agents ameliorates the side effects of the bispecific anti-CD123×anti-CD3 antibody administration.

60. The method of claim 59, wherein the one or more therapeutic agents is a steroid, an antihistamine, an anti-allergic agent, an antinausea agent (or anti-emetic), an analgesic agent, an antipyretic agent, a cytoprotective agent, a vasopressor agent, an anticonvulsant agent, an anti-inflammatory agent, or any combination thereof.

61. The method of any one of claims 58 to 60, wherein the one or more therapeutic agents is a combination of a corticosteroid, diphenhydramine, and acetaminophen.


SEQUENCE LISTING (18 sequences)

SEQ ID NO: 1 - protein, 449 aa, Artificial Sequence
  XENP14045 Anti-CD123 x Anti-CD3 Fab-scFv-Fc Heavy Chain 1
QVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 2 - protein, 485 aa, Artificial Sequence
  XENP14045 Anti-CD123 x Anti-CD3 Fab-scFv-Fc Heavy Chain 2
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 220 aa, Artificial Sequence
  XENP14045 Anti-CD123 x Anti-CD3 Fab-scFv-Fc Light Chain
DFVMTQSPDSLAVSLGERATINCKSSQSLLNTGNQKNYLTWYQQKPGQPPKLLIYWASTRESGVPDRFTGSGSGTDFTLTISSLQAEDVAVYYCQNDYSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 225 aa, Artificial Sequence
  XENP13760 7G3_H0L0_Fab_His Heavy chain
EVQLQQSGPELVKPGASVKMSCKASGYTFTDYYMKWVKQSHGKSLEWIGDIIPSNGATFYNQKFKGKATLTVDRSSSTAYMHLNSLTSEDSAVYYCTRSHLLRASWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGS

SEQ ID NO: 5 - protein, 220 aa, Artificial Sequence
  XENP13760 7G3_H0L0_Fab_His Light chain
DFVMTQSPSSLTVTAGEKVTMSCKSSQSLLNSGNQKNYLTWYLQKPGQPPKLLIYWASTRESGVPDRFTGSGSGTDFTLTISSVQAEDLAVYYCQNDYSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 225 aa, Artificial Sequence
  XENP13761 7G3_H1L1_Fab_His Heavy chain
QVQLVQSGAEVKKPGASVKVSCKASGYTFTDYYMKWVRQAPGQSLEWMGDIIPSNGATFYNQKFQGRVTMTVDRSTSTAYMELSSLRSEDTAVYYCTRSHLLRASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGS

SEQ ID NO: 7 - protein, 220 aa, Artificial Sequence
  XENP13761 7G3_H1L1_Fab_His Light chain
DFVMTQSPDSLAVSLGERATINCKSSQSLLNSGNQKNYLTWYQQKPGQPPKLLIYWASTRESGVPDRFTGSGSGTDFTLTISSLQAEDVAVYYCQNDYSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 225 aa, Artificial Sequence
  XENP13961 7G3_H1.107_L1_Fab_His Heavy chain
QVQLQQSGAEVKKPGASVKVSCKASGYTFTDYYMKWVRQAPGQSLEWMGDIIPSNGATFYNQKFKGKATLTVDRSTSTAYMELSSLRSEDTAVYYCARSHLLRASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGS

SEQ ID NO: 9 - protein, 220 aa, Artificial Sequence; identical to SEQ ID NO: 7
  XENP13961 7G3_H1.107_L1_Fab_His Light chain

SEQ ID NO: 10 - protein, 225 aa, Artificial Sequence
  XENP13963 7G3_H1.109_L1_Fab_His Heavy chain
QVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGS

SEQ ID NO: 11 - protein, 220 aa, Artificial Sequence; identical to SEQ ID NO: 7
  XENP13963 7G3_ H1.109_L1_Fab_His Light chain

SEQ ID NO: 12 - protein, 225 aa, Artificial Sequence; identical to SEQ ID NO: 8
  XENP13965 7G3_H1.107_L1.57_Fab_His Heavy chain

SEQ ID NO: 13 - protein, 220 aa, Artificial Sequence; identical to SEQ ID NO: 3
  XENP13965 7G3_H1.107_L1.57_Fab_His Light chain

SEQ ID NO: 14 - protein, 225 aa, Artificial Sequence; identical to SEQ ID NO: 10
  XENP13967 7G3_H1.109_L1.57_Fab_His Heavy chain

SEQ ID NO: 15 - protein, 220 aa, Artificial Sequence; identical to SEQ ID NO: 3
  XENP13967 7G3_H1.109_L1.57_Fab_His Light chain

SEQ ID NO: 16 - protein, 449 aa, Artificial Sequence
  XENP13928 Anti-CD123 x Anti-CD3 Fab-scFv-Fc Heavy Chain 1
EVQLQQSGPELVKPGASVKMSCKASGYTFTDYYMKWVKQSHGKSLEWIGDIIPSNGATFYNQKFKGKATLTVDRSSSTAYMHLNSLTSEDSAVYYCTRSHLLRASWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 17 - protein, 485 aa, Artificial Sequence; identical to SEQ ID NO: 2
  XENP13928 Anti-CD123 x Anti-CD3 Fab-scFv-Fc Heavy Chain 2

SEQ ID NO: 18 - protein, 220 aa, Artificial Sequence; identical to SEQ ID NO: 5
  XENP13928 Anti-CD123 x Anti-CD3 Fab-scFv-Fc Light Chain